SEQUENCE LISTING (122 sequences)

SEQ ID NO: 1 - DNA, 72 bp, Artificial Sequence
  Human CD8 Transmembrane domain
atctacatctgggcgcccttggccgggacttgtggggtccttctcctgtcactggttatcaccctttactgc

SEQ ID NO: 2 - DNA, 135 bp, Artificial Sequence
  Human CD8 hinge/spacer domain
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 3 - DNA, 4832 bp, Artificial Sequence
  pBA9b plasmid sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgctcgcgagtcgacaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 336 bp, Artificial Sequence
  Human CD3zeta intracellular domain
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 5 - DNA, 126 bp, Artificial Sequence
  Human 4-1BB intracellular domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 6 - DNA, 63 bp, Artificial Sequence
  Leader sequence
atggccctgcctgtgacagccctgctgctgcctctggctctgctgctgcatgccgctagaccc

SEQ ID NO: 7 - protein, 107 aa, Artificial Sequence
  Murine CD19 variable heavy chain
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELK

SEQ ID NO: 8 - protein, 15 aa, Artificial Sequence
  GS linker
GGGGSGGGGSGGGGS

SEQ ID NO: 9 - protein, 116 aa, Artificial Sequence
  Murine CD19 variable light chain
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

SEQ ID NO: 10 - DNA, 138 bp, Artificial Sequence
  Murine CD8 hinge/spacer domain
tctactactaccaagccagtgctgcgaactccctcacctgtgcaccctaccgggacatctcagccccagagaccagaagattgtcggccccgtggctcagtgaaggggaccggattggacttcgcctgtgatatttac

SEQ ID NO: 11 - DNA, 108 bp, Artificial Sequence
  Murine CD28 hinge/spacer domain
attgagttcatgtaccctccgccttacctagacaacgagaggagcaatggaactattattcacataaaagagaaacatctttgtcatactcagtcatctcctaagctg

SEQ ID NO: 12 - RNA, 1662 nt, Artificial Sequence
  pBA-9b vRNA genome
gcgccaguccuccgauugacugagucgcccggguacccguguauccaauaaacccucuugcaguugcauccgacuuguggucucgcuguuccuugggagggucuccucugagugauugacuacccgucagcgggggucuuucauuugggggcucguccgggaucgggagaccccugcccagggaccaccgacccaccaccgggagguaagcuggccagcaacuuaucugugucuguccgauugucuagugucuaugacugauuuuaugcgccugcgucgguacuaguuagcuaacuagcucuguaucuggcggacccgugguggaacugacgaguucggaacacccggccgcaacccugggagacgucccagggacuucgggggccguuuuuguggcccgaccugaguccaaaaaucccgaucguuuuggacucuuuggugcaccccccuuagaggagggauaugugguucugguaggagacgagaaccuaaaacaguucccgccuccgucugaauuuuugcuuucgguuugggaccgaagccgcgccgcgcgucuugucugcugcagcaucguucuguguugucucugucugacuguguuucuguauuugucugagaauuaaggccagacuguuaccacucccugaaguuugaccuuaggucacuggaaagaugucgagcggaucgcucacaaccagucgguagaugucaagaagagacguuggguuaccuucugcucugcagaauggccaaccuuuaacgucggauggccgcgagacggcaccuuuaaccgagaccucaucacccagguuaagaucaaggucuuuucaccuggcccgcauggacacccagaccagguccccuacaucgugaccugggaagccuuggcuuuugaccccccucccugggucaagcccuuuguacacccuaagccuccgccuccucuuccuccauccgccccgucucucccccuugaaccuccucguucgaccccgccucgauccucccuuuauccagcccucacuccuucucuaggcgccggaauuaauucucgaggggcccagaucugcggccgcucgcgagucgacaagcuuggauccaucgauaaaauaaaagauuuuauuuagucuccagaaaaaggggggaaugaaagaccccaccuguagguuuggcaagcuagcuuaaguaacgccauuuugcaaggcauggaaaaauacauaacugagaauagagaaguucagaucaaggucaggaacagauggaacagcugaauaugggccaaacaggauaucugugguaagcaguuccugccccggcucagggccaagaacagauggaacagcugaauaugggccaaacaggauaucugugguaagcaguuccugccccggcucagggccaagaacagaugguccccagaugcgguccagcccucagcaguuucuagagaaccaucagauguuuccagggugccccaaggaccugaaaugacccugugccuuauuugaacuaaccaaucaguucgcuucucgcuucuguucgcgcgcuucugcuccccgagcucaauaaaagagcccacaaccccucacucggcgcgccaguccuccgauugacugagucgcccggguacccguguauccaauaaacccucuugcaguugca

SEQ ID NO: 13 - DNA, 1475 bp, Artificial Sequence
  hCD19CAR1
gcggccgcatggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaggtcgac

SEQ ID NO: 14 - DNA, 1475 bp, Artificial Sequence
  hCD19CAR2
gcggccgcatggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaggtcgac

SEQ ID NO: 15 - DNA, 1490 bp, Artificial Sequence
  hCD19CAR3
gcggccgcatggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggtgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaggtcgac

SEQ ID NO: 16 - DNA, 1490 bp, Artificial Sequence
  hCD19CAR4
gcggccgcatggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaggtcgac

SEQ ID NO: 17 - DNA, 1475 bp, Artificial Sequence
  hCD19CAR5
gcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgac

SEQ ID NO: 18 - DNA, 6287 bp, Artificial Sequence
  pBA9b-hCD19CAR1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaggtcgacaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 19 - DNA, 6287 bp, Artificial Sequence
  pBA9b-hCD19CAR2
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaggtcgacaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 20 - DNA, 6302 bp, Artificial Sequence
  pBA9b-hCD19CAR3
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggtgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaggtcgacaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - DNA, 6302 bp, Artificial Sequence
  pBA9b-hCD19CAR4
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaggtcgacaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 22 - DNA, 6287 bp, Artificial Sequence
  pBA9b-hCD19CAR5
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgactctagaggatcgatccccggccgctctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaattaaggccagactgttaccactccctgaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaattctcgaggggcccagatctgcggccgcatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggtcgacaagcttggatccatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgaagccgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 23 - DNA, 288 bp, Artificial Sequence
  Adnectins (10Fn3)
  CDS (1)..(288)
gtgagcgacgtgcccagaaagctggaggtggtggccgccacccccaccagcctgctgatcagctgggacgcccccgccgtgaccgtgagatactacagaatcacctacggcgagaccggcggcaacagccccgtgcaggagttcaccgtgcccggcagcaagagcaccgccaccatcagcggcctgaagcccggcgtggactacaccatcaccgtgtacgccgtgaccggcagaggcgacagccccgccagcagcaagcccatcagcaactacagaaccgccctggag

SEQ ID NO: 24 - protein, 96 aa, Artificial Sequence
  Synthetic Construct
VSDVPRKLEVVAATPTSLLISWDAPAVTVRYYRITYGETGGNSPVQEFTVPGSKSTATISGLKPGVDYTITVYAVTGRGDSPASSKPISNYRTALE

SEQ ID NO: 25 - DNA, 303 bp, Artificial Sequence
  Adnectin 1
  CDS (1)..(303)
gtgagcgacgtgcccagaaagctggaggtggtggccgccacccccaccagcctgctgatcagctgggacagcggcagaggcagctacagatactacagaatcacctacggcgagaccggcggcaacagccccgtgcaggagttcaccgtgcccggccccgtgcacaccgccaccatcagcggcctgaagcccggcgtggactacaccatcaccgtgtacgccgtgaccgaccacaagccccacgccgacggcccccacacctaccacgagagccccatcagcaactacagaaccgccctggag

SEQ ID NO: 26 - protein, 101 aa, Artificial Sequence
  Synthetic Construct
VSDVPRKLEVVAATPTSLLISWDSGRGSYRYYRITYGETGGNSPVQEFTVPGPVHTATISGLKPGVDYTITVYAVTDHKPHADGPHTYHESPISNYRTALE

SEQ ID NO: 27 - DNA, 288 bp, Artificial Sequence
  Adnectin 2
  CDS (1)..(288)
gtgagcgacgtgcccagaaagctggaggtggtggccgccacccccaccagcctgctgatcagctgggagcacgactacccctacagaagatactacagaatcacctacggcgagaccggcggcaacagccccgtgcaggagttcaccgtgcccaaggacgtggacaccgccaccatcagcggcctgaagcccggcgtggactacaccatcaccgtgtacgccgtgaccagcagctacaagtacgacatgcagtacagccccatcagcaactacagaaccgccctggag

SEQ ID NO: 28 - protein, 96 aa, Artificial Sequence
  Synthetic Construct
VSDVPRKLEVVAATPTSLLISWEHDYPYRRYYRITYGETGGNSPVQEFTVPKDVDTATISGLKPGVDYTITVYAVTSSYKYDMQYSPISNYRTALE

SEQ ID NO: 29 - DNA, 276 bp, Artificial Sequence
  Pronectins (1Fn3)
  CDS (1)..(276)
agcggccccgtggaggtgttcatcaccgagacccccagccagcccaacagccaccccatccagtggaacgccccccagcccagccacatcagcaagtacatcctgagatggagacccaagaacagcgtgggcagatggaaggaggccaccatccccggccacctgaacagctacaccatcaagggcctgaagcccggcgtggtgtacgagggccagctgatcagcatccagcagtacggccaccaggaggtgaccagattcgacttcaccaccacc

SEQ ID NO: 30 - protein, 92 aa, Artificial Sequence
  Synthetic Construct
SGPVEVFITETPSQPNSHPIQWNAPQPSHISKYILRWRPKNSVGRWKEATIPGHLNSYTIKGLKPGVVYEGQLISIQQYGHQEVTRFDFTTT

SEQ ID NO: 31 - DNA, 270 bp, Artificial Sequence
  Pronectins 2Fn3)
  CDS (1)..(270)
agccccctggtggccaccagcgagagcgtgaccgagatcaccgccagcagcttcgtggtgagctgggtgagcgccagcgacaccgtgagcggcttcagagtggagtacgagctgagcgaggagggcgacgagccccagtacctggacctgcccagcaccgccaccagcgtgaacatccccgacctgctgcccggcagaaagtacatcgtgaacgtgtaccagagcgaggacggcgagcagagcctgatcctgagcaccagccagaccacc

SEQ ID NO: 32 - protein, 90 aa, Artificial Sequence
  Synthetic Construct
SPLVATSESVTEITASSFVVSWVSASDTVSGFRVEYELSEEGDEPQYLDLPSTATSVNIPDLLPGRKYIVNVYQSEDGEQSLILSTSQTT

SEQ ID NO: 33 - DNA, 282 bp, Artificial Sequence
  Pronectins (3Fn3)
  CDS (1)..(282)
gcccccgacgccccccccgaccccaccgtggaccaggtggacgacaccagcatcgtggtgagatggagcagaccccaggcccccatcaccggctacagaatcgtgtacagccccagcgtggagggcagcagcaccgagctgaacctgcccgagaccgccaacagcgtgaccctgagcgacctgcagcccggcgtgcagtacaacatcaccatctacgccgtggaggagaaccaggagagcacccccgtggtgatccagcaggagaccaccggcacccccaga

SEQ ID NO: 34 - protein, 94 aa, Artificial Sequence
  Synthetic Construct
APDAPPDPTVDQVDDTSIVVRWSRPQAPITGYRIVYSPSVEGSSTELNLPETANSVTLSDLQPGVQYNITIYAVEENQESTPVVIQQETTGTPR

SEQ ID NO: 35 - DNA, 270 bp, Artificial Sequence
  Pronectins (4Fn3)
  CDS (1)..(270)
accgtgcccagccccagagacctgcagttcgtggaggtgaccgacgtgaaggtgaccatcatgtggaccccccccgagagcgccgtgaccggctacagagtggacgtgatccccgtgaacctgcccggcgagcacggccagagactgcccatcagcagaaacaccttcgccgaggtgaccggcctgagccccggcgtgacctactacttcaaggtgttcgccgtgagccacggcagagagagcaagcccctgaccgcccagcagaccacc

SEQ ID NO: 36 - protein, 90 aa, Artificial Sequence
  Synthetic Construct
TVPSPRDLQFVEVTDVKVTIMWTPPESAVTGYRVDVIPVNLPGEHGQRLPISRNTFAEVTGLSPGVTYYFKVFAVSHGRESKPLTAQQTT

SEQ ID NO: 37 - DNA, 270 bp, Artificial Sequence
  Pronectins (5Fn3)
  CDS (1)..(270)
aagctggacgcccccaccaacctgcagttcgtgaacgagaccgacagcaccgtgctggtgagatggaccccccccagagcccagatcaccggctacagactgaccgtgggcctgaccagaagaggccagcccagacagtacaacgtgggccccagcgtgagcaagtaccccctgagaaacctgcagcccgccagcgagtacaccgtgagcctggtggccatcaagggcaaccaggagagccccaaggccaccggcgtgttcaccaccctg

SEQ ID NO: 38 - protein, 90 aa, Artificial Sequence
  Synthetic Construct
KLDAPTNLQFVNETDSTVLVRWTPPRAQITGYRLTVGLTRRGQPRQYNVGPSVSKYPLRNLQPASEYTVSLVAIKGNQESPKATGVFTTL

SEQ ID NO: 39 - DNA, 258 bp, Artificial Sequence
  Pronectins (6Fn3)
  CDS (1)..(258)
cagcccggcagcagcatccccccctacaacaccgaggtgaccgagaccaccatcgtgatcacctggacccccgcccccagactgggcttcaagctgggcgtgagacccagccagggcggcgaggcccccagagaggtgaccagcgacagcggcagcgtggtgagcggcctgacccccggcgtggagtacgtgtacaccatccaggtgctgagagacggccaggagagagacgcccccatcgtgaacaaggtggtgacc

SEQ ID NO: 40 - protein, 86 aa, Artificial Sequence
  Synthetic Construct
QPGSSIPPYNTEVTETTIVITWTPAPRLGFKLGVRPSQGGEAPREVTSDSGSVVSGLTPGVEYVYTIQVLRDGQERDAPIVNKVVT

SEQ ID NO: 41 - DNA, 282 bp, Artificial Sequence
  Pronectins (7Fn3)
  CDS (1)..(282)
cccctgagcccccccaccaacctgcacctggaggccaaccccgacaccggcgtgctgaccgtgagctgggagagaagcaccacccccgacatcaccggctacagaatcaccaccacccccaccaacggccagcagggcaacagcctggaggaggtggtgcacgccgaccagagcagctgcaccttcgacaacctgagccccggcctggagtacaacgtgagcgtgtacaccgtgaaggacgacaaggagagcgtgcccatcagcgacaccatcatcccctga

SEQ ID NO: 42 - protein, 93 aa, Artificial Sequence
  Synthetic Construct
PLSPPTNLHLEANPDTGVLTVSWERSTTPDITGYRITTTPTNGQQGNSLEEVVHADQSSCTFDNLSPGLEYNVSVYTVKDDKESVPISDTIIP

SEQ ID NO: 43 - DNA, 276 bp, Artificial Sequence
  Pronectins (8Fn3)
  CDS (1)..(276)
gccgtgcccccccccaccgacctgagattcaccaacatcggccccgacaccatgagagtgacctgggcccccccccccagcatcgacctgaccaacttcctggtgagatacagccccgtgaagaacgaggaggacgtggccgagctgagcatcagccccagcgacaacgccgtggtgctgaccaacctgctgcccggcaccgagtacgtggtgagcgtgagcagcgtgtacgagcagcacgagagcacccccctgagaggcagacagaagacctga

SEQ ID NO: 44 - protein, 91 aa, Artificial Sequence
  Synthetic Construct
AVPPPTDLRFTNIGPDTMRVTWAPPPSIDLTNFLVRYSPVKNEEDVAELSISPSDNAVVLTNLLPGTEYVVSVSSVYEQHESTPLRGRQKT

SEQ ID NO: 45 - DNA, 273 bp, Artificial Sequence
  Pronectins (9Fn3)
  CDS (1)..(273)
ggcctggacagccccaccggcatcgacttcagcgacatcaccgccaacagcttcaccgtgcactggatcgcccccagagccaccatcaccggctacagaatcagacaccaccccgagcacttcagcggcagacccagagaggacagagtgccccacagcagaaacagcatcaccctgaccaacctgacccccggcaccgagtacgtggtgagcatcgtggccctgaacggcagagaggagagccccctgctgatcggccagcagagcacctga

SEQ ID NO: 46 - protein, 90 aa, Artificial Sequence
  Synthetic Construct
GLDSPTGIDFSDITANSFTVHWIAPRATITGYRIRHHPEHFSGRPREDRVPHSRNSITLTNLTPGTEYVVSIVALNGREESPLLIGQQST

SEQ ID NO: 47 - DNA, 279 bp, Artificial Sequence
  Pronectins (10Fn3)
  CDS (1)..(279)
gtgagcgacgtgcccagagacctggtggtggccgccacccccaccagcctgctgatcagctgggacgcccccgccgtgaccgtgagatactacagaatcacctacggcgagaccggcggcaacagccccgtgcaggagttcaccgtgcccggcagcaagagcaccgccaccatcagcggcctgaagcccggcgtggactacaccatcaccgtgtacgccgtgaccggcagaggcgacagccccgccagcagcaagcccatcagcatcaactacagaacc

SEQ ID NO: 48 - protein, 93 aa, Artificial Sequence
  Synthetic Construct
VSDVPRDLVVAATPTSLLISWDAPAVTVRYYRITYGETGGNSPVQEFTVPGSKSTATISGLKPGVDYTITVYAVTGRGDSPASSKPISINYRT

SEQ ID NO: 49 - DNA, 270 bp, Artificial Sequence
  Pronectins (11Fn3)
  CDS (1)..(270)
gagatcgacaagcccagccagatgcaggtgaccgacgtgcaggacaacagcatcagcgtgaagtggctgcccagcagcagccccgtgaccggctacagagtgaccaccacccccaagaacggccccggccccaccaagaccaagaccgccggccccgaccagaccgagatgaccatcgagggcctgcagcccaccgtggagtacgtggtgagcgtgtacgcccagaaccccagcggcgagagccagcccctggtgcagaccgccgtgacc

SEQ ID NO: 50 - protein, 90 aa, Artificial Sequence
  Synthetic Construct
EIDKPSQMQVTDVQDNSISVKWLPSSSPVTGYRVTTTPKNGPGPTKTKTAGPDQTEMTIEGLQPTVEYVVSVYAQNPSGESQPLVQTAVT

SEQ ID NO: 51 - DNA, 273 bp, Artificial Sequence
  Pronectins (12Fn3)
  CDS (1)..(273)
aacatcgacagacccaagggcctggccttcaccgacgtggacgtggacagcatcaagatcgcctgggagagcccccagggccaggtgagcagatacagagtgacctacagcagccccgaggacggcatccacgagctgttccccgcccccgacggcgaggaggacaccgccgagctgcagggcctgagacccggcagcgagtacaccgtgagcgtggtggccctgcacgacgacatggagagccagcccctgatcggcacccagagcacctga

SEQ ID NO: 52 - protein, 90 aa, Artificial Sequence
  Synthetic Construct
NIDRPKGLAFTDVDVDSIKIAWESPQGQVSRYRVTYSSPEDGIHELFPAPDGEEDTAELQGLRPGSEYTVSVVALHDDMESQPLIGTQST

SEQ ID NO: 53 - DNA, 276 bp, Artificial Sequence
  Pronectins (13Fn3)
  CDS (1)..(276)
gccatccccgcccccaccgacctgaagttcacccaggtgacccccaccagcctgagcgcccagtggaccccccccaacgtgcagctgaccggctacagagtgagagtgacccccaaggagaagaccggccccatgaaggagatcaacctggcccccgacagcagcagcgtggtggtgagcggcctgatggtggccaccaagtacgaggtgagcgtgtacgccctgaaggacaccctgaccagcagacccgcccagggcgtggtgaccaccctggag

SEQ ID NO: 54 - protein, 92 aa, Artificial Sequence
  Synthetic Construct
AIPAPTDLKFTQVTPTSLSAQWTPPNVQLTGYRVRVTPKEKTGPMKEINLAPDSSSVVVSGLMVATKYEVSVYALKDTLTSRPAQGVVTTLE

SEQ ID NO: 55 - DNA, 264 bp, Artificial Sequence
  Pronectins (14Fn3)
  CDS (1)..(264)
aacgtgagcccccccagaagagccagagtgaccgacgccaccgagaccaccatcaccatcagctggagaaccaagaccgagaccatcaccggcttccaggtggacgccgtgcccgccaacggccagacccccatccagagaaccatcaagcccgacgtgagaagctacaccatcaccggcctgcagcccggcaccgactacaagatctacctgtacaccctgaacgacaacgccagaagcagcgtggtgatcgacgccagcacc

SEQ ID NO: 56 - protein, 88 aa, Artificial Sequence
  Synthetic Construct
NVSPPRRARVTDATETTITISWRTKTETITGFQVDAVPANGQTPIQRTIKPDVRSYTITGLQPGTDYKIYLYTLNDNARSSVVIDAST

SEQ ID NO: 57 - DNA, 270 bp, Artificial Sequence
  Pronectins (15Fn3)
  CDS (1)..(270)
gccatcgacgcccccagcaacctgagattcctggccaccacccccaacagcctgctggtgagctggcagccccccagagccagaatcaccggctacatcatcaagtacgagaagcccggcagcccccccagagaggtggtgcccagacccagacccggcgtgaccgaggccaccatcaccggcctggagcccggcaccgagtacaccatctacgtgatcgccctgaagaacaaccagaagagcgagcccctgatcggcagaaagaagacc

SEQ ID NO: 58 - protein, 90 aa, Artificial Sequence
  Synthetic Construct
AIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 59 - DNA, 264 bp, Artificial Sequence
  Pronectins (16Fn3)
  CDS (1)..(264)
cccggcctgaaccccaacgccagcaccggccaggaggccctgagccagaccaccatcagctgggcccccttccaggacaccagcgagtacatcatcagctgccaccccgtgggcaccgacgaggagcccctgcagttcagagtgcccggcaccagcaccagcgccaccctgaccggcctgaccagaggcgccacctacaacatcatcgtggaggccctgaaggaccagcagagacacaaggtgagagaggaggtggtgaccgtg

SEQ ID NO: 60 - protein, 88 aa, Artificial Sequence
  Synthetic Construct
PGLNPNASTGQEALSQTTISWAPFQDTSEYIISCHPVGTDEEPLQFRVPGTSTSATLTGLTRGATYNIIVEALKDQQRHKVREEVVTV

SEQ ID NO: 61 - DNA, 276 bp, Artificial Sequence
  Adhiron
  CDS (1)..(276)
gccaccggcgtgagagccgtgcccggcaacgagaacagcctggagatcgaggagctggccagattcgccgtggacgagcacaacaagaaggagaacgccctgctggagttcgtgagagtggtgaaggccaaggagcaggtggtggccggcaccatgtactacctgaccctggaggccaaggacggcggcaagaagaagctgtacgaggccaaggtgtgggtgaagccctgggagaacttcaaggagctgcaggagttcaagcccgtgggcgacgcc

SEQ ID NO: 62 - protein, 92 aa, Artificial Sequence
  Synthetic Construct
ATGVRAVPGNENSLEIEELARFAVDEHNKKENALLEFVRVVKAKEQVVAGTMYYLTLEAKDGGKKKLYEAKVWVKPWENFKELQEFKPVGDA

SEQ ID NO: 63 - DNA, 177 bp, Artificial Sequence
  Affibodies
  CDS (1)..(177)
gtggacaacaagttcaacaaggagcagcagaacgccttctacgagatcctgcacctgcccaacctgaacgaggagcagagaaacgccttcatccagagcctgaaggacgaccccagccagagcgccaacctgctggccgaggccaagaagctgaacgacgcccaggcccccaagtga

SEQ ID NO: 64 - protein, 58 aa, Artificial Sequence [sequence omitted]

SEQ ID NO: 65 - DNA, 522 bp, Artificial Sequence
  Affilins (gamma-B Crystallin)
  CDS (1)..(522)
ggcaagatcaccttctacgaggacagagccttccagggcagaagctacgagtgcaccaccgactgccccaacctgcagccctacttcagcagatgcaacagcatcagagtggagagcggctgctggatgatctacgagagacccaactaccagggccaccagtacttcctgagaagaggcgagtaccccgactaccagcagtggatgggcctgagcgacagcatcagaagctgctgcctgatccccccccacagcggcgcctacagaatgaagatctacgacagagacgagctgagaggccagatgagcgagctgaccgacgactgcatcagcgtgcaggacagattccacctgaccgagatccacagcctgaacgtgctggagggcagctggatcctgtacgagatgcccaactacagaggcagacagtacctgctgagacccggcgagtacagaagattcctggactggggcgcccccaacgccaaggtgggcagcctgagaagagtgatggacctgtac

SEQ ID NO: 66 - protein, 174 aa, Artificial Sequence
  Synthetic Construct
GKITFYEDRAFQGRSYECTTDCPNLQPYFSRCNSIRVESGCWMIYERPNYQGHQYFLRRGEYPDYQQWMGLSDSIRSCCLIPPHSGAYRMKIYDRDELRGQMSELTDDCISVQDRFHLTEIHSLNVLEGSWILYEMPNYRGRQYLLRPGEYRRFLDWGAPNAKVGSLRRVMDLY

SEQ ID NO: 67 - DNA, 294 bp, Artificial Sequence
  Affimers
  CDS (1)..(294)
atgatccccagaggcctgagcgaggccaagcccgccacccccgagatccaggagatcgtggacaaggtgaagccccagctggaggagaagaccaacgagacctacggcaagctggaggccgtgcagtacaagacccaggtgctggccagcaccaactactacatcaaggtgagagccggcgacaacaagtacatgcacctgaaggtgttcaacggcccccccggccagaacgccgacagagtgctgaccggctaccaggtggacaagaacaaggacgacgagctgaccggcttc

SEQ ID NO: 68 - protein, 98 aa, Artificial Sequence
  Synthetic Construct
MIPRGLSEAKPATPEIQEIVDKVKPQLEEKTNETYGKLEAVQYKTQVLASTNYYIKVRAGDNKYMHLKVFNGPPGQNADRVLTGYQVDKNKDDELTGF

SEQ ID NO: 69 - DNA, 462 bp, Artificial Sequence
  Anticalin (lipocalin Lcn1)
  CDS (1)..(462)
atcgccagcgacgaggagatccaggacgtgagcggcacctggtacctgaaggccatgaccgtggacagagagttccccgagatgaacctggagagcgtgacccccatgaccctgaccaccctggagggcggcaacctggaggccaaggtgaccatgctgatcagcggcagatgccaggaggtgaaggccgtgctggagaagaccgacgagcccggcaagtacaccgccgacggcggcaagcacgtggcctacatcatcagaagccacgtgaaggaccactacatcttctacagcgagggcgagctgcacggcaagcccgtgagaggcgtgaagctggtgggcagagaccccaagaacaacctggaggccctgctggacttcgagaaggccgccggcgccagaggcctgagcaccgagagcatcctgatccccagacagagcgagacctgcagccccggcagc

SEQ ID NO: 70 - protein, 154 aa, Artificial Sequence
  Synthetic Construct
IASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYSEGELHGKPVRGVKLVGRDPKNNLEALLDFEKAAGARGLSTESILIPRQSETCSPGS

SEQ ID NO: 71 - DNA, 534 bp, Artificial Sequence
  Anticalins (lipocalin Lcn2)
  CDS (1)..(534)
caggacagcaccagcgacctgatccccgccccccccctgagcaaggtgcccctgcagcagaacttccaggacaaccagttccagggcaagtggtacgtggtgggcctggccggcaacgccatcctgagagaggacaaggacccccagaagatgtacgccaccatctacgagctgaaggaggacaagagctacaacgtgaccagcgtgctgttcagaaagaagaagtgcgactactggatcagaaccttcgtgcccggctgccagcccggcgagttcaccctgggcaacatcaagagctaccccggcctgaccagctacctggtgagagtggtgagcaccaactacaaccagcacgccatggtgttcttcaagaaggtgagccagaacagagagtacttcaagatcaccctgtacggcagaaccaaggagctgaccagcgagctgaaggagaacttcatcagattcagcaagagcctgggcctgcccgagaaccacatcgtgttccccgtgcccatcgaccagtgcatcgacggc

SEQ ID NO: 72 - protein, 178 aa, Artificial Sequence
  Synthetic Construct
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 73 - DNA, 255 bp, Artificial Sequence
  Avimers (C426) targeting c-MET
  CDS (1)..(255)
tgcgagagcggcgagttccagtgccacagcaccggcagatgcatcccccaggagtgggtgtgcgacggcgacaacgactgcgaggacagcagcgacgaggcccccgacctgtgcgccagcgccgagcccacctgccccagcggcgagttccagtgcagaagcaccaacagatgcatccccgagacctggctgtgcgacggcgacaacgactgcgaggacggcagcgacgaggagagctgcaccccccccacctga

SEQ ID NO: 74 - protein, 84 aa, Artificial Sequence
  Synthetic Construct
CESGEFQCHSTGRCIPQEWVCDGDNDCEDSSDEAPDLCASAEPTCPSGEFQCRSTNRCIPETWLCDGDNDCEDGSDEESCTPPT

SEQ ID NO: 75 - DNA, 267 bp, Artificial Sequence
  Centyrins (Fn3 domain of Tenascin)
  CDS (1)..(267)
ctgcccgcccccaagaacctggtggtgagcgaggtgaccgaggacagcgccagactgagctggaccgcccccgacgccgccttcgacagcttcctgatcggctacggcgagagcgagaaggtgggcgaggccatcgtgctgaccgtgcccggcagcgagagaagctacgacctgaccggcctgaagcccggcaccgagtacaccgtgagcatctacggcgtgaagggcggccacagaagcaaccccctgagcgccatcttcaccacc

SEQ ID NO: 76 - protein, 89 aa, Artificial Sequence
  Synthetic Construct
LPAPKNLVVSEVTEDSARLSWTAPDAAFDSFLIGYGESEKVGEAIVLTVPGSERSYDLTGLKPGTEYTVSIYGVKGGHRSNPLSAIFTT

SEQ ID NO: 77 - DNA, 171 bp, Artificial Sequence
  Kunitz domain/BPTI
  CDS (1)..(171)
gtgagagaggtgtgcagcgagcaggccgagaccggcccctgcagagccatgatcagcagatggtacttcgacgtgaccgagggcaagtgcgcccccttcttctacggcggctgctgcggcggcaacagaaacaacttcgacaccgaggagtactgcatggccgtgtgcggc

SEQ ID NO: 78 - protein, 57 aa, Artificial Sequence
  Synthetic Construct
VREVCSEQAETGPCRAMISRWYFDVTEGKCAPFFYGGCCGGNRNNFDTEEYCMAVCG

SEQ ID NO: 79 - DNA, 516 bp, Artificial Sequence
  Obodies (human AspRS)
  CDS (1)..(516)
gagatcatggacgccgccgaggactacgccaaggagagatacggcatcagcagcatgatccagagccaggagaagcccgacagagtgctggtgagagtgagagacctgaccatccagaaggccgacgaggtggtgtgggtgagagccagagtgcacaccagcagagccaagggcaagcagtgcttcctggtgctgagacagcagcagttcaacgtgcaggccctggtggccgtgggcgaccacgccagcaagcagatggtgaagttcgccgccaacatcaacaaggagagcatcgtggacgtggagggcgtggtgagaaaggtgaaccagaagatcggcagctgcacccagcaggacgtggagctgcacgtgcagaagatctacgtgatcagcctggccgagcccagactgcccctgcagctggacgacgccgtgagacccgaggccgagggcgaggaggagggcagagccaccgtgaaccaggacaccagactggacaacagagtgatcgacctg

SEQ ID NO: 80 - protein, 172 aa, Artificial Sequence
  Synthetic Construct
EIMDAAEDYAKERYGISSMIQSQEKPDRVLVRVRDLTIQKADEVVWVRARVHTSRAKGKQCFLVLRQQQFNVQALVAVGDHASKQMVKFAANINKESIVDVEGVVRKVNQKIGSCTQQDVELHVQKIYVISLAEPRLPLQLDDAVRPEAEGEEEGRATVNQDTRLDNRVIDL

SEQ ID NO: 81 - DNA, 267 bp, Artificial Sequence
  Tn3A
  CDS (1)..(267)
gccatcgaggtgaaggacgtgaccgacaccaccgccctgatcacctggagcgacgagttcggccacgactacgacggctgcgagctgacctacggcatcaaggacgtgcccggcgacagaaccaccatcgacctgtggtggcacagcgcctggtacagcatcggcaacctgaagcccgacaccgaggacgtgagcctgatctgctacaccgaccaggaggccggcaaccccgccaaggagaccttcaccaccggcctggtgcccaga

SEQ ID NO: 82 - protein, 89 aa, Artificial Sequence
  Synthetic Construct
AIEVKDVTDTTALITWSDEFGHDYDGCELTYGIKDVPGDRTTIDLWWHSAWYSIGNLKPDTEDVSLICYTDQEAGNPAKETFTTGLVPR

SEQ ID NO: 83 - DNA, 276 bp, Artificial Sequence
  Tn3B
  CDS (1)..(276)
gccatcgaggtggaggacgtgaccgacaccaccgccctgatcacctggaccaacagaagcagctacagcaacctgcacggctgcgagctggcctacggcatcaaggacgtgcccggcgacagaaccaccatcgacctgaaccagccctacgtgcactacagcatcggcaacctgaagcccgacaccgagtacgaggtgagcctgatctgcctgaccaccgacggcacctacaacaaccccgccaaggagaccttcaccaccggcctggtgcccaga

SEQ ID NO: 84 - protein, 92 aa, Artificial Sequence
  Synthetic Construct
AIEVEDVTDTTALITWTNRSSYSNLHGCELAYGIKDVPGDRTTIDLNQPYVHYSIGNLKPDTEYEVSLICLTTDGTYNNPAKETFTTGLVPR

SEQ ID NO: 85 - DNA, 177 bp, Artificial Sequence
  Hckomers
  CDS (1)..(177)
accctgttcgtggccctgtacgactacgaggccagaaccgaggacgagctgagcttccacaagggcgagaagttccagatcctgaacagcagcgagggcgactggtgggaggccagagacagcctgaccaccggcgagaccggctacatccccagcaactacgtggcccccgtggac

SEQ ID NO: 86 - protein, 59 aa, Artificial Sequence
  Synthetic Construct
TLFVALYDYEARTEDELSFHKGEKFQILNSSEGDWWEARDSLTTGETGYIPSNYVAPVD

SEQ ID NO: 87 - DNA, 174 bp, Artificial Sequence
  NPHP1
  CDS (1)..(174)
gaggagtacatcgccgtgggcgacttcgacaccgcccagcaggtgggcgacctgaccttcaagaagggcgagatcctgctggtgatcgagaagaagcccgacggctggtggatcgccaaggacgccaagggcaacgagggcctggtgcccagaacctacctggagccctacagc

SEQ ID NO: 88 - protein, 58 aa, Artificial Sequence
  Synthetic Construct
EEYIAVGDFDTAQQVGDLTFKKGEILLVIEKKPDGWWIAKDAKGNEGLVPRTYLEPYS

SEQ ID NO: 89 - DNA, 171 bp, Artificial Sequence
  Tec
  CDS (1)..(171)
gagatcgtggtggccatgtacgacttccaggccgccgagggccacgacctgagactggagagacaggagtacctgatcctggagaagaacgacgtgcactggtggagagccagagacaagtacggcaacgagggctacatccccagcaactacgtgaccggcaagaagtga

SEQ ID NO: 90 - protein, 56 aa, Artificial Sequence
  Synthetic Construct
EIVVAMYDFQAAEGHDLRLERQEYLILEKNDVHWWRARDKYGNEGYIPSNYVTGKK

SEQ ID NO: 91 - DNA, 171 bp, Artificial Sequence
  Hck
  CDS (1)..(171)
atcatcgtggtggccctgtacgactacgaggccatccaccacgaggacctgagcttccagaagggcgaccagatggtggtgctggaggagagcggcgagtggtggaaggccagaagcctggccaccagaaaggagggctacatccccagcaactacgtggccagagtggac

SEQ ID NO: 92 - protein, 57 aa, Artificial Sequence
  Synthetic Construct
IIVVALYDYEAIHHEDLSFQKGDQMVVLEESGEWWKARSLATRKEGYIPSNYVARVD

SEQ ID NO: 93 - DNA, 213 bp, Artificial Sequence
  Amph
  CDS (1)..(213)
tacaaggtggagaccctgcacgacttcgaggccgccaacagcgacgagctgaccctgcagagaggcgacgtggtgctggtggtgcccagcgacagcgaggccgaccaggacgccggctggctggtgggcgtgaaggagagcgactggctgcagtacagagacctggccacctacaagggcctgttccccgagaacttcaccagaagactggac

SEQ ID NO: 94 - protein, 71 aa, Artificial Sequence
  Synthetic Construct
YKVETLHDFEAANSDELTLQRGDVVLVVPSDSEADQDAGWLVGVKESDWLQYRDLATYKGLFPENFTRRLD

SEQ ID NO: 95 - DNA, 192 bp, Artificial Sequence
  RIMBP#3
  CDS (1)..(192)
aagatcatgatcgccgccctggactacgaccccggcgacggccagatgggcggccagggcaagggcagactggccctgagagccggcgacgtggtgatggtgtacggccccatggacgaccagggcttctactacggcgagctgggcggccacagaggcctggtgcccgcccacctgctggaccacatgagc

SEQ ID NO: 96 - protein, 64 aa, Artificial Sequence
  Synthetic Construct
KIMIAALDYDPGDGQMGGQGKGRLALRAGDVVMVYGPMDDQGFYYGELGGHRGLVPAHLLDHMS

SEQ ID NO: 97 - DNA, 180 bp, Artificial Sequence
  IRIKS
  CDS (1)..(180)
cagaaggtgaagaccatcttcccccacaccgccggcagcaacaagaccctgctgagcttcgcccagggcgacgtgatcaccctgctgatccccgaggagaaggacggctggctgtacggcgagcacgacgtgagcaaggccagaggctggttccccagcagctacaccaagctgctggag

SEQ ID NO: 98 - protein, 60 aa, Artificial Sequence
  Synthetic Construct
QKVKTIFPHTAGSNKTLLSFAQGDVITLLIPEEKDGWLYGEHDVSKARGWFPSSYTKLLE

SEQ ID NO: 99 - DNA, 174 bp, Artificial Sequence
  SNX33
  CDS (1)..(174)
ctgaagggcagagccctgtacgacttccacagcgagaacaaggaggagatcagcatccagcaggacgaggacctggtgatcttcagcgagaccagcctggacggctggctgcagggccagaacagcagaggcgagaccggcctgttccccgccagctacgtggagatcgtgaga

SEQ ID NO: 100 - protein, 58 aa, Artificial Sequence
  Synthetic Construct
LKGRALYDFHSENKEEISIQQDEDLVIFSETSLDGWLQGQNSRGETGLFPASYVEIVR

SEQ ID NO: 101 - DNA, 168 bp, Artificial Sequence
  Eps8L1
  CDS (1)..(168)
aagtgggtgctgtgcaactacgacttccaggccagaaacagcagcgagctgagcgtgaagcagagagacgtgctggaggtgctggacgacagcagaaagtggtggaaggtgagagaccccgccggccaggagggctacgtgccctacaacatcctgaccccctacccc

SEQ ID NO: 102 - protein, 56 aa, Artificial Sequence
  Synthetic Construct
KWVLCNYDFQARNSSELSVKQRDVLEVLDDSRKWWKVRDPAGQEGYVPYNILTPYP

SEQ ID NO: 103 - DNA, 177 bp, Artificial Sequence
  FISH#5
  CDS (1)..(177)
gacgtgtacgtgagcatcgccgactacgagggcgacgaggagaccgccggcttccaggagggcgtgagcatggaggtgctggagagaaaccccaacggctggtggtactgccagatcctggacggcgtgaagcccttcaagggctgggtgcccagcaactacctggagaagaagaac

SEQ ID NO: 104 - protein, 59 aa, Artificial Sequence
  Synthetic Construct
DVYVSIADYEGDEETAGFQEGVSMEVLERNPNGWWYCQILDGVKPFKGWVPSNYLEKKN

SEQ ID NO: 105 - DNA, 171 bp, Artificial Sequence
  CMS#1
  CDS (1)..(171)
gtggactacatcgtggagtacgactacgacgccgtgcacgacgacgagctgaccatcagagtgggcgagatcatcagaaacgtgaagaagctgcaggaggagggctggctggagggcgagctgaacggcagaagaggcatgttccccgacaacttcgtgaaggagatcaag

SEQ ID NO: 106 - protein, 57 aa, Artificial Sequence
  Synthetic Construct
VDYIVEYDYDAVHDDELTIRVGEIIRNVKKLQEEGWLEGELNGRRGMFPDNFVKEIK

SEQ ID NO: 107 - DNA, 168 bp, Artificial Sequence
  OSTF1
  CDS (1)..(168)
aaggtgttcagagccctgtacaccttcgagcccagaacccccgacgagctgtacttcgaggagggcgacatcatctacatcaccgacatgagcgacaccaactggtggaagggcaccagcaagggcagaaccggcctgatccccagcaactacgtggccgagcaggcc

SEQ ID NO: 108 - protein, 56 aa, Artificial Sequence
  Synthetic Construct
KVFRALYTFEPRTPDELYFEEGDIIYITDMSDTNWWKGTSKGRTGLIPSNYVAEQA

SEQ ID NO: 109 - protein, 34 aa, Artificial Sequence
  Cysknots/Knottin
CSPSGAICSGFGPPEQCCSAGCVLNRRARSWRCQ

SEQ ID NO: 110 - protein, 35 aa, Artificial Sequence
  Cysknots/Knottin (SOTI-III)
CSPSGAICSGFGPPEQCCSAGACVPHPILRIFVCQ

SEQ ID NO: 111 - protein, 29 aa, Artificial Sequence
  Kalata B1
GLPVCGETCVGGTCNTPGCTCSWPVCTRN

SEQ ID NO: 112 - protein, 29 aa, Artificial Sequence
  Kalata B2
GLPVCGETCFGGTCNTPGCSCTWPICTRD

SEQ ID NO: 113 - protein, 34 aa, Artificial Sequence
  MCoTI-I
GGVCPKILQRCRRDSDCPGACICRGNGYCGSGSD

SEQ ID NO: 114 - protein, 34 aa, Artificial Sequence
  MCoTI-II
GGVCPKILKKCRRDSDCPGACICRGNGYCGSGSD

SEQ ID NO: 115 - DNA, 63 bp, Artificial Sequence
  Murine CD8 Transmembrane domain
atctgggcacccttggccggaatctgcgtggcccttctgctgtccttgatcatcactctcatc

SEQ ID NO: 116 - DNA, 81 bp, Artificial Sequence
  Murine CD28 Transmembrane domain
ttttgggcactggtcgtggttgctggagtcctgttttgttatggcttgctagtgacagtggctctttgtgttatctggaca

SEQ ID NO: 117 - DNA, 135 bp, Artificial Sequence
  Murine 4-1BB Intracellular domain
aaatggatcaggaaaaaattcccccacatattcaagcaaccatttaagaagaccactggagcagctcaagaggaagatgcttgtagctgccgatgtccacaggaagaagaaggaggaggaggaggctatgagctg

SEQ ID NO: 118 - DNA, 123 bp, Artificial Sequence
  Murine CD28 Intracellular domain
aatagtagaaggaacagactccttcaaagtgactacatgaacatgactccccggaggcctgggctcactcgaaagccttaccagccctacgcccctgccagagactttgcagcgtaccgcccc

SEQ ID NO: 119 - DNA, 336 bp, Artificial Sequence
  Murine CD3zeta Intracelllular domain
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccct

SEQ ID NO: 120 - DNA, 19 bp, Artificial Sequence
  MLV-U3 forward primer
agcccacaacccctcactc

SEQ ID NO: 121 - DNA, 18 bp, Artificial Sequence
  MLV-Psi Reverse primer
tctcccgatcccggacga

SEQ ID NO: 122 - DNA, 26 bp, Artificial Sequence
  Amplificaiton Probe
ccccaaatgaaagacccccgctgacg

(19) United States
(12) Patent Application Publication
(10) Pub. No.: US 2022/0090132 A1
(43) Pub. Date: Mar. 24, 2022

(54) CAR T CELL METHODS AND CONSTRUCTS

(71) Applicant: Abintus Bio, Inc., San Diego, CA (US)

(72) Inventors: Douglas J. Jolly, Encinitas, CA (US); Joan M. Robbins, San Diego, CA (US); Amy H. Lin, San Diego, CA (US); Derek G. Ostertag, San Diego, CA (US); Cornelia Bentley, San Diego, CA (US); Sophie Viaud, San Francisco, CA (US)

(21) Appl. No.: 17/419,239

(22) PCT Filed: Jan. 6, 2020
(86) PCT No.: PCT/US2020/012417

§ 371 Date: Jun. 28, 2021

Related U.S. Application Data
(60) Provisional application No. 62/788,894, filed on Jan. 6, 2019.

(51) Int. Cl. C12N 15/86; C12N 7/00; C07K 14/705; C07K 14/725; C07K 16/28

(52) U.S. Cl. CPC C12N 15/86; C12N 7/00; C07K 14/70521; C07K 14/70517; A61K 2039/505; C07K 14/70578; C07K 16/2803; C12N 2740/10043; C12N 2740/10071; C07K 14/7051

(57) ABSTRACT

The disclosure provides non-replicating viral vectors (RNV) for adoptive cell therapy. The RNVs can deliver chimeric antigen receptors to immune cells (e.g., T-Cells) in vivo.

### CROSS REFERENCE TO RELATED APPLICATIONS

The application claims priority under 35 U.S.C. § 119 to U.S. Provisional Application Ser. No. 62/788,894, filed Jan. 6, 2019, the disclosures of which are incorporated herein by reference.

### TECHNICAL FIELD

The disclosure provides replicating viral vectors (RRVs) and non-replicating viral vectors (RNVs) for adoptive cell therapy. The RRVs and RNVs can deliver chimeric antigen receptors to immune cells (e.g., T-Cells) in vivo.

### INCORPORATION BY REFERENCE OF SEQUENCE LISTING

Accompanying this filing is a Sequence Listing entitled “Sequence-Listing ST25.txt”, created on Jan. 6, 2020 and having 172,643 bytes of data, machine formatted on IBM-PC, MS-Windows operating system. The sequence listing is hereby incorporated herein by reference in its entirety for all purposes.

### BACKGROUND

In 2013 Science magazine named Cancer Immunotherapy as “breakthrough of the year” (J. Couzan-Frankel, Science, 342:1432-1433, 2013). Chimeric antigen receptor (CAR)-engineered T cells (June & Sadelain, N Engl J Med, 379:64-73, 2018) were a key part of this designation with at least two groups of researchers, beginning in 2013, reporting on eye-catching responses to CAR therapy in patients, with “pounds of leukemia that melted away,” and showing amazing recoveries from advanced cancers. CARs are engineered receptors that graft a defined specificity onto an immune effector cell, typically a T cell, that augments T-cell function. Recently two forms of this therapy, Kymriah (also known as tisagenlecleucel, [www.]hcp.novartis.com/products/kymriah/, marketed by Novartis) and Yeskarta (also known as axicabtagene ciloleucel, [www.]yescartahcp.com developed by Kite Pharmaceuticals, now marketed by Gilead) have been approved for commercial sale, for various types of B cell leukemias, representing a breakthrough in personalized medicine. In this strategy, a patient's own T lymphocytes are harvested, cultured ex vivo, and genetically modified to encode a synthetic receptor that binds a tumor antigen, allowing T cells to recognize and kill antigen-expressing cancer cells after reinfusion into the patient. This approach, as noted above, has been approved for CARs recognizing the B cell marker CD19 (S. L. Maude et al., N Engl J Med., 371:1507-17, 2014) and has demonstrated success clinically for CD20 and BCMA, both markers for various types of lymphoma as well as some types of normal B cells, for which deficient patients can be simply treated by infusions of gamma-globulins. There are now active efforts underway to use these types of therapies against solid tumors (K. B. Long et al., Front. Immunol., 2018, [https://]doi.org/10.3389/fimmu.2018.02740; K. Ullah et al., Oncol. Reports, 2018; [https://]doi.org/10.3892/or.2018.6758); to modify different populations such as CD34⁺ hematopoietic stem cells, gamma delta T cells and NK cells; to use other types of genetic modifications of blood/bone marrow, spleen cells to achieve anticancer, antiviral and other types of therapeutic effects. A good example is CD34⁺ hematopoietic stem cells for which there is an approved therapy (Strimvelis™, developed by GlaxoSmithKline, now marketed by Orchard Therapeutics) in Europe for treatment of children with the genetic disease, X-linked Severe Combined Immunodeficiency Syndrome (X-SCIDS). The vast majority of these methods involve the isolation ex vivo of patient's cells, genetic modification of the susceptible cell population and re-implantation. One of the earliest examples of this strategy was the partially successful treatment of ADA-SCIDS patients (R. M. Blaese, Immunol. Res., 38:274-284, 2007; DOI 10.1007/s12026-007-0009-z) and attempts to treat graft versus host disease after donor lymphocyte infusion as part of allogeneic bone marrow transplant treatment (G. Bonini, Science, 276:1719-1724, 1997). All of these procedures involved ex vivo genetic modifications of patient T cells with retroviral vectors, lentiviral vectors or other gene transfer methods followed by reinfusion of the modified cells.

The above-discussed therapies are all performed with ex vivo cell preparations, usually with autologous tissues. There are several reasons for this approach: firstly the gene transfer agents used have not been very efficient and it was only possible to transduce the target cells by using them with partially or completely purified preparation; secondly, injected gene transfer vectors, particularly integrating vectors, were perceived to be potentially risky for insertional mutagenesis, as such procedures have led to lymphomas in some immune-suppressed individuals, for example in the X-SCIDS patients; thirdly the ex vivo approach was also seen as safer from the point of view that it would be easier to identify abnormal cells or other abnormalities in an ex vivo preparation of cells. This ex vivo transduction strategy has proved to be an extremely limiting factor for: therapy availability; cost; and consistency ([https://]xtalks.com/kymriah-manufacturing-issues-spell-trouble-for-commercialization-of-novartis-car-t-therapy-1475/, [https://]l abiotech.eu/medical/kymriah-car-t-therapy-novartis-sales/). Further drawbacks to the ex vivo approaches were the variation in the starting material leading to lack of predictability and timing of success, the extreme laboriousness and expense of the procedure with fresh product prepared for each patient, and the need in most cases to create “room” for successful eventual re-implantation by ablation of existing cell populations in the patient (e.g., S. L. Maude et al., N Engl J Med., 378:439-448, 2018; doi: 10.1056/NEJMoa1709866). The use of engineered allogeneic T cells has been proposed as a solution and clinical trials have started, but there are several issues attached to this stop-gap methodology. Firstly, there are essentially no perfect HLA matches except between identical twins, so the CAR T cells will likely be short-lived an eliminated by an allogeneic response; attempts to use cells where the HLA has been removed increases the risk of a leukemia derived from CAR T cells. Secondly, although this approach potentially allows treatment of more than a single patient with a particular preparation of allogeneic CAR T cells, the maximum number of patients that can be treated is estimated as about 100; then a fresh preparation of unknown potency will need to be made and tested in vitro, which can be quite misleading for in vivo potency prediction.

### SUMMARY

The disclosure provides a way around at least some of these difficulties by using direct administration of integrating gene transfer vectors encoding therapeutic gene(s) that transduce patient's cells directly. In one embodiment the vector is a retroviral vector, the target is a T cell and the transgene is a CAR or similar artificial receptor construct (e.g., 1st, 3^{rd} etc. generation CAR constructs). In another embodiment, the vector is retroviral vector, the target is a cell that has been activated by administration of an external agent and the transgene encodes a therapeutic activity. In this and other embodiments a retroviral vector can be replicating (RRV) or non-replicating (RNV) and can be derived from any integrating virus such as a foamy virus, a lentivirus, an alpha, beta or gamma retrovirus or CRISPR elements and also includes non-viral integrating vector such as those based on transposons such as “Piggy-bac” (Saito et al., Cytother. 16:1257-1269, 2014) or “Sleeping Beauty”. In another embodiment, the vector is a retroviral vector, the activating agent is granulocyte colony stimulating factor (GCSF) and the target is CD34⁺ cells. In a further embodiment, the vector is a gamma retroviral non-replicating vector, the target population is naturally activated T cells, and the transgene is a T cell receptor. In a further embodiment, the vector is a gamma retroviral vector, the target cell population is a naturally activated cell population and the transgene encodes an immune activating agent. In yet another embodiment, the vector is a gamma retroviral vector, the target population is naturally activated T cells, and the transgene is a CAR (e.g., 1^{st}, 2^{nd}, 3^{rd} generation CAR). In a further embodiment, the CAR comprises a binding domain that targets one or more of: CDS; CD19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CA1X); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Ab1) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDG1cp(1-1)Cer); transglutaminase 5 (TGSS); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTI); Cancer/testis antigen 1 (NY-ES0-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P4501B 1 (CYP1B 1); CCCTC-Binding Factor (Zinc Finger Protein)-Like (BORIS or Brother of the Regulator ofImprinted Sites), Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRD; Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLU), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GM1, PTK7, gpNMB, CDH1-CD324, DLL3, CD276/B7H3, IL11Ra, IL13Ra2, CD179b-IGL11, TCRgamma-delta, NKG2D, CD32 (FCGR2A), Tn ag, Tim1-/HVCR1, CSF2RA (GM-CSFR-alpha), TGFbetaR2, Lews Ag, TCR-betal chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLV1-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRCSD, Claudin18.2 (CLD18A2 or CLDN18A.2)), P-glycoprotein, STEAP1, Liv1, Nectin-4, Cripto, gpA33, BST1/CD157, low conductance chloride channel, and the antigen recognized by TNT antibody.

### BRIEF DESCRIPTION OF THE FIGURES

FIG. 1 shows the sequence and annotation of the sequence of a plasmid construct of the disclosure.

FIG. 2 shows the viral RNA (vRNA) sequence with the multiple cloning site highlighted/bold/underlined. A CAR coding sequence (RNA) can be cloned into the multiple cloning site (MCS) to form the RNVCAR viral sequence.

FIG. 3 shows various DNA sequences encoding CARs used to exemplify the invention. Note that the corresponding RNA sequence is contemplated wherein T is replaced with U in the sequences.

FIG. 4 shows various plasmid sequences containing an scFv sequence that binds to CD19 for the production of an CD19-RNVCAR.

FIG. 5A-B schematically depicts exemplary RNV vectors of the disclosure.

FIG. 6 a plot of Infection level in PBMC in Balb/c mice with RNV-GFP determined in peripheral blood mononuclear cells (PBMC) at 7 days after the first IV dose of RNV-GFP.

FIG. 7 shows a plot of RNV-GFP infection evaluated in a subset of PBMC populations: CD11b⁺, CD4⁺, CD8⁺, and CD19⁺ in parallel samples to those taken to generate the data for FIG. 6.

FIG. 8 shows the results of an experiment in theA20 B-cell lymphoma animal model where tumor-bearing mice injected IV with the vector, RNV-1D3CAR, at a dose of 1E7 or 1E8 TU per day for five consecutive days, starting at day 5 after A20 implantation. Mice were monitored for 37 days and survival assessed. The higher dose of RNV-1D3CAR (5E8 TU) led to an improvement in survival in A20 lymphoma tumor bearing mice compared to a vehicle treated control group (no RNV) or the lower dose of RNV-1D3CAR (5E7).

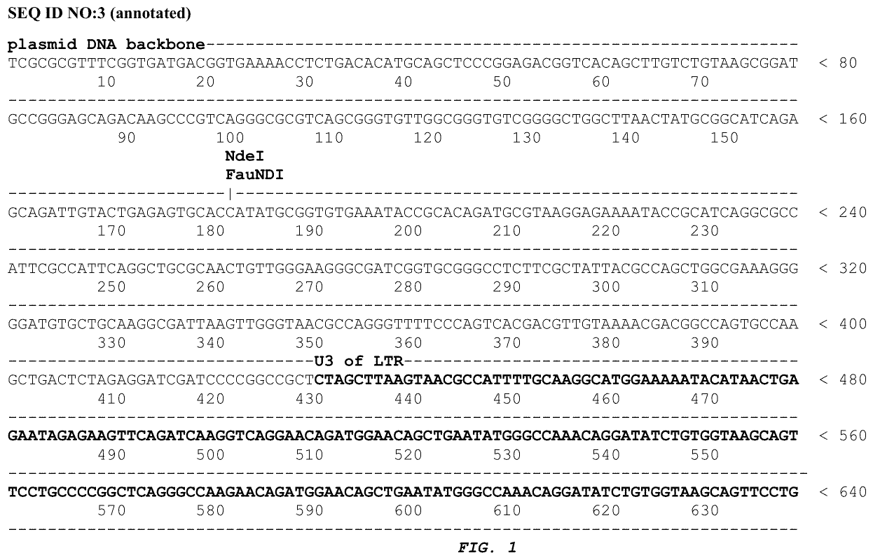

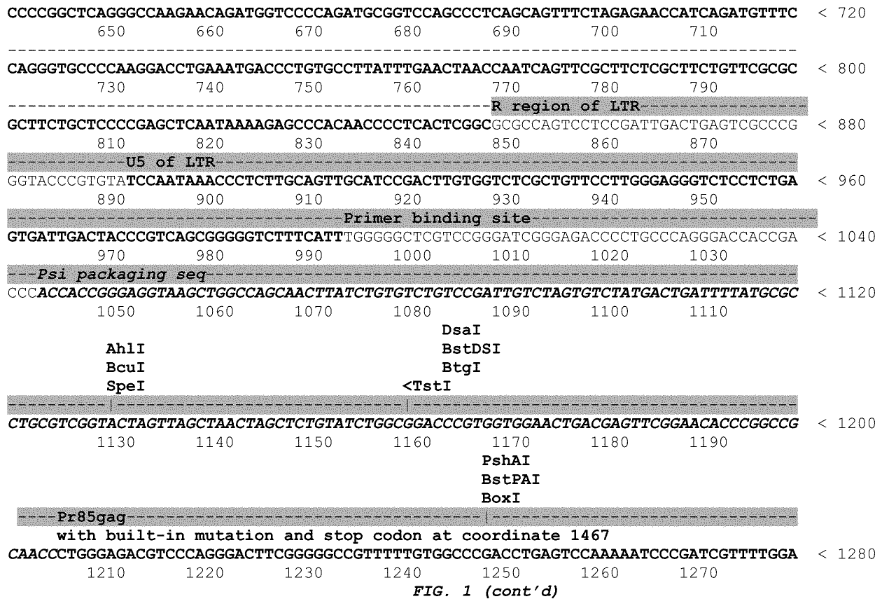

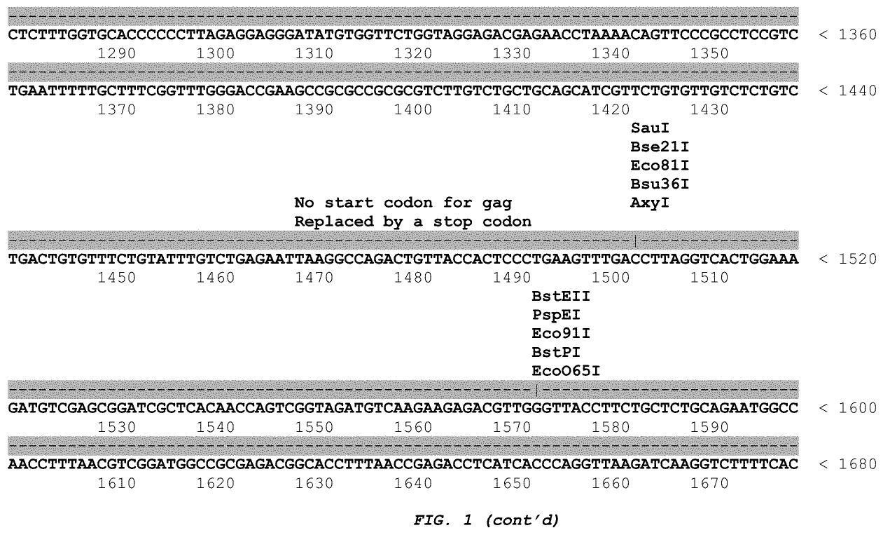

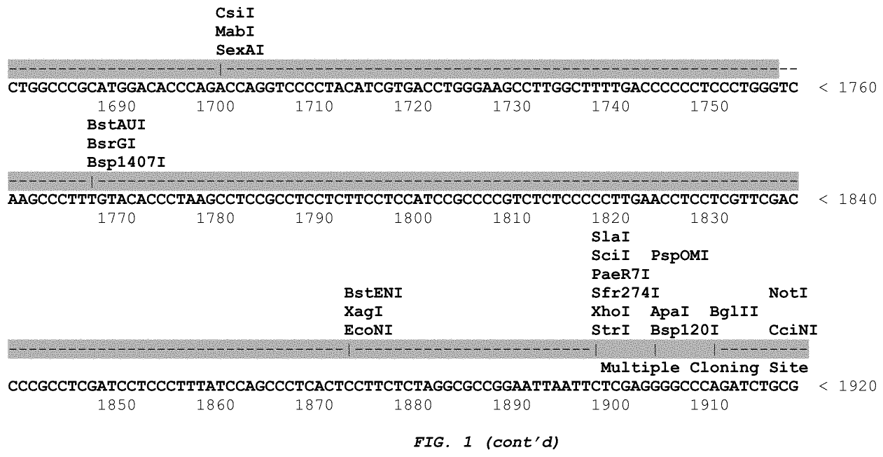

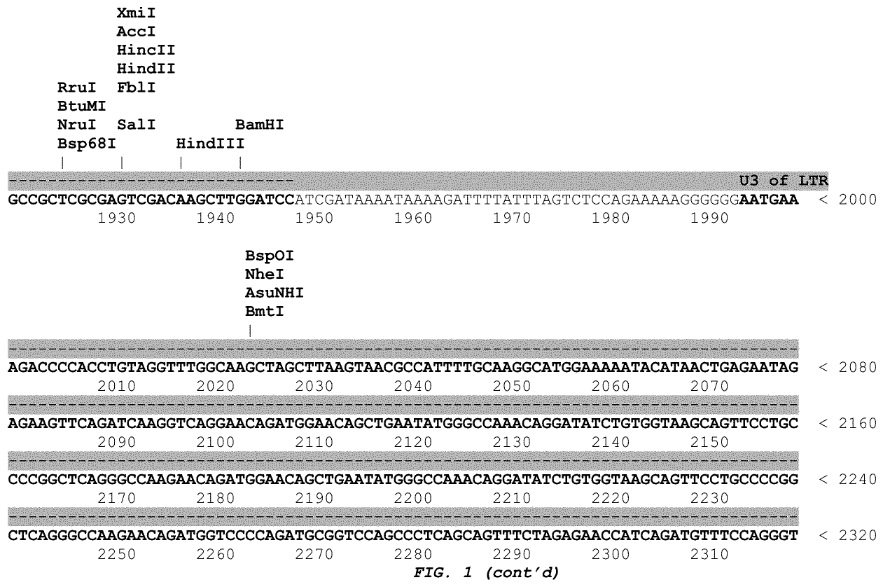

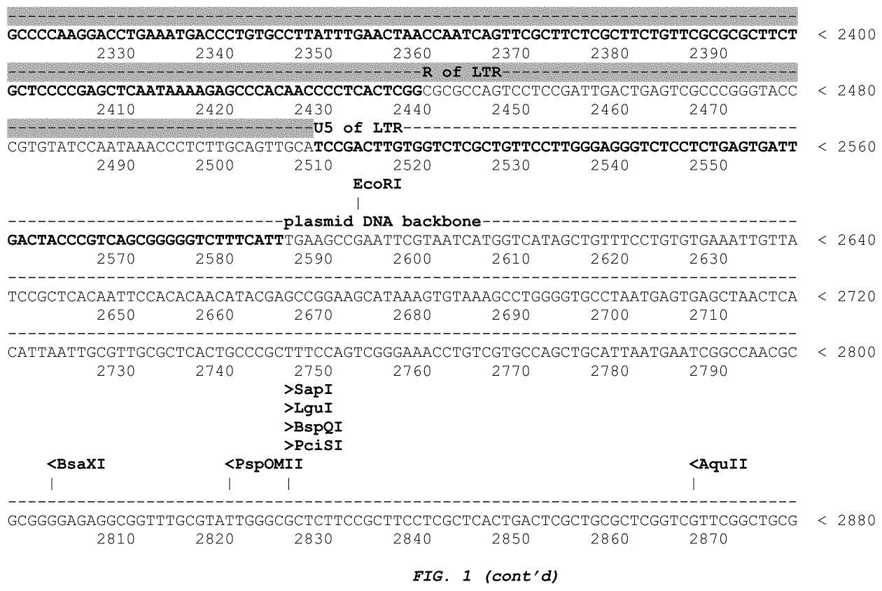

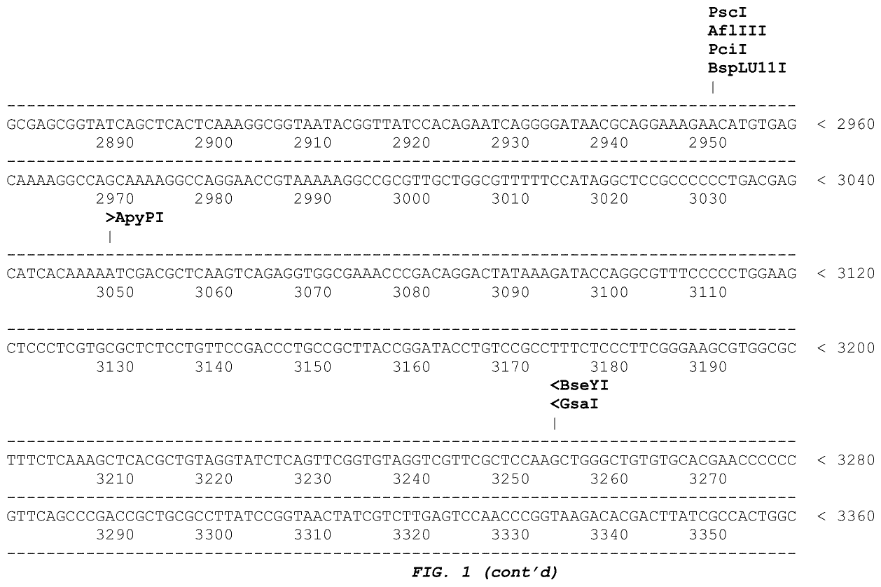

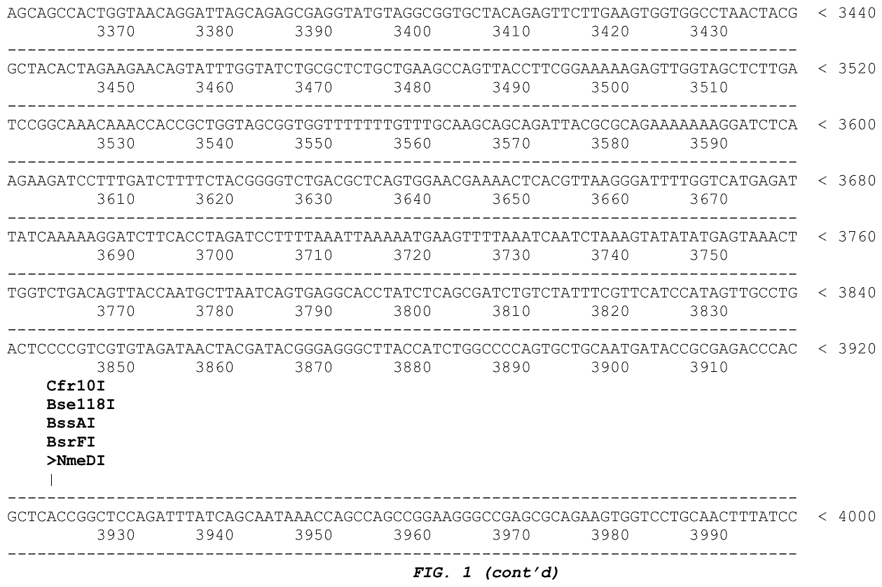

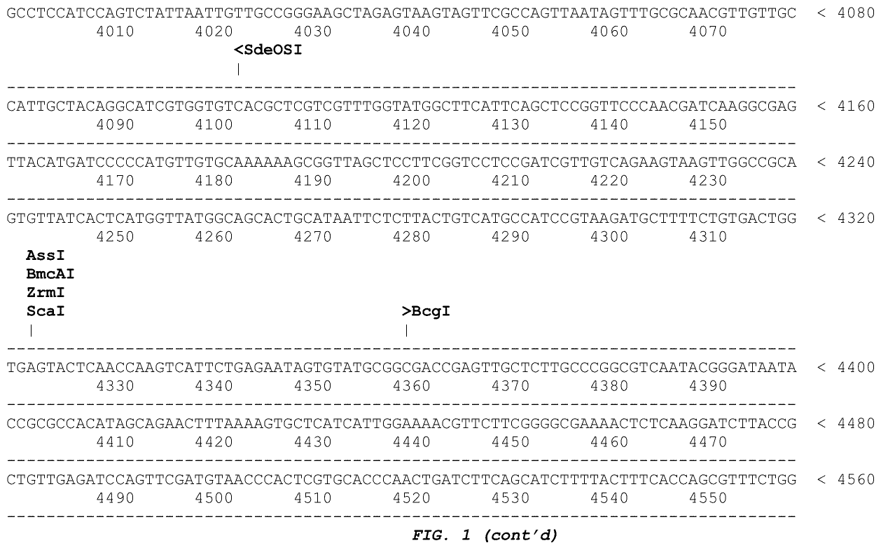

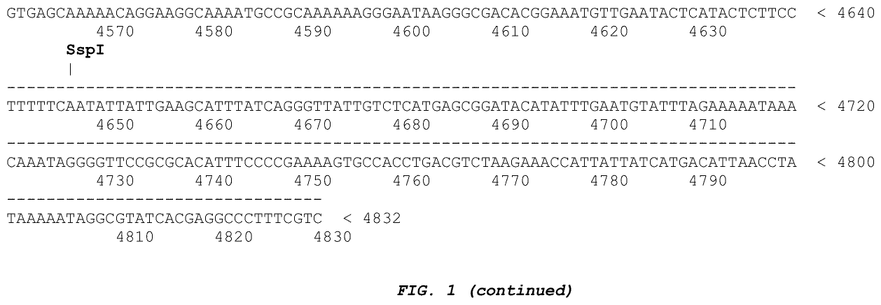

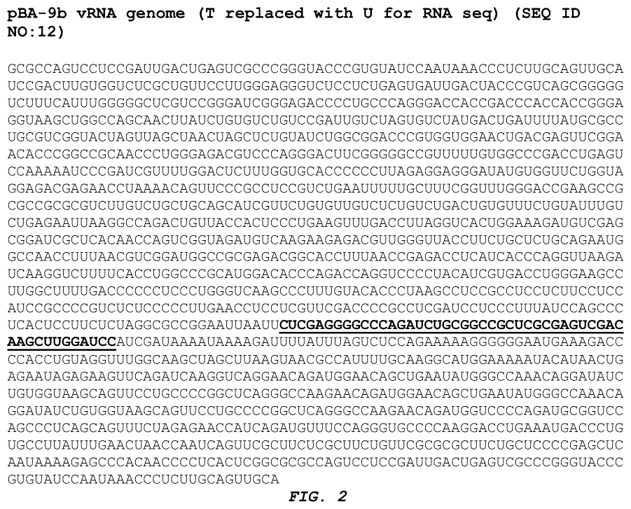

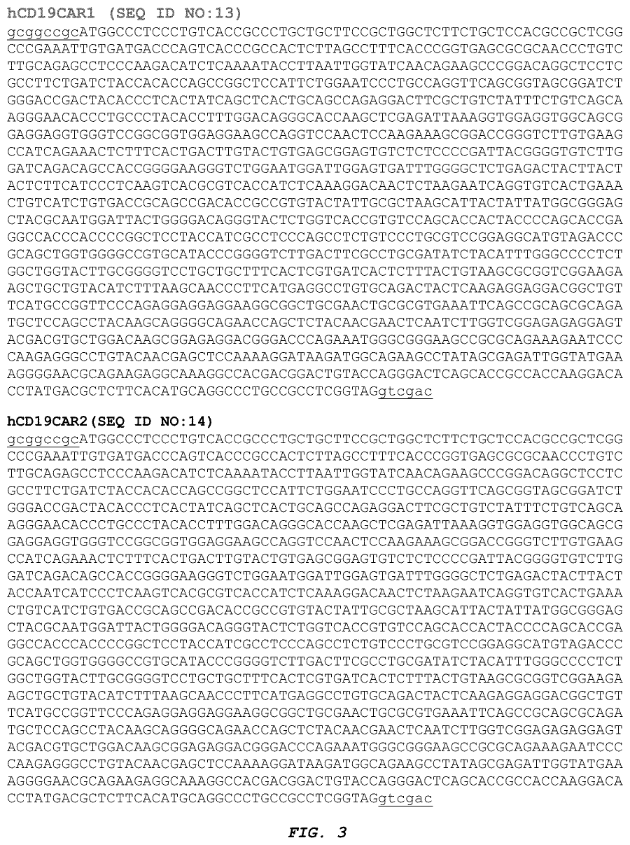

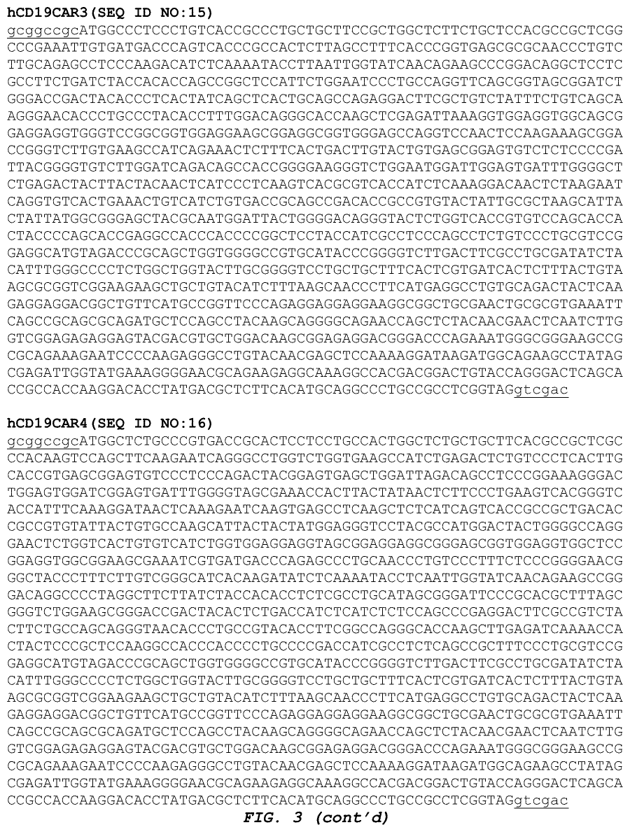

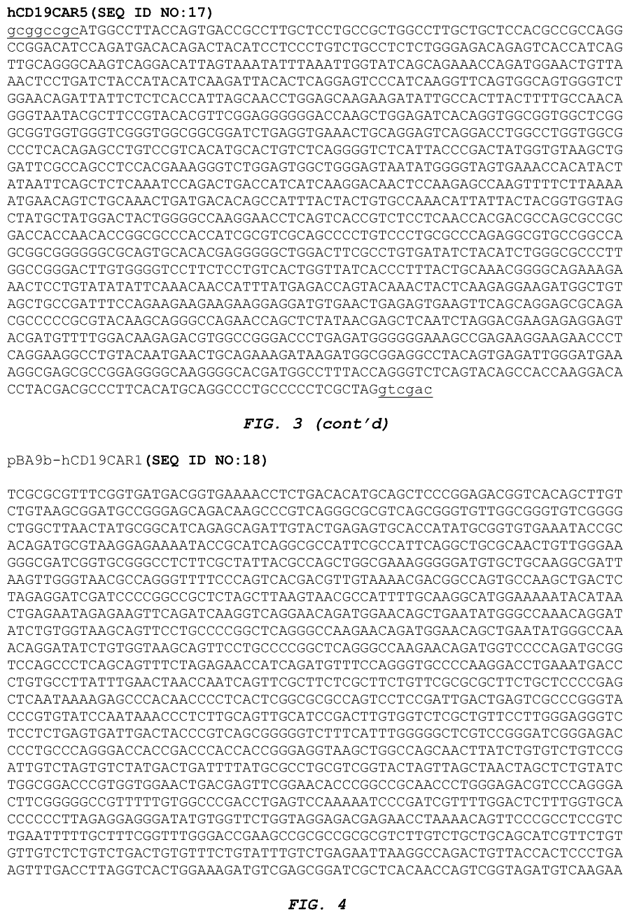

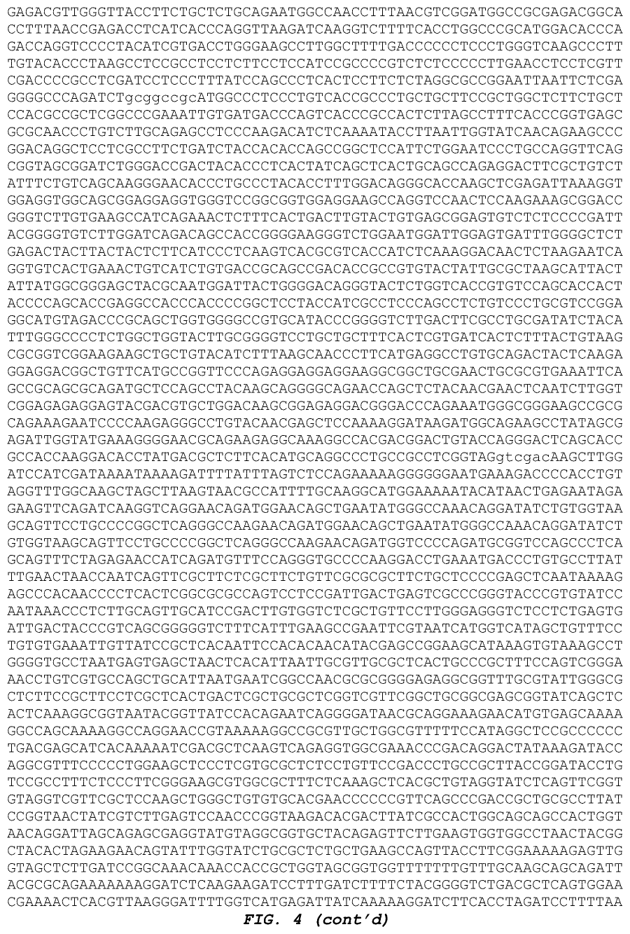

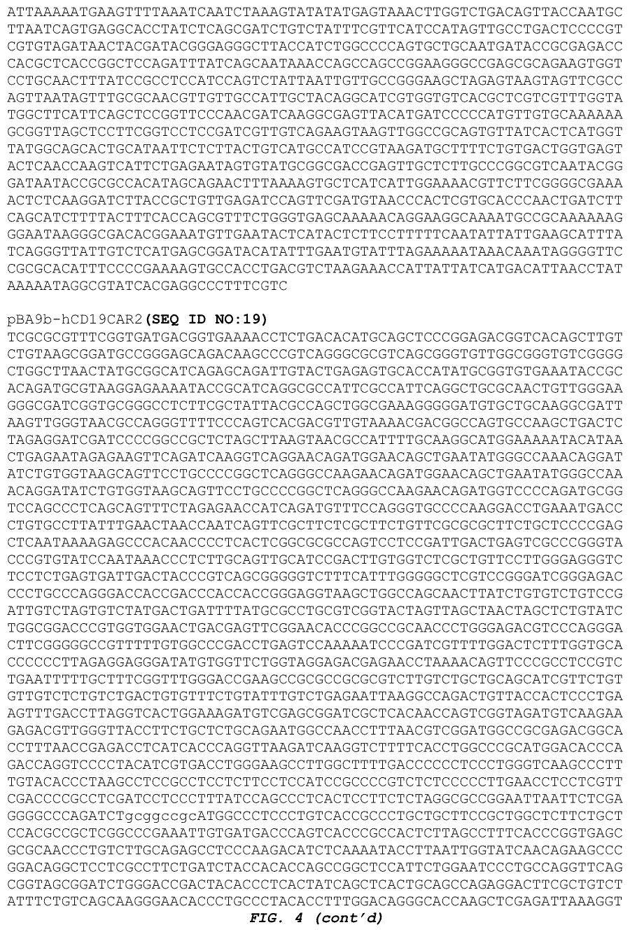

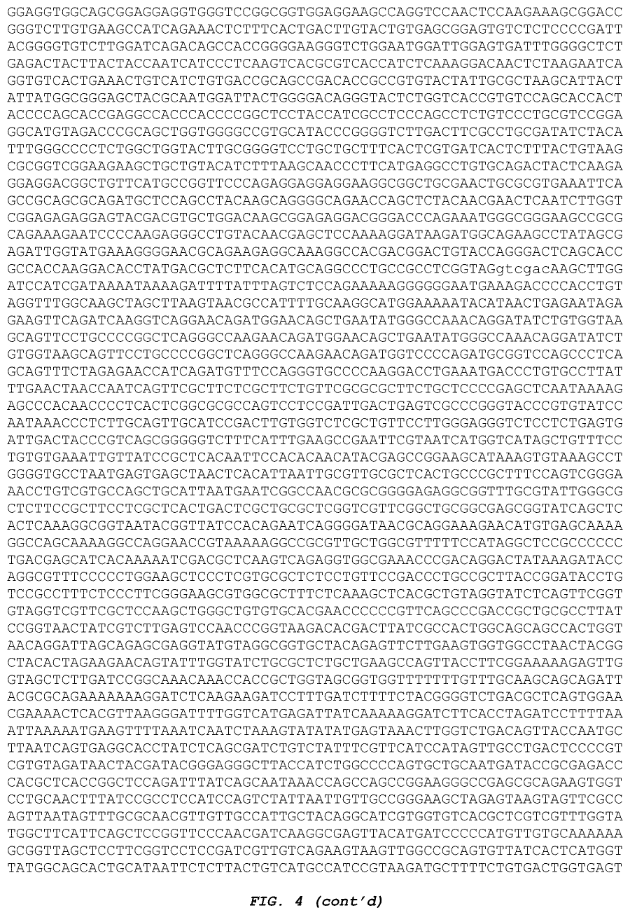

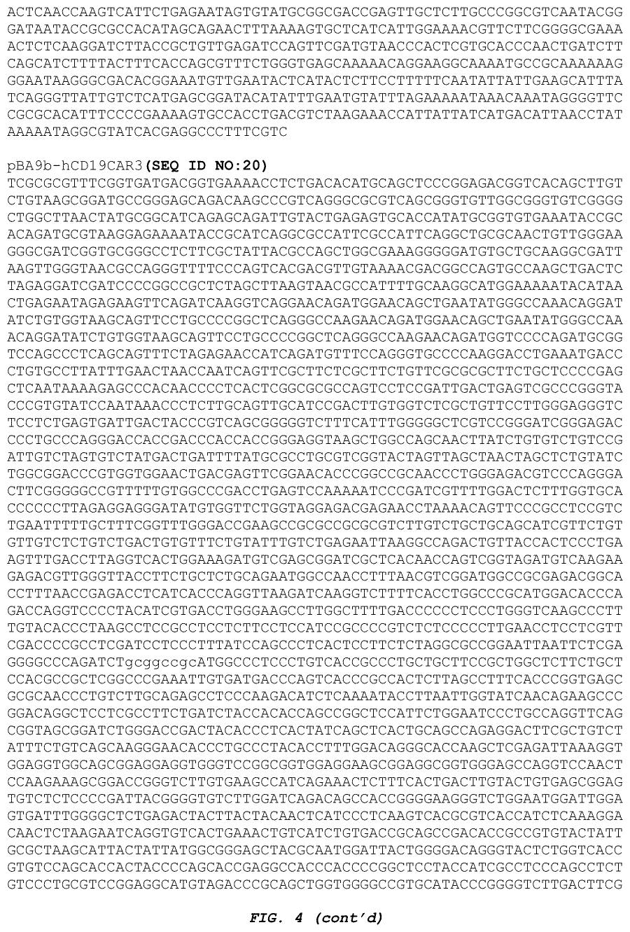

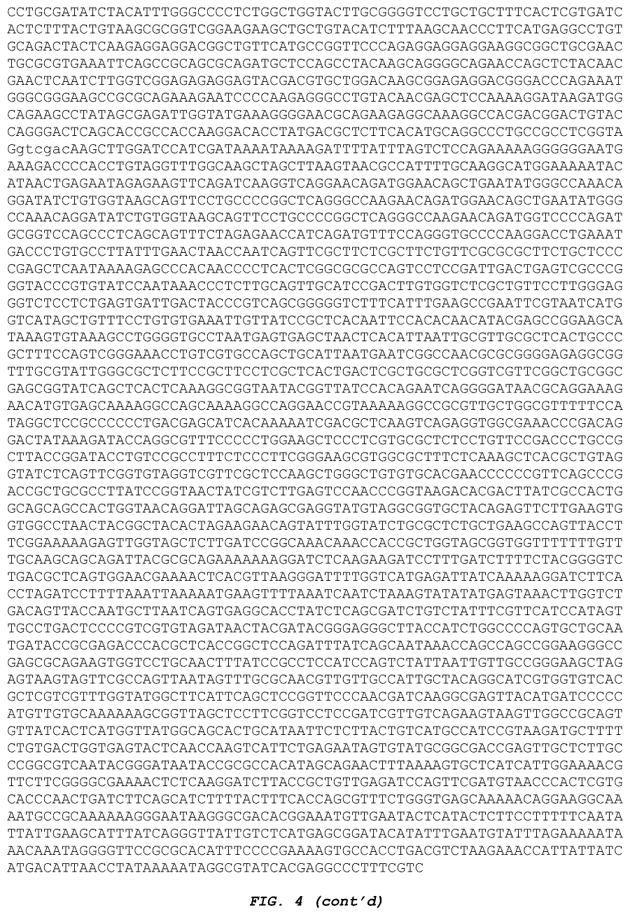

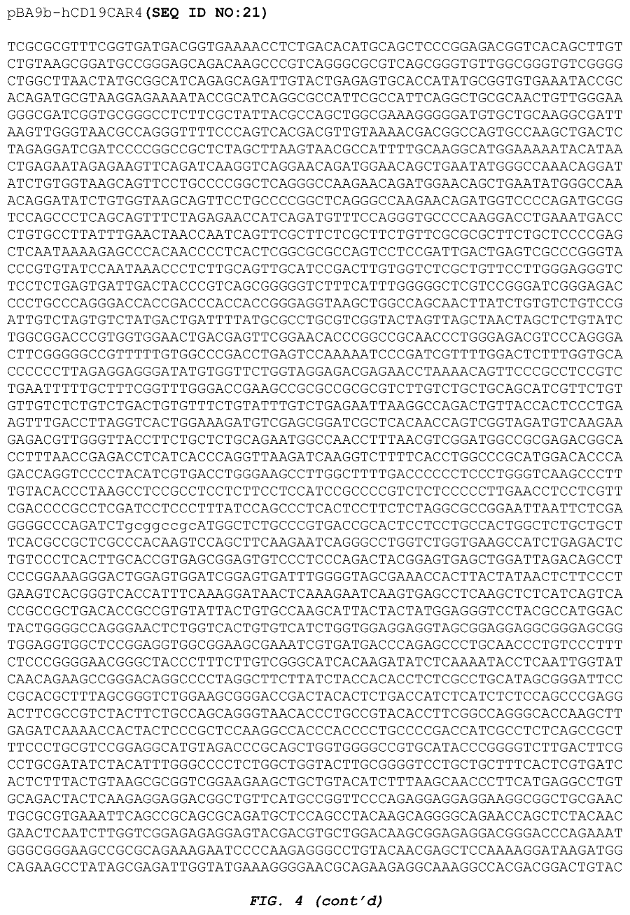

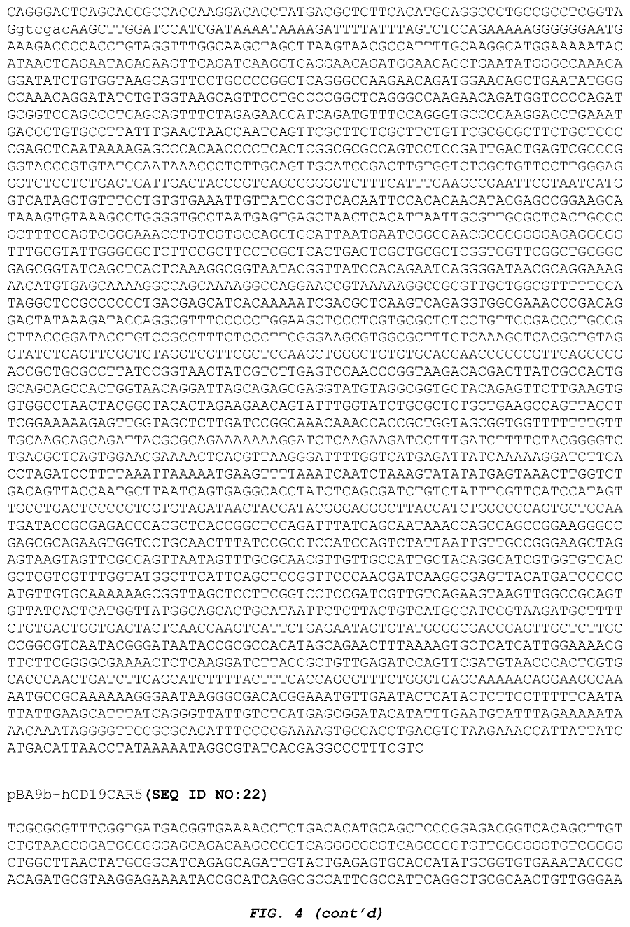

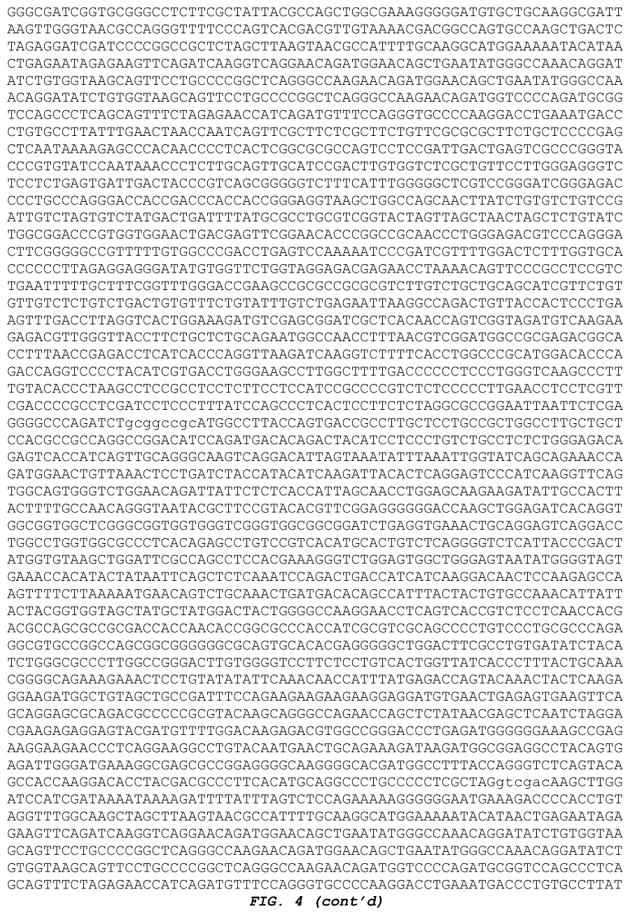

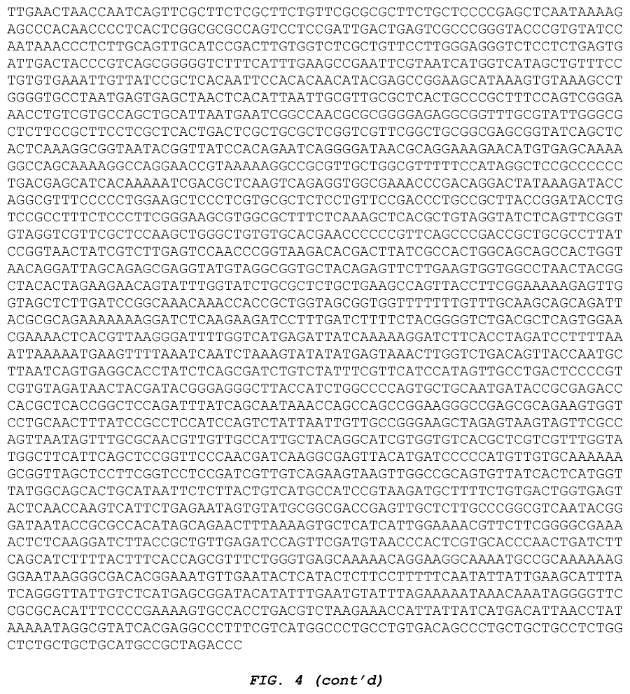

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the polynucleotide” includes reference to one or more polynucleotides and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless otherwise defined, all technical and scientific terms used herein have the same meaning as commonly understood by one of ordinary skill in the art to which this invention belongs. Allen et al., Remington: The Science and Practice of Pharmacy 22nd ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Kohler and Milstein, Derivation of specific antibody-producing tissue culture and tumor lines by cell fusion, Eur. J. Immunol. 1976 Jul. 6(7):511-9; Queen and Selick, Humanized immunoglobulins, U. S. Patent No. 5,585,089 (1996 Dec); and Riechmann et al., Reshaping human antibodies for therapy, Nature 1988 Mar. 24, 332(6162):323-7. Alternatively, monoclonal antibodies or other binding molecules to provide the binding portion of T cell activating molecule of a CAR can be prepared and provided by commercial suppliers All headings and subheading provided herein are solely for ease of reading and should not be construed to limit the invention. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and specific examples are illustrative only and not intended to be limiting.

The disclosure provides pharmaceutical preparations, vectors, cells and methods for use in adoptive cell therapy. The vectors of the disclosure comprise a replicating or non-replicating viral-derived vector comprising coding sequences for various chimeric antigen receptors.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods or describe the compositions herein. Moreover, any value or range (e.g., less than 20 or similar terminology) explicitly includes any integer between such values or up to the value. Thus, for example, “one to five mutations” explicitly includes 1, 2, 3, 4, and/or 5 mutations.

The term “antigen binding molecule” (ABM) refers to any molecule which has a specific affinity for a target antigen as demonstrated in a binding assay such as an ELISA. It includes antibodies, antibody fragments, camelid or shark or other atypically structured antibodies and also a wide range of antibody substitutes or surrogates such as non-immunoglobulin (Ig) scaffold proteins. Such molecules have been developed for biotherapeutics using randomization strategies to identify antigen-binding sequences (U. H Wiedle et al. Cancer Genomics & Proteomics 10: 155-168, 2013; K. Skrlec et al. Trends in Biotechnology,33:408-418, 2015). Non-Ig scaffold proteins are domain-derived subunit of natural proteins from human and other species or are artificial and their size range from 6-20 kDa and can be expressed from a single polypeptide. They possess surface-exposed loops or amino acids in alpha-helical or beta sheet framework that can tolerate insertion, deletion and substitutions which via randomization, phage display screening and affinity maturation processes resulted in antigen-binding scaffold proteins that can function as antagonists or agonists. To date, there are more than 50 different classes of non-Ig scaffold proteins that have been identified and developed for therapeutics as scaffold binders. Due to their size, one major challenge these proteins face are fast renal clearance leading to short half-life in circulation but this not an issue when they are incorporated into a CAR or other membrane penetrating molecule.

A separate challenge is that they often have lower binding affinity (KD 1-100 nM) than monoclonal antibodies and are associated with fast dissociation rates. Genetic modification of these scaffold proteins to include dimerization domain may increase steric hindrance-mediated blocking or avidity so that in certain signaling pathways this can lead to biological functions and therapeutic effects. Multiple methods have been proposed and at least partially tested using fusion proteins containing scaffold proteins.

The disclosure provides compositions and methods that use binding domains that comprise combinations of heavy and/or light chain CDRs linked by scaffold domains (e.g., Adhiron scaffold; scaffolds from human stefin A—see, EP22792058B1 and WO2019/008335 the disclosures of which are incorporated herein by reference). Tables 1 and 2 provide sequences useful in the compositions and methods of the disclosure. Please note that “T” can be “U” in the following nucleic acid sequences as RNA is contemplated.

TABLE 1
Amino acid sequence of some non-Ig
scaffold proteins that can
function as antigen-binding proteins:

| SCAFFOLD | Amino Acid Sequence (SEQ ID NO:) |
|---|---|
| Adnectins | VSDVPRKLEVVAATPTSLLISWDAPAVTVRYY |
| (10Fn3) | RITYGETGGNSPVQEFTVPGSKSTATISGLKP |
| | GVDYTITVYAVTGRGDSPASSKPISNYRTALE |
| | (SEQ ID NO: 24) |
| Adenectin 1 | VSDVPRKLEVVAATPTSLLISWDSGRGSYRYY |
| | RITYGETGGNSPVQEFTVPGPVHTATISGLKP |
| | GVDYTITVYAVTDHKPHADGPHTYHESPISNY |
| | RTALE (SEQ ID NO: 26) |
| Adenectin 2 | VSDVPRKLEVVAATPTSLLISWEHDYPYRRYY |
| | RITYGETGGNSPVQEFTVPKDVDTATISGLKP |
| | GVDYTITVYAVTSSYKYDMQYSPISNYRTALE |
| | (SEQ ID NO: 28) |
| Pronectins | SGPVEVFITETPSQPNSHPIQWNAPQPSHISK |
| (1Fn3) | YILRWRPKNSVGRWKEATIPGHLNSYTIKGLK |
| | PGVVYEGQLISIQQYGHQEVTRFDFTTT |
| | (SEQ ID NO: 30) |
| Pronectins | SPLVATSESVTEITASSFVVSWVSASDTVSGF |
| (2Fn3) | RVEYELSEEGDEPQYLDLPSTATSVNIPDLLP |
| | GRKYIVNVYQ1SEDGEQSLILSTSQTT |
| | (SEQ ID NO: 32) |
| Pronectins | APDAPPDPTVDQVDDTSIVVRWSRPQAPITGY |
| (3Fn3) | RIVYSPSVEGSSTELNLPETANSVTLSDLQPG |
| | VQYNITIYAVEENQESTPVVIQQETTGTPR |
| | (SEQ ID NO: 34) |
| Pronectins | TVPSPRDLQFVEVTDVKVTIMWTPPESAVTGY |
| (4Fn3) | RVDVIPVNLPGEHGQRLPISRNTFAEVTGLSP |
| | GVTYYFKVFAVSHGRESKPLTAQQTT |
| | (SEQ ID NO: 36) |
| Pronectins | KLDAPTNLQFVNETDSTVLVRWTPPRAQITGY |
| (5Fn3) | RLTVGLTRRGQPRQYNVGPSVSKYPLRNLQPA |
| | SEYTVSLVAIKGNQESPKATGVFTTL |
| | (SEQ ID NO: 38) |
| Pronectins | QPGSSIPPYNTEVTETTIVITWTPAPR1GFKL |
| (6Fn3) | GVRPSQGGEAPREVTSDSGSVVSGLTPGVEYV |
| | YTIQVLRDGQERDAPIVNKVVT |
| | (SEQ ID NO: 40) |
| Pronectins | PLSPPTNLHLEANPDTGVLTVSWERSTTPDIT |
| (7Fn3) | GYRITTTPTNGQQGNSLEEVVHADQSSCTFDN |
| | LSPGLEYNVSVYTVKDDKESVPISDTIIP |
| | (SEQ ID NO: 42) |
| Pronectins | AVPPPTDLRFTNIGPDTMRVTWAPPPSIDLTN |
| (8Fn3) | FLVRYSPVKNEEDVAELSISPSDNAVVLTNLL |
| | PGTEYVVSVSSVYEQHESTPLRGRQKT |
| | (SEQ ID NO: 44) |
| Pronectins | GLDSPTGIDFSDITANSFTVHWIAPRATITGY |
| (9Fn3) | RIRHHPEHFSGRPREDRVPHSRNSITLTNLTP |
| | GTEYVVSIVALNGREESPLLIGQQST |
| | (SEQ ID NO: 46) |
| Pronectins | VSDVPRDLVVAATPTSLLISWDAPAVTVRYYR |
| (10Fn3) | ITYGETGGNSPVQEFTVPGSKSTATISGLKPG |
| | VDYTITVYAVTGRGDSPASSKPISINYRT |
| | (SEQ ID NO: 48) |
| Pronectins | EIDKPSQMQVTDVQDNSISVKWLPSSSPVTGY |
| (11Fn3) | RVTTTPKNGPGPTKTKTAGPDQTEMTIEGLQP |
| | TVEYVVSVYAQNPSGESQPLVQTAVT |
| | (SEQ ID NO: 50) |
| Pronectins | NIDRPKGLAFTDVDVDSIKIAWESPQGQVSRY |
| (12Fn3) | RVTYSSPEDGIHELFPAPDGEEDTAELQGLRP |
| | GSEYTVSVVALHDDMESQPLIGTQST |
| | (SEQ ID NO: 52) |
| Pronectins | AIPAPTDLKFTQVTPTSLSAQWTPPNVQLTGY |
| (13Fn3) | RVRVTPKEKTGPMKEINLAPDSSSVVVSGLMV |
| | ATKYEVSVYALKDTLTSRPAQGVVTTLE |
| | (SEQ ID NO: 54) |
| Pronectins | NVSPPRRARVTDATETTITISWRTKTETITGF |
| (14Fn3) | QVDAVPANGQTPIQRTIKPDVRSYTITGLQPG |
| | TDYKIYLYTLNDNARSSVVIDAST |
| | (SEQ ID NO: 56) |
| Pronectins | AIDAPSNLRFLATTPNSLLVSWQPPRARITGY |
| (15Fn3) | IIKYEKPGSPPREVVPRPRPGVTEATITGLEP |
| | GTEYTIYVIALKNNQKSEPLIGRKKT |
| | (SEQ ID NO: 58) |
| Pronectins | PGLNPNASTGQEALSQTTISWAPFQDTSEYII |
| (16Fn3) | SCHPVGTDEEPLQFRVPGTSTSATLTGLTRGA |
| | TYNIIVEALKDQQRHKVREEVVTV |
| | (SEQ ID NO: 60) |
| Adhiron | ATGVRAVPGNENSLEIEELARFAVDEHNKKEN |
| | ALLEFVRVVKAKEQVVAGTMYYLTLEAKDGGK |
| | KKLYEAKVWVKPWENFKELQEFKPVGDA |
| | (SEQ ID NO: 62) |
| Affibodies | VDNKFNKEQQNAFYEILHLPNLNEEQRNAFIQ |
| | SLKDDPSQSANLLAEAKKLNDAQAPK |
| | (SEQ ID NO: 64) |
| Affilins | GKITFYEDRAFQGRSYECTTDCPNLQPYFSRC |
| (γ-B- | NSIRVESGCWMIYERPNYQGHQYFLRRGEYPD |
| Crystallin) | YQQWMGLSDSIRSCCLIPPHSGAYRMKIYDRD |
| | ELRGQMSELTDDCISVQDRFHLTEIHSLNVLE |
| | GSWILYEMPNYRGRQYLLRPGEYRRFLDWGAP |
| | NAKVGSLRRVMDLY (SEQ ID NO: 66) |
| Affimers | MIPRGLSEAKPATPEIQEIVDKVKPQLEEKTN |
| | ETYGKLEAVQYKTQVLASTNYYIKVRAGDNKY |
| | MHLKVFNGPPGQNADRVLTGYQVDKNKDDELT |
| | GF (SEQ ID NO: 68) |
| Anticalin | IASDEEIQDVSGTWYLKAMTVDREFPEMNLES |
| (lipocalin Lcn1) | VTPMTLTTLEGGNLEAKVTMLISGRCQEVKAV |
| | LEKTDEPGKYTADGGKHVAYIIRSHVKDHYIF |
| | YSEGELHGKPVRGVKLVGRDPKNNLEALLDFE |
| | KAAGARGLSTESILIPRQSETCSPGS |
| | (SEQ ID NO: 70) |
| Anticalins | QDSTSDLIPAPPLSKVPLQQNFQDNQFQGKWY |
| (lipocalin Lcn2) | VVGLAGNAILREDKDPQKMYATIYELKEDKSY |
| | NVTSVLFRKKKCDYWIRTFVPGCQPGEFTLGN |
| | IKSYPGLTSYLVRVVSTNYNQHAMVFFKKVSQ |
| | NREYFKITLYGRTKELTSELKENFIRFSKSLG |
| | LPENHIVFPVPIDQCIDG |
| | (SEQ ID NO: 72) |
| Avimers (C426) | CESGEFQCHSTGRCIPQEWVCDGDNDCEDSSD |
| | EAPDLCASAEPTCPSGEFQCRSTNRCIPETWL |
| | CDGDNDCEDGSDEESCTPPT |
| | (SEQ ID NO: 74) |
| targeting c-MET | LPAPKNLVVSEVTEDSARLSWTAPDAAFDSFL |
| Centyrins (Fn3 | IGYGESEKVGEAIVLTVPGSERSYDLTGLKPG |
| domain of | TEYTVSIYGVKGGHRSNPLSAIFTT |
| Tenascin) | (SEQ ID NO: 76) |
| Cysknots/Knottin | CSPSGAICSGFGPPEQCCSAGCVLNRRARSWR |
| (SOTI Var.1) | CQ (SEQ ID NO: 109) |
| cleavage by AEP- | |
| likeligase in | |
| acidic is | |
| required | |
| Cysknots/Knottin | CSPSGAICSGFGPPEQCCSAGACVPHPILRIF |
| (SOTI-III) | VCQ (SEQ ID NO: 110) |
| Kalata B1 | GLPVCGETCVGGTCNTPGCTCSWPVCTRN |
| | (SEQ ID NO: 111) |
| Kalata B2 | GLPVCGETCFGGTCNTPGCSCTWPICTRD |
| | (SEQ ID NO: 112) |
| MCoTI-I | GGVCPKILQRCRRDSDCPGACICRGNGYCGSG |
| | SD (SEQ ID NO: 113) |
| MCoTI-II | GGVCPKILKKCRRDSDCPGACICRGNGYCGSG |
| | SD (SEQ ID NO: 114) |
| Kunitz | VREVCSEQAETGPCRAMISRWYFDVTEGKCAP |
| domain/BPTI | FFYGGCCGGNRNNFDTEEYCMAVCG |
| | (SEQ ID NO: 78) |
| Obodies | EIMDAAEDYAKERYGISSMIQSQEKPDRVLVR |
| (human AspRS) | VRDLTIQKADEVVWVRARVHTSRAKGKQCFLV |
| | LRQQQFNVQALVAVGDHASKQMVKFAANINKE |
| | SIVDVEGVVRKVNQKIGSCTQQDVELHVQKIY |
| | VISLAEPRLPLQLDDAVRPEAEGEEEGRATVN |
| | QDTRLDNRVIDL (SEQ ID NO: 80) |
| Tn3A | AIEVKDVTDTTALITWSDEFGHDYDGCELTYG |
| | IKDVPGDRTTIDLWWHSAWYSIGNLKPDTEDV |
| | SLICYTDQEAGNPAKETFTTGLVPR |
| | (SEQ ID NO: 82) |
| Tn3B | AIEVEDVTDTTALITWTNRSSYSNLHGCELAY |
| | GIKDVPGDRTTIDLNQPYVHYSIGNLKPDTEY |
| | EVSLICLTTDGTYNNPAKETFTTGLVPR |
| | (SEQ ID NO: 84) |
| Hckomers | TLFVALYDYEARTEDELSFHKGEKFQILNSSE |
| | GDWWEARDSLTTGETGYIPSNYVAPVD |
| | (SEQ ID NO: 86) |
| NPHP1 | EEYIAVGDFDTAQQVGDLTFKKGEILLVIEKK |
| | PDGWWIAKDAKGNEGLVPRTYLEPYS |
| | (SEQ ID NO: 88) |
| Tec | EIVVAMYDFQAAEGHDLRLERQEYLILEKNDV |
| | HWWRARDKYGNEGYIPSNYVTGKK |
| | (SEQ ID NO: 90) |
| Hck | IIVVALYDYEAIHHEDLSFQKGDQMVVLEESG |
| | EWWKARSLATRKEGYIPSNYVARVD |
| | (SEQ ID NO: 92) |
| Amph | YKVETLHDFEAANSDELTLQRGDVVLVVPSDS |
| | EADQDAGWLVGVKESDWLQYRDLATYKGLFPE |
| | NFTRRLD (SEQ ID NO: 94) |
| RIMBP#3 | KIMIAALDYDPGDGQMGGQGKGRLALRAGDVV |
| | MVYGPMDDQGFYYGELGGHRGLVPAHLLDHMS |
| | (SEQ ID NO: 96) |
| IRIKS | QKVKTIFPHTAGSNKTLLSFAQGDVITLLIPE |
| | EKDGWLYGEHDVSKARGWFPSSYTKLLE |
| | (SEQ ID NO: 98) |
| SNX33 | LKGRALYDFHSENKEEISIQQDEDLVIFSETS |
| | LDGWLQGQNSRGETGLFPASYVEIVR |
| | (SEQ ID NO: 100) |
| Eps8L1 | KWVLCNYDFQARNSSELSVKQRDVLEVLDDSR |
| | KWWKVRDPAGQEGYVPYNILTPYP |
| | (SEQ ID NO: 102) |
| FISH#5 | DVYVSIADYEGDEETAGFQEGVSMEVLERNPN |
| | GWWYCQILDGVKPFKGWVPSNYLEKKN |
| | (SEQ ID NO: 104) |
| CMS#1 | VDYIVEYDYDAVHDDELTIRVGEIIRNVKKLQ |
| | EEGWLEGELNGRRGMFPDNFVKEIK |
| | (SEQ ID NO: 106) |
| OSTF1 | KVFRALYTFEPRTPDELYFEEGDIIYITDMSD |
| | TNWWKGTSKGRTGLIPSNYVAEQA |
| | (SEQ ID NO: 108) |

TABLE 2
Nucleic acid sequence of non-Ig
scaffold proteins that can function as
antigen-binding proteins:

| Scaffold | Nucleic Acid Sequence (SEQ ID NO:) |
|---|---|
| Adnectins | GTGAGCGACGTGCCCAGAAAGCTGGAGGTGGTGGC |
| (10Fn3) | CGCCACCCCCACCAGCCTGCTGATCAGCTGGGACG |
| | CCCCCGCCGTGACCGTGAGATACTACAGAATCACCT |
| | ACGGCGAGACCGGCGGCAACAGCCCCGTGCAGGAG |
| | TTCACCGTGCCCGGCAGCAAGAGCACCGCCACCAT |
| | CAGCGGCCTGAAGCCCGGCGTGGACTACACCATCA |
| | CCGTGTACGCCGTGACCGGCAGAGGCGACAGCCCC |
| | GCCAGCAGCAAGCCCATCAGCAACTACAGAACCGC |
| | CCTGGAG (SEQ ID NO: 23) |
| Adnectin 1 | GTGAGCGACGTGCCCAGAAAGCTGGAGGTGGTGGC |
| | CGCCACCCCCACCAGCCTGCTGATCAGCTGGGACA |
| | GCGGCAGAGGCAGCTACAGATACTACAGAATCACC |
| | TACGGCGAGACCGGCGGCAACAGCCCCGTGCAGGA |
| | GTTCACCGTGCCCGGCCCCGTGCACACCGCCACCAT |
| | CAGCGGCCTGAAGCCCGGCGTGGACTACACCATCA |
| | CCGTGTACGCCGTGACCGACCACAAGCCCCACGCC |
| | GACGGCCCCCACACCTACCACGAGAGCCCCATCAG |
| | CAACTACAGAACCGCCCTGGAG (SEQ ID |
| | NO: 25) |
| Adnectin 2 | GTGAGCGACGTGCCCAGAAAGCTGGAGGTGGTGGC |
| | CGCCACCCCCACCAGCCTGCTGATCAGCTGGGAGC |
| | ACGACTACCCCTACAGAAGATACTACAGAATCACC |
| | TACGGCGAGACCGGCGGCAACAGCCCCGTGCAGGA |
| | GTTCACCGTGCCCAAGGACGTGGACACCGCCACCA |
| | TCAGCGGCCTGAAGCCCGGCGTGGACTACACCATC |
| | ACCGTGTACGCCGTGACCAGCAGCTACAAGTACGA |
| | CATGCAGTACAGCCCCATCAGCAACTACAGAACCG |
| | CCCTGGAG (SEQ ID NO: 27) |
| Pronectins | AGCGGCCCCGTGGAGGTGTTCATCACCGAGACCCC |
| (1Fn3) | CAGCCAGCCCAACAGCCACCCCATCCAGTGGAACG |
| | CCCCCCAGCCCAGCCACATCAGCAAGTACATCCTG |
| | AGATGGAGACCCAAGAACAGCGTGGGCAGATGGA |
| | AGGAGGCCACCATCCCCGGCCACCTGAACAGCTAC |
| | ACCATCAAGGGCCTGAAGCCCGGCGTGGTGTACGA |
| | GGGCCAGCTGATCAGCATCCAGCAGTACGGCCACC |
| | AGGAGGTGACCAGATTCGACTTCACCACCACC (SEQ |
| | ID NO: 29) |
| Pronectins | AGCCCCCTGGTGGCCACCAGCGAGAGCGTGACCGA |
| (2Fn3) | GATCACCGCCAGCAGCTTCGTGGTGAGCTGGGTGA |
| | GCGCCAGCGACACCGTGAGCGGCTTCAGAGTGGAG |
| | TACGAGCTGAGCGAGGAGGGCGACGAGCCCCAGTA |
| | CCTGGACCTGCCCAGCACCGCCACCAGCGTGAACA |
| | TCCCCGACCTGCTGCCCGGCAGAAAGTACATCGTG |
| | AACGTGTACCAGAGCGAGGACGGCGAGCAGAGCCT |
| | GATCCTGAGCACCAGCCAGACCACC (SEQ ID |
| | NO: 31) |
| Pronectins | GCCCCCGACGCCCCCCCCGACCCCACCGTGGACCA |
| (3Fn3) | GGTGGACGACACCAGCATCGTGGTGAGATGGAGCA |
| | GACCCCAGGCCCCCATCACCGGCTACAGAATCGTG |
| | TACAGCCCCAGCGTGGAGGGCAGCAGCACCGAGCT |
| | GAACCTGCCCGAGACCGCCAACAGCGTGACCCTGA |
| | GCGACCTGCAGCCCGGCGTGCAGTACAACATCACC |
| | ATCTACGCCGTGGAGGAGAACCAGGAGAGCACCCC |
| | CGTGGTGATCCAGCAGGAGACCACCGGCACCCCCA |
| | GA (SEQ ID NO: 33) |
| Pronectins | ACCGTGCCCAGCCCCAGAGACCTGCAGTTCGTGGA |
| (4Fn3) | GGTGACCGACGTGAAGGTGACCATCATGTGGACCC |
| | CCCCCGAGAGCGCCGTGACCGGCTACAGAGTGGAC |
| | GTGATCCCCGTGAACCTGCCCGGCGAGCACGGCCA |
| | GAGACTGCCCATCAGCAGAAACACCTTCGCCGAGG |
| | TGACCGGCCTGAGCCCCGGCGTGACCTACTACTTCA |
| | AGGTGTTCGCCGTGAGCCACGGCAGAGAGAGCAAG |
| | CCCCTGACCGCCCAGCAGACCACC (SEQ ID |
| | NO: 35) |
| Pronectins | AAGCTGGACGCCCCCACCAACCTGCAGTTCGTGAA |
| (5Fn3) | CGAGACCGACAGCACCGTGCTGGTGAGATGGACCC |
| | CCCCCAGAGCCCAGATCACCGGCTACAGACTGACC |
| | GTGGGCCTGACCAGAAGAGGCCAGCCCAGACAGTA |
| | CAACGTGGGCCCCAGCGTGAGCAAGTACCCCCTGA |
| | GAAACCTGCAGCCCGCCAGCGAGTACACCGTGAGC |
| | CTGGTGGCCATCAAGGGCAACCAGGAGAGCCCCAA |
| | GGCCACCGGCGTGTTCACCACCCTG (SEQ ID |
| | NO: 37) |
| Pronectins | CAGCCCGGCAGCAGCATCCCCCCCTACAACACCGA |
| (6Fn3) | GGTGACCGAGACCACCATCGTGATCACCTGGACCC |
| | CCGCCCCCAGACTGGGCTTCAAGCTGGGCGTGAGA |
| | CCCAGCCAGGGCGGCGAGGCCCCCAGAGAGGTGAC |
| | CAGCGACAGCGGCAGCGTGGTGAGCGGCCTGACCC |
| | CCGGCGTGGAGTACGTGTACACCATCCAGGTGCTG |
| | AGAGACGGCCAGGAGAGAGACGCCCCCATCGTGAA |
| | CAAGGTGGTGACC (SEQ ID NO: 39) |
| Pronectins | CCCCTGAGCCCCCCCACCAACCTGCACCTGGAGGCC |
| (7Fn3) | AACCCCGACACCGGCGTGCTGACCGTGAGCTGGGA |
| | GAGAAGCACCACCCCCGACATCACCGGCTACAGAA |
| | TCACCACCACCCCCACCAACGGCCAGCAGGGCAAC |
| | AGCCTGGAGGAGGTGGTGCACGCCGACCAGAGCAG |
| | CTGCACCTTCGACAACCTGAGCCCCGGCCTGGAGTA |
| | CAACGTGAGCGTGTACACCGTGAAGGACGACAAGG |
| | AGAGCGTGCCCATCAGCGACACCATCATCCCCTGA |
| | (SEQ ID NO: 41) |
| Pronectins | GCCGTGCCCCCCCCCACCGACCTGAGATTCACCAAC |
| (8Fn3) | ATCGGCCCCGACACCATGAGAGTGACCTGGGCCCC |
| | CCCCCCCAGCATCGACCTGACCAACTTCCTGGTGAG |
| | ATACAGCCCCGTGAAGAACGAGGAGGACGTGGCCG |
| | AGCTGAGCATCAGCCCCAGCGACAACGCCGTGGTG |
| | CTGACCAACCTGCTGCCCGGCACCGAGTACGTGGT |
| | GAGCGTGAGCAGCGTGTACGAGCAGCACGAGAGCA |
| | CCCCCCTGAGAGGCAGACAGAAGACCTGA (SEQ ID |
| | NO: 43) |
| Pronectins | GGCCTGGACAGCCCCACCGGCATCGACTTCAGCGA |
| (9Fn3) | CATCACCGCCAACAGCTTCACCGTGCACTGGATCGC |
| | CCCCAGAGCCACCATCACCGGCTACAGAATCAGAC |
| | ACCACCCCGAGCACTTCAGCGGCAGACCCAGAGAG |
| | GACAGAGTGCCCCACAGCAGAAACAGCATCACCCT |
| | GACCAACCTGACCCCCGGCACCGAGTACGTGGTGA |
| | GCATCGTGGCCCTGAACGGCAGAGAGGAGAGCCCC |
| | CTGCTGATCGGCCAGCAGAGCACCTGA (SEQ ID |
| | NO: 45) |
| Pronectins | GTGAGCGACGTGCCCAGAGACCTGGTGGTGGCCGC |
| (10Fn3) | CACCCCCACCAGCCTGCTGATCAGCTGGGACGCCCC |
| | CGCCGTGACCGTGAGATACTACAGAATCACCTACG |
| | GCGAGACCGGCGGCAACAGCCCCGTGCAGGAGTTC |
| | ACCGTGCCCGGCAGCAAGAGCACCGCCACCATCAG |
| | CGGCCTGAAGCCCGGCGTGGACTACACCATCACCG |
| | TGTACGCCGTGACCGGCAGAGGCGACAGCCCCGCC |
| | AGCAGCAAGCCCATCAGCATCAACTACAGAACC |
| | (SEQ ID NO: 47) |
| Pronectins | GAGATCGACAAGCCCAGCCAGATGCAGGTGACCGA |
| (11Fn3) | CGTGCAGGACAACAGCATCAGCGTGAAGTGGCTGC |
| | CCAGCAGCAGCCCCGTGACCGGCTACAGAGTGACC |
| | ACCACCCCCAAGAACGGCCCCGGCCCCACCAAGAC |
| | CAAGACCGCCGGCCCCGACCAGACCGAGATGACCA |
| | TCGAGGGCCTGCAGCCCACCGTGGAGTACGTGGTG |
| | AGCGTGTACGCCCAGAACCCCAGCGGCGAGAGCCA |
| | GCCCCTGGTGCAGACCGCCGTGACC (SEQ ID |
| | NO: 49) |
| Pronectins | AACATCGACAGACCCAAGGGCCTGGCCTTCACCGA |
| (12Fn3) | CGTGGACGTGGACAGCATCAAGATCGCCTGGGAGA |
| | GCCCCCAGGGCCAGGTGAGCAGATACAGAGTGACC |
| | TACAGCAGCCCCGAGGACGGCATCCACGAGCTGTT |
| | CCCCGCCCCCGACGGCGAGGAGGACACCGCCGAGC |
| | TGCAGGGCCTGAGACCCGGCAGCGAGTACACCGTG |
| | AGCGTGGTGGCCCTGCACGACGACATGGAGAGCCA |
| | GCCCCTGATCGGCACCCAGAGCACCTGA (SEQ ID |
| | NO: 51) |
| Pronectins | GCCATCCCCGCCCCCACCGACCTGAAGTTCACCCAG |
| (13Fn3) | GTGACCCCCACCAGCCTGAGCGCCCAGTGGACCCC |
| | CCCCAACGTGCAGCTGACCGGCTACAGAGTGAGAG |
| | TGACCCCCAAGGAGAAGACCGGCCCCATGAAGGAG |
| | ATCAACCTGGCCCCCGACAGCAGCAGCGTGGTGGT |
| | GAGCGGCCTGATGGTGGCCACCAAGTACGAGGTGA |
| | GCGTGTACGCCCTGAAGGACACCCTGACCAGCAGA |
| | CCCGCCCAGGGCGTGGTGACCACCCTGGAG (SEQ |
| | ID NO: 53) |
| Pronectins | AACGTGAGCCCCCCCAGAAGAGCCAGAGTGACCGA |
| (14Fn3) | CGCCACCGAGACCACCATCACCATCAGCTGGAGAA |
| | CCAAGACCGAGACCATCACCGGCTTCCAGGTGGAC |
| | GCCGTGCCCGCCAACGGCCAGACCCCCATCCAGAG |
| | AACCATCAAGCCCGACGTGAGAAGCTACACCATCA |
| | CCGGCCTGCAGCCCGGCACCGACTACAAGATCTAC |
| | CTGTACACCCTGAACGACAACGCCAGAAGCAGCGT |
| | GGTGATCGACGCCAGCACC (SEQ ID NO: 55) |
| Pronectins | GCCATCGACGCCCCCAGCAACCTGAGATTCCTGGCC |
| (15Fn3) | ACCACCCCCAACAGCCTGCTGGTGAGCTGGCAGCC |
| | CCCCAGAGCCAGAATCACCGGCTACATCATCAAGT |
| | ACGAGAAGCCCGGCAGCCCCCCCAGAGAGGTGGTG |
| | CCCAGACCCAGACCCGGCGTGACCGAGGCCACCAT |
| | CACCGGCCTGGAGCCCGGCACCGAGTACACCATCT |
| | ACGTGATCGCCCTGAAGAACAACCAGAAGAGCGAG |
| | CCCCTGATCGGCAGAAAGAAGACC (SEQ ID |
| | NO: 57) |
| Pronectins | CCCGGCCTGAACCCCAACGCCAGCACCGGCCAGGA |
| (16Fn3) | GGCCCTGAGCCAGACCACCATCAGCTGGGCCCCCTT |
| | CCAGGACACCAGCGAGTACATCATCAGCTGCCACC |
| | CCGTGGGCACCGACGAGGAGCCCCTGCAGTTCAGA |
| | GTGCCCGGCACCAGCACCAGCGCCACCCTGACCGG |
| | CCTGACCAGAGGCGCCACCTACAACATCATCGTGG |
| | AGGCCCTGAAGGACCAGCAGAGACACAAGGTGAG |
| | AGAGGAGGTGGTGACCGTG (SEQ ID NO: 59) |
| Adhiron | GCCACCGGCGTGAGAGCCGTGCCCGGCAACGAGAA |
| | CAGCCTGGAGATCGAGGAGCTGGCCAGATTCGCCG |
| | TGGACGAGCACAACAAGAAGGAGAACGCCCTGCTG |
| | GAGTTCGTGAGAGTGGTGAAGGCCAAGGAGCAGGT |
| | GGTGGCCGGCACCATGTACTACCTGACCCTGGAGG |
| | CCAAGGACGGCGGCAAGAAGAAGCTGTACGAGGCC |
| | AAGGTGTGGGTGAAGCCCTGGGAGAACTTCAAGGA |
| | GCTGCAGGAGTTCAAGCCCGTGGGCGACGCC (SEQ |
| | ID NO: 61) |
| Affibodies | GTGGACAACAAGTTCAACAAGGAGCAGCAGAACGC |
| | CTTCTACGAGATCCTGCACCTGCCCAACCTGAACGA |
| | GGAGCAGAGAAACGCCTTCATCCAGAGCCTGAAGG |
| | ACGACCCCAGCCAGAGCGCCAACCTGCTGGCCGAG |
| | GCCAAGAAGCTGAACGACGCCCAGGCCCCCAAGTG |
| | A (SEQ ID NO: 63) |
| Affilins | GGCAAGATCACCTTCTACGAGGACAGAGCCTTCCA |
| (γ-B | GGGCAGAAGCTACGAGTGCACCACCGACTGCCCCA |
| Crystallin) | ACCTGCAGCCCTACTTCAGCAGATGCAACAGCATC |
| | AGAGTGGAGAGCGGCTGCTGGATGATCTACGAGAG |
| | ACCCAACTACCAGGGCCACCAGTACTTCCTGAGAA |
| | GAGGCGAGTACCCCGACTACCAGCAGTGGATGGGC |
| | CTGAGCGACAGCATCAGAAGCTGCTGCCTGATCCC |
| | CCCCCACAGCGGCGCCTACAGAATGAAGATCTACG |
| | ACAGAGACGAGCTGAGAGGCCAGATGAGCGAGCTG |
| | ACCGACGACTGCATCAGCGTGCAGGACAGATTCCA |
| | CCTGACCGAGATCCACAGCCTGAACGTGCTGGAGG |
| | GCAGCTGGATCCTGTACGAGATGCCCAACTACAGA |
| | GGCAGACAGTACCTGCTGAGACCCGGCGAGTACAG |
| | AAGATTCCTGGACTGGGGCGCCCCCAACGCCAAGG |
| | TGGGCAGCCTGAGAAGAGTGATGGACCTGTAC |
| | (SEQ ID NO: 65) |
| Affimers | ATGATCCCCAGAGGCCTGAGCGAGGCCAAGCCCGC |
| | CACCCCCGAGATCCAGGAGATCGTGGACAAGGTGA |
| | AGCCCCAGCTGGAGGAGAAGACCAACGAGACCTAC |
| | GGCAAGCTGGAGGCCGTGCAGTACAAGACCCAGGT |
| | GCTGGCCAGCACCAACTACTACATCAAGGTGAGAG |
| | CCGGCGACAACAAGTACATGCACCTGAAGGTGTTC |
| | AACGGCCCCCCCGGCCAGAACGCCGACAGAGTGCT |
| | GACCGGCTACCAGGTGGACAAGAACAAGGACGACG |
| | AGCTGACCGGCTTC (SEQ ID NO: 67) |
| Anticalin | ATCGCCAGCGACGAGGAGATCCAGGACGTGAGCGG |
| (lipocalin | CACCTGGTACCTGAAGGCCATGACCGTGGACAGAG |
| Lcn1) | AGTTCCCCGAGATGAACCTGGAGAGCGTGACCCCC |
| | ATGACCCTGACCACCCTGGAGGGCGGCAACCTGGA |
| | GGCCAAGGTGACCATGCTGATCAGCGGCAGATGCC |
| | AGGAGGTGAAGGCCGTGCTGGAGAAGACCGACGA |
| | GCCCGGCAAGTACACCGCCGACGGCGGCAAGCACG |
| | TGGCCTACATCATCAGAAGCCACGTGAAGGACCAC |
| | TACATCTTCTACAGCGAGGGCGAGCTGCACGGCAA |
| | GCCCGTGAGAGGCGTGAAGCTGGTGGGCAGAGACC |
| | CCAAGAACAACCTGGAGGCCCTGCTGGACTTCGAG |
| | AAGGCCGCCGGCGCCAGAGGCCTGAGCACCGAGAG |
| | CATCCTGATCCCCAGACAGAGCGAGACCTGCAGCC |
| | CCGGCAGC (SEQ ID NO: 69) |
| Anticalins | CAGGACAGCACCAGCGACCTGATCCCCGCCCCCCC |
| (lipocalin | CCTGAGCAAGGTGCCCCTGCAGCAGAACTTCCAGG |
| Lcn2) | ACAACCAGTTCCAGGGCAAGTGGTACGTGGTGGGC |
| | CTGGCCGGCAACGCCATCCTGAGAGAGGACAAGGA |
| | CCCCCAGAAGATGTACGCCACCATCTACGAGCTGA |
| | AGGAGGACAAGAGCTACAACGTGACCAGCGTGCTG |
| | TTCAGAAAGAAGAAGTGCGACTACTGGATCAGAAC |
| | CTTCGTGCCCGGCTGCCAGCCCGGCGAGTTCACCCT |
| | GGGCAACATCAAGAGCTACCCCGGCCTGACCAGCT |
| | ACCTGGTGAGAGTGGTGAGCACCAACTACAACCAG |
| | CACGCCATGGTGTTCTTCAAGAAGGTGAGCCAGAA |
| | CAGAGAGTACTTCAAGATCACCCTGTACGGCAGAA |
| | CCAAGGAGCTGACCAGCGAGCTGAAGGAGAACTTC |
| | ATCAGATTCAGCAAGAGCCTGGGCCTGCCCGAGAA |
| | CCACATCGTGTTCCCCGTGCCCATCGACCAGTGCAT |
| | CGACGGC (SEQ ID NO: 71) |
| Avimers | TGCGAGAGCGGCGAGTTCCAGTGCCACAGCACCGG |
| (C426) | CAGATGCATCCCCCAGGAGTGGGTGTGCGACGGCG |
| targeting | ACAACGACTGCGAGGACAGCAGCGACGAGGCCCCC |
| c-MET | GACCTGTGCGCCAGCGCCGAGCCCACCTGCCCCAG |
| | CGGCGAGTTCCAGTGCAGAAGCACCAACAGATGCA |
| | TCCCCGAGACCTGGCTGTGCGACGGCGACAACGAC |
| | TGCGAGGACGGCAGCGACGAGGAGAGCTGCACCCC |
| | CCCCACCTGA (SEQ ID NO: 73) |
| Centyrins | CTGCCCGCCCCCAAGAACCTGGTGGTGAGCGAGGT |
| (Fn3 domain | GACCGAGGACAGCGCCAGACTGAGCTGGACCGCCC |
| of Tenascin) | CCGACGCCGCCTTCGACAGCTTCCTGATCGGCTACG |
| | GCGAGAGCGAGAAGGTGGGCGAGGCCATCGTGCTG |
| | ACCGTGCCCGGCAGCGAGAGAAGCTACGACCTGAC |
| | CGGCCTGAAGCCCGGCACCGAGTACACCGTGAGCA |
| | TCTACGGCGTGAAGGGCGGCCACAGAAGCAACCCC |
| | CTGAGCGCCATCTTCACCACC (SEQ ID NO: 75) |
| Kunitz | GTGAGAGAGGTGTGCAGCGAGCAGGCCGAGACCGG |
| domain/BPTI | CCCCTGCAGAGCCATGATCAGCAGATGGTACTTCG |
| | ACGTGACCGAGGGCAAGTGCGCCCCCTTCTTCTACG |
| | GCGGCTGCTGCGGCGGCAACAGAAACAACTTCGAC |
| | ACCGAGGAGTACTGCATGGCCGTGTGCGGC |
| | (SEQ ID NO: 77) |
| Obodies | GAGATCATGGACGCCGCCGAGGACTACGCCAAGGA |
| (human | GAGATACGGCATCAGCAGCATGATCCAGAGCCAGG |
| AspRS) | AGAAGCCCGACAGAGTGCTGGTGAGAGTGAGAGAC |
| | CTGACCATCCAGAAGGCCGACGAGGTGGTGTGGGT |
| | GAGAGCCAGAGTGCACACCAGCAGAGCCAAGGGC |
| | AAGCAGTGCTTCCTGGTGCTGAGACAGCAGCAGTT |
| | CAACGTGCAGGCCCTGGTGGCCGTGGGCGACCACG |
| | CCAGCAAGCAGATGGTGAAGTTCGCCGCCAACATC |
| | AACAAGGAGAGCATCGTGGACGTGGAGGGCGTGGT |
| | GAGAAAGGTGAACCAGAAGATCGGCAGCTGCACCC |
| | AGCAGGACGTGGAGCTGCACGTGCAGAAGATCTAC |
| | GTGATCAGCCTGGCCGAGCCCAGACTGCCCCTGCA |
| | GCTGGACGACGCCGTGAGACCCGAGGCCGAGGGCG |
| | AGGAGGAGGGCAGAGCCACCGTGAACCAGGACAC |
| | CAGACTGGACAACAGAGTGATCGACCTG (SEQ ID |
| | NO: 79) |
| Tn3A | GCCATCGAGGTGAAGGACGTGACCGACACCACCGC |
| | CCTGATCACCTGGAGCGACGAGTTCGGCCACGACT |
| | ACGACGGCTGCGAGCTGACCTACGGCATCAAGGAC |
| | GTGCCCGGCGACAGAACCACCATCGACCTGTGGTG |
| | GCACAGCGCCTGGTACAGCATCGGCAACCTGAAGC |
| | CCGACACCGAGGACGTGAGCCTGATCTGCTACACC |
| | GACCAGGAGGCCGGCAACCCCGCCAAGGAGACCTT |
| | CACCACCGGCCTGGTGCCCAGA (SEQ ID |
| | NO: 81) |
| Tn3B | GCCATCGAGGTGGAGGACGTGACCGACACCACCGC |
| | CCTGATCACCTGGACCAACAGAAGCAGCTACAGCA |
| | ACCTGCACGGCTGCGAGCTGGCCTACGGCATCAAG |
| | GACGTGCCCGGCGACAGAACCACCATCGACCTGAA |
| | CCAGCCCTACGTGCACTACAGCATCGGCAACCTGA |
| | AGCCCGACACCGAGTACGAGGTGAGCCTGATCTGC |
| | CTGACCACCGACGGCACCTACAACAACCCCGCCAA |
| | GGAGACCTTCACCACCGGCCTGGTGCCCAGA |
| | (SEQ ID NO: 83) |
| Hckomers | ACCCTGTTCGTGGCCCTGTACGACTACGAGGCCAGA |
| | ACCGAGGACGAGCTGAGCTTCCACAAGGGCGAGAA |
| | GTTCCAGATCCTGAACAGCAGCGAGGGCGACTGGT |
| | GGGAGGCCAGAGACAGCCTGACCACCGGCGAGACC |
| | GGCTACATCCCCAGCAACTACGTGGCCCCCGTGGA |
| | C (SEQ ID NO: 85) |
| NPHP1 | GAGGAGTACATCGCCGTGGGCGACTTCGACACCGC |
| | CCAGCAGGTGGGCGACCTGACCTTCAAGAAGGGCG |
| | AGATCCTGCTGGTGATCGAGAAGAAGCCCGACGGC |
| | TGGTGGATCGCCAAGGACGCCAAGGGCAACGAGGG |
| | CCTGGTGCCCAGAACCTACCTGGAGCCCTACAGC |
| | (SEQ ID NO: 87) |
| Tec | GAGATCGTGGTGGCCATGTACGACTTCCAGGCCGC |
| | CGAGGGCCACGACCTGAGACTGGAGAGACAGGAGT |
| | ACCTGATCCTGGAGAAGAACGACGTGCACTGGTGG |
| | AGAGCCAGAGACAAGTACGGCAACGAGGGCTACAT |
| | CCCCAGCAACTACGTGACCGGCAAGAAGTGA (SEQ |
| | ID NO: 89) |
| Hck | ATCATCGTGGTGGCCCTGTACGACTACGAGGCCATC |
| | CACCACGAGGACCTGAGCTTCCAGAAGGGCGACCA |
| | GATGGTGGTGCTGGAGGAGAGCGGCGAGTGGTGGA |
| | AGGCCAGAAGCCTGGCCACCAGAAAGGAGGGCTAC |
| | ATCCCCAGCAACTACGTGGCCAGAGTGGAC (SEQ ID |
| | NO: 91) |
| Amph | TACAAGGTGGAGACCCTGCACGACTTCGAGGCCGC |
| | CAACAGCGACGAGCTGACCCTGCAGAGAGGCGACG |
| | TGGTGCTGGTGGTGCCCAGCGACAGCGAGGCCGAC |
| | CAGGACGCCGGCTGGCTGGTGGGCGTGAAGGAGAG |
| | CGACTGGCTGCAGTACAGAGACCTGGCCACCTACA |
| | AGGGCCTGTTCCCCGAGAACTTCACCAGAAGACTG |
| | GAC (SEQ ID NO: 93) |
| RIMBP#3 | AAGATCATGATCGCCGCCCTGGACTACGACCCCGG |
| | CGACGGCCAGATGGGCGGCCAGGGCAAGGGCAGA |
| | CTGGCCCTGAGAGCCGGCGACGTGGTGATGGTGTA |
| | CGGCCCCATGGACGACCAGGGCTTCTACTACGGCG |
| | AGCTGGGCGGCCACAGAGGCCTGGTGCCCGCCCAC |
| | CTGCTGGACCACATGAGC (SEQ ID NO: 95) |
| IRIKS | CAGAAGGTGAAGACCATCTTCCCCCACACCGCCGG |
| | CAGCAACAAGACCCTGCTGAGCTTCGCCCAGGGCG |
| | ACGTGATCACCCTGCTGATCCCCGAGGAGAAGGAC |
| | GGCTGGCTGTACGGCGAGCACGACGTGAGCAAGGC |
| | CAGAGGCTGGTTCCCCAGCAGCTACACCAAGCTGC |
| | TGGAG (SEQ ID NO: 97) |
| SNX33 | CTGAAGGGCAGAGCCCTGTACGACTTCCACAGCGA |
| | GAACAAGGAGGAGATCAGCATCCAGCAGGACGAG |
| | GACCTGGTGATCTTCAGCGAGACCAGCCTGGACGG |
| | CTGGCTGCAGGGCCAGAACAGCAGAGGCGAGACCG |
| | GCCTGTTCCCCGCCAGCTACGTGGAGATCGTGAGA |
| | (SEQ ID NO: 99) |
| Eps8L1 | AAGTGGGTGCTGTGCAACTACGACTTCCAGGCCAG |
| | AAACAGCAGCGAGCTGAGCGTGAAGCAGAGAGAC |
| | GTGCTGGAGGTGCTGGACGACAGCAGAAAGTGGTG |
| | GAAGGTGAGAGACCCCGCCGGCCAGGAGGGCTACG |
| | TGCCCTACAACATCCTGACCCCCTACCCC (SEQ ID |
| | NO: 101) |
| FISH#5 | GACGTGTACGTGAGCATCGCCGACTACGAGGGCGA |
| | CGAGGAGACCGCCGGCTTCCAGGAGGGCGTGAGCA |
| | TGGAGGTGCTGGAGAGAAACCCCAACGGCTGGTGG |
| | TACTGCCAGATCCTGGACGGCGTGAAGCCCTTCAA |
| | GGGCTGGGTGCCCAGCAACTACCTGGAGAAGAAGA |
| | AC (SEQ ID NO: 103) |
| CMS#1 | GTGGACTACATCGTGGAGTACGACTACGACGCCGT |
| | GCACGACGACGAGCTGACCATCAGAGTGGGCGAGA |
| | TCATCAGAAACGTGAAGAAGCTGCAGGAGGAGGGC |
| | TGGCTGGAGGGCGAGCTGAACGGCAGAAGAGGCAT |
| | GTTCCCCGACAACTTCGTGAAGGAGATCAAG |
| | (SEQ ID NO: 105) |
| OSTF1 | AAGGTGTTCAGAGCCCTGTACACCTTCGAGCCCAG |
| | AACCCCCGACGAGCTGTACTTCGAGGAGGGCGACA |
| | TCATCTACATCACCGACATGAGCGACACCAACTGGT |
| | GGAAGGGCACCAGCAAGGGCAGAACCGGCCTGATC |
| | CCCAGCAACTACGTGGCCGAGCAGGCC (SEQ ID |
| | NO: 107) |

The disclosure provides viral vectors the contain a heterologous polynucleotide encoding, for example a CAR with an antigen binding domain (e.g., antibody or antibody fragment; or non-antibody binding domain such as a non-immunoglobulin (Ig) scaffold protein, or combinations of coding sequences etc.), that can be delivered to a cell or directly to a subject. The viral vector can be an adenoviral vector, a measles vector, a herpes vector, a retroviral vector (including Alpha-, Beta-, Gamma-, Delta-retroviral vector, Spumavirus vector such as Simian Foamy Virus (SFV) or Human Foamy Virus (HFV), or lentiviral vector), a rhabdoviral vector such as a Vesicular Stomatitis viral vector, a reovirus vector, a Seneca Valley Virus vector, a poxvirus vector (including animal pox or vaccinia derived vectors), a parvovirus vector (including an AAV vector), an alphavirus vector or other viral vector known to one skilled in the art (see also, e.g., Concepts in Genetic Medicine, ed. Boro Dropulic and Barrie Carter, Wiley, 2008, Hoboken, N.J.; The Development of Human Gene Therapy, ed. Theodore Friedmann, Cold Springs Harbor Laboratory Press, Cold springs Harbor, New York, 1999; Gene and Cell Therapy, ed. Nancy Smyth Templeton, Marcel Dekker Inc., New York, N.Y., 2000 and Gene & Cell Therapy: Therapeutic Mechanism and Strategies, 3rd. ed., ed. Nancy Smyth Templetone, CRC Press, Boca Raton, Fla., 2008; the disclosures of which are incorporated herein by reference).

As described below and above, the retroviral vectors of the disclosure can be derived from (i.e., the parental nucleotide sequence is obtained from) MLV, MoMLV, GALV, FELV, HIV and the like and are engineered to contain a sequence that encodes a CAR or CAR-like-peptide in which the conventional scFv antigen binding domain is substituted directly or through a linker by another type of antigen binding domain so that, in a cell transduced with such a vector and expressing the CAR or CAR-like protein, the zeta chain at the other end of the membrane embedded CAR-like molecule causes cell activation upon antigen binding. Here the other type of antigen binding domain can be another scFv or antibody fragemt or a non-Ig-molecule such as described here (e.g. an adenectin, a pronectin, an affimer, a hckamer, or an anti-calin).

The term “antibody,” as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be ‘humanized’, ‘chimeric’ or non-human.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′h, Fv fragments, scFv antibody fragments, disulfide-linked Fvs (sdFv), a Fd fragment consisting of the VH and CH1 domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No.: 6,703,199, which describes fibronectin polypeptide mini bodies).

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “anticancer effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition.

“Anticancer agent” refers to agents that inhibit aberrant cellular division and growth, inhibit migration of neoplastic cells, inhibit invasiveness or prevent cancer growth and metastasis. The term includes chemotherapeutic agents, biological agent (e.g., siRNA, viral vectors such as engineered MLV, adenoviruses, herpes virus that deliver cytotoxic genes), antibodies and the like.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

Non-limiting examples of target antigens include: CDS, CD19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase 1X (CA1X); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Ab1) (bcr-ab1); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGSS); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTI); Cancer/testis antigen 1 (NY-ES0-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P4501B 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLU), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GM1, PTK7, gpNMB, CDH1-CD324, DLL3, CD276/B7H3, IL11Ra, IL13Ra2, CD179b-IGLl1, TCRgamma-delta, NKG2D, CD32 (FCGR2A), Tn ag, Tim1-/HVCR1, CSF2RA (GM-CSFR-alpha), TGFbetaR2, Lews Ag, TCR-betal chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLV1-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRCSD, Claudin18.2 (CLD18A2 or CLDN18A.2)), P-glycoprotein, STEAP1, Livl, Nectin-4, Cripto, gpA33, BST1/CD157, low conductance chloride channel, and the antigen recognized by TNT antibody.

As used herein “affinity” is meant to describe a measure of binding strength. Affinity, in some instances, depends on the closeness of stereochemical fit between a binding agent and its target (e.g., between an antibody and antigen including epitopes specific for the binding domain), on the size of the area of contact between them, and on the distribution of charged and hydrophobic groups. Affinity generally refers to the “ability” of the binding agent to bind its target. There are numerous ways used in the art to measure “affinity”. For example, methods for calculating the affinity of an antibody for an antigen are known in the art, including use of binding experiments to calculate affinity. Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry. An exemplary method for determining binding affinity employs surface plasmon resonance. Surface plasmon resonance is an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.).

An “antigen binding domain” refers to a polypeptide or peptide that due to its primary, secondary or tertiary sequence and or post-translational modifications and/or charge binds to an antigen with a high degree of specificity. The antigen binding domain may be derived from different sources, for example, an antibody, a non-immunoglobulin binding protein such as an affibody, affimer, DARPin or Pronectin (Skrlec et al., Trends Biotechnol., 33:408-418, 2015), a ligand or a receptor.

“Avidity” refers to the strength of the interaction between a binding agent and its target (e.g., the strength of the interaction between an antibody and its antigen target, a receptor and its cognate and the like). The avidity can be weak or strong. Methods for calculating the affinity of an antibody for an antigen are known in the art, including use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

The term “Association constant (Ka)” is defined as the equilibrium constant of the association of a receptor and ligand.

The term “autoantigen” refers to an endogenous antigen that stimulates production of an autoimmune response, such as production of autoantibodies. Autoantigen also includes a self-antigen or antigen from a normal tissue that is the target of a cell mediated or an antibody-mediated immune response that may result in the development of an autoimmune disease. Examples of autoantigens include, but are not limited to, desmoglein 1, desmoglein 3, and fragments thereof.

As used herein “beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy.

As used herein, the term “binding domain” or “antibody molecule” refers to a protein, e.g., an immunoglobulin chain or fragment thereof, comprising at least one domain, e.g., immunoglobulin variable domain sequence that can bind to a target with affinity higher than a non-specific domain. The term encompasses antibodies and antibody fragments and also other non-immunoglobulin protein binding domains (Skrlec et al., supra). In another embodiment, an antibody molecule is a multi-specific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In another embodiment, a multi-specific antibody molecule is a bispecific antibody molecule. A bispecific antibody has specificity for two antigens. A bispecific antibody molecule is characterized by a first immunoglobulin variable domain sequence which has binding specificity for a first epitope and a second immunoglobulin variable domain sequence that has binding specificity for a second epitope.

“Cancer” and “cancerous” refer to or describe the physiological condition in mammals that is typically characterized by unregulated cell growth. Examples of cancer include, but are not limited to B-cell lymphomas (Hodgkin's lymphomas and/or non-Hodgkins lymphomas), T cell lymphomas, myeloma, myelodysplastic syndrome, skin cancer, brain tumor, breast cancer, colon cancer, rectal cancer, esophageal cancer, anal cancer, cancer of unknown primary site, endocrine cancer, testicular cancer, lung cancer, hepatocellular cancer, gastric cancer, pancreatic cancer, cervical cancer, ovarian cancer, liver cancer, bladder cancer, cancer of the urinary tract, cancer of reproductive organs thyroid cancer, renal cancer, carcinoma, melanoma, head and neck cancer, brain cancer (e.g., glioblastoma multiforme), prostate cancer, including but not limited to androgen-dependent prostate cancer and androgen-independent prostate cancer, and leukemia. Other cancer and cell proliferative disorders will be readily recognized in the art. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Chemotherapeutic agents” are compounds that are known to be of use in chemotherapy for cancer. Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above or combinations thereof

“Chimeric antigen receptors” (CARs) are artificial T cell receptors contemplated for use as a targeted therapy for cancer, using a technique called adoptive cell transfer. The essential antigen-binding, signaling, and stimulatory functions of the receptor polypeptide have been reduced by genetic recombination methods to a single polypeptide chain, generally referred to as a Chimeric Antigen Receptor (CAR) (See, e.g., Eshhar, U.S. Pat. No. 7,741,465; Eshhar, U.S. Patent Application Publication No. 2012/0093842). CARs are constructed specifically to stimulate T cell activation and proliferation in response to a specific antigen to which the CAR binds. The term “Chimeric Antigen Receptor” or alternatively a “CAR” refers to a set of polypeptides, typically two in the simplest embodiments, which when expressed in an immune effector cell, provides the cell with specificity for a target cell, typically a cancer cell, and with intracellular signal generation. In some embodiments, a CAR comprises at least an extracellular antigen binding domain, a transmembrane domain (see, e.g., SEQ ID NO:1) and a cytoplasmic signaling domain (also referred to herein as “an intracellular signaling domain”) comprising a functional signaling domain derived from a stimulatory molecule and/or costimulatory molecule (see, e.g., SEQ ID NO:5). In some aspects, the set of polypeptides are contiguous with each other. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 4-1BB (i.e., CD137; SEQ ID NO:5), CD27 and/or CD28. In one aspect the CAR comprises an optional leader sequence at the amino-terminus (N-terminus) of a CAR fusion polypeptide (see, e.g., SEQ ID NO:6). In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. Typically “CAR-T cells” are used, which refer to T-cells that have been engineered to contain a chimeric antigen receptor. Thus, T lymphocytes bearing such CARs are generally referred to as CAR-T lymphocytes.

“Codon optimization” or “controlling for species codon bias” refers to the preferred codon usage of a particular host cell. As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons.

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. Those of skill in the art will recognize that, due to the degenerate nature of the genetic code, a variety of DNA compounds differing in their nucleotide sequences can be used to encode a given polypeptide of the disclosure.

A “conservative substitution” or “conservative sequence modifications” refers to amino acid modifications that do not significantly affect or alter the binding characteristics or function of the encoded protein. For example, “conservative sequence modifications” refers to amino acid modifications that do not significantly affect or alter the binding characteristics or function of the TCR constant chain, antibody, antibody fragment, or non-immunoglobulin binding domains. Such conservative modifications include amino acid substitutions, additions and deletions. Modifications can be introduced into a TCR constant chain, antibody or antibody fragment, the non-immunoglobulin binding domain or other proteins or polypeptides of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a SIR of the disclosure can be replaced with other amino acid residues from the same side chain family and the altered SIR can be tested using the binding and/or functional assays described herein.

“Derived from” as that term is used herein, indicates a relationship between a first and a second molecule. it generally refers to structural similarity between the first molecule and a second molecule and does not connotate or include a process or source limitation on a first molecule that is derived from a second molecule. For example, in the case of an antigen binding domain that is derived from an antibody molecule, the antigen binding domain retains sufficient antibody structure such that is has the required function, namely, the ability to bind to an antigen. It does not connotate or include a limitation to a particular process of producing the antibody, e.g., it does not mean that, to provide the antigen binding domain, one must start with an antibody sequence and delete unwanted sequence, or impose mutations, to arrive at the antigen binding domain.

An “intracellular signaling domain,” as the term is used herein, refers to an intracellular signaling portion of a molecule. The intracellular signaling domain generates a signal that promotes an immune effector function of an immune cells. Examples of immune effector function include cytolytic activity and helper activity, including the secretion of cytokines.

In another embodiment, the intracellular signaling domain can comprise a primary intracellular signaling domain. Exemplary primary intracellular signaling domains include those derived from the molecules responsible for primary stimulation, or antigen dependent simulation. In another embodiment, the intracellular signaling domain can comprise a costimulatory intracellular domain. Exemplary costimulatory intracellular signaling domains include those derived from molecules responsible for costimulatory signals, or antigen independent stimulation. For example, a primary intracellular signaling domain can comprise a cytoplasmic sequence of CD3z, and a costimulatory intracellular signaling domain can comprise cytoplasmic sequence from co-receptor or costimulatory molecule, such as CD28 or 41BB.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FeR gamma (FCER1G), Fe gamma RIIa, FeR beta (Fe Epsilon R1b), CD3 gamma, CD3 delta, CD3 epsilon, CD79a, CD79b, DAP1O, and DAP12.

The term “flexible polypeptide linker” as used in refers to a peptide linker that consists of amino acids such as glycine and/or serine residues used alone or in combination, to link polypeptide chains together (e.g., variable heavy and variable light chain regions together). In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3. n=4, n=5 and n=6, n-7, n-8, n-9 and n-10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser).

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “operably linked” refers to functional linkage or association between a first component and a second component such that each component can be functional. For example, operably linked includes the association between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In the context of two polypeptides that are operably linked a first polypeptide functions in the manner it would independent of any linkage and the second polypeptide functions as it would absent a linkage between the two.

“Percent identity” in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%,81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, generally one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that can be used for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Bioi. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Bioi. 48:444-453) algorithm which has been incorporated into the GAP program in the GCG software package (available at www.gcg.com), using either a Blossom 62 matrix or a P AM250 matrix, and a gap weight of 16, 14, 12, 10, 8, 6, or 4 and a length weight of 1, 2, 3, 4, 5, or 6.

The term “polynucleotide”, “nucleic acid”, or “recombinant nucleic acid” refers to polymers of nucleotides such as deoxyribonucleic acid (DNA), and, where appropriate, ribonucleic acid (RNA). One of skill in the art will recognize that any DNA sequence provided herein can be converted to an RNA sequence by replacing ‘T’ with ‘U’.

A “protein” or “polypeptide”, which terms are used interchangeably herein, comprises one or more chains of chemical building blocks called amino acids that are linked together by chemical bonds called peptide bonds.

The term “RNV” refers to a non-replicating viral vector, and may be a retroviral vector comprising long terminal repeats, packaging signals and a cloning site (see, SEQ ID NO:12, which comprises the RNA backbone sequence of a construct of the disclosure). An RNV of the disclosure is produced from a plasmid comprising SEQ ID NO:3 transformed into a producer/packaging cell line such as an HT1080 derived packaging cell line HA-LB (Sheridan et al., Mol. Ther., 2:262-275,2000) or the similarly constructed HA-L2 packaging cell line, or by transfection with the appropriate helper genomes encoding viral protein into 293T of other efficient producer cell line. For example, an RNV can be produced by transfecting a packaging cell line comprising a gag, pol and env coding sequence with the plasmid of SEQ ID NO:3 comprising packaging signals. The method produces RNVs in which the retroviral genome is packaged in a capsid and envelope, through the use of a packaging cell. The packaging cells are provided with viral protein-coding sequences, typically in the form of two plasmids integrated into the genome of the cell, which produce all proteins necessary for production of viable retroviral particles, a DNA viral construct which codes for an RNA which will carry the desired gene, along with a packaging signal which will direct packaging of the RNA into the retroviral particles.

The term “RNVCAR” refers to a non-replicating viral vector comprising long terminal repeats, packaging signals and containing a coding sequence for a chimeric antigen receptor (CAR). The CAR comprises a binding domain that targets and selectively binds to any number of antigens (e.g., cancer antigens etc.). In some instances the disclosure uses “X”-RNVCAR, wherein Xis the name or acronym of a specific antigen (e.g., CD19-RNVCAR). The antigen can be targeted by any number of different binding domains as described herein (e.g., scFvs). The sequences for various binding domains useful in the preparations of CAR constructs are known (see, e.g., International Application Publication WO 2018/102795 at Table 5; the sequences and disclosure of which is incorporated herein by reference).

The term “RRV” refers to a replicating viral vector, and may be a retroviral vector comprising long terminal repeats, gag, pol, env, packaging signals and a cloning site. An RRV of the disclosure does not require a help cell in order to produce infectious virons. In some embodiments the RRV comprises a gammaretroviral GAG protein; a gammaretroviral POL protein; a gammaretroviral envelope; a gammaretroviral RNA polynucleotide comprising 3′ untranslated region (U3) and repeat region (R) sequences from murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus (XMRV), avian reticuloendotheliosis virus (REV), or Gibbon ape leukemia virus (GALV) at the 3′ end of the gammaretroviral polynucleotide sequence, an R and 5′ untranslated region (U5) sequence from MLV, MoMLV, FeLV, BEV, PERV, RD114, squirrel monkey retrovirus, XMRV, REV or GALV at the 5′ end of the gammaretroviral polynucleotide, a gag nucleic acid domain, a pol nucleic acid domain and an env nucleic acid domain from MLV, MoMLV, FeLV, BEV, PERV, RD114, squirrel monkey retrovirus, XMRV, REV or GALV located between the U5 and U3 regions; a cassette comprising an internal ribosome entry site (IRES), a minipromoter, or a 2A or 2A-like sequence upstream and operably linked to a heterologous polynucleotide encoding an CAR coding sequence, wherein the cassette is positioned 5′ to the U3 region and 3′ to the env nucleic acid domain; and cis-acting sequences necessary for reverse transcription, packaging and integration in a target cell.

The term “RRVCAR” refers to a replicating viral vector comprising, for example, a gammaretroviral GAG protein; a gammaretroviral POL protein; a gammaretroviral envelope; a gammaretroviral RNA polynucleotide comprising 3′ untranslated region (U3) and repeat region (R) sequences from murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus (XMRV), avian reticuloendotheliosis virus (REV), or Gibbon ape leukemia virus (GALV) at the 3′ end of the gammaretroviral polynucleotide sequence, an R and 5′ untranslated region (U5) sequence from MLV, MoMLV, FeLV, BEV, PERV, RD114, squirrel monkey retrovirus, XMRV, REV or GALV at the 5′ end of the gammaretroviral polynucleotide, a gag nucleic acid domain, a pol nucleic acid domain and an env nucleic acid domain from MLV, MoMLV, FeLV, BEV, PERV, RD114, squirrel monkey retrovirus, XMRV, REV or GALV located between the U5 and U3 regions; a cassette comprising an internal ribosome entry site (IRES), a minipromoter, or a 2A or 2A-like sequence upstream and operably linked to a heterologous polynucleotide encoding an CAR coding sequence, wherein the cassette is positioned 5′ to the U3 region and 3′ to the env nucleic acid domain; and cis-acting sequences necessary for reverse transcription, packaging and integration in a target cell. The CAR comprises a binding domain that targets and selectively binds to any number of antigens (e.g., cancer antigens etc.). In some instances the disclosure uses “X”-RRVCAR, wherein X is the name or acronym of a specific antigen (e.g., CD19-RRVCAR). The antigen can be targeted by any number of different binding domains as described herein (e.g., scFvs). The sequences for various binding domains useful in the preparations of CAR constructs are known (see, e.g., International Application Publication WO 2018/102795 at Table 5; the sequences and disclosure of which is incorporated herein by reference).

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless otherwise stated, an scFv may have the vL and vH variable regions in either order, e.g., with respect to the N-terminal and C-terminal ends of the polypeptide, the scFv may comprise vL-(linker)-vH or may comprise vH-(linker)-vL. In this disclosure, a scFv is also described as vL-(Gly-Ser-Linker)-vH.

The term “signaling domain” refers to the functional region of a protein which transmits information within the cell to regulate cellular activity via defined signaling pathways by generating second messengers or functioning as effectors by responding to such messengers.

The term “subject” is intended to include living organisms in which an immune response can be elicited (e.g., any domesticated mammal or a human).

The terms “T-cell” and “T-lymphocyte” are interchangeable and used synonymously herein. Examples include but are not limited to naïve T cells (“lymphocyte progenitors”), central memory T cells, effector memory T cells, stem memory T cells (T_{scm}), iPSC-derived T cells, synthetic T cells or combinations thereof.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Tumor,” as used herein refers to all neoplastic cell growth and proliferation, whether malignant or benign, and all pre-cancerous and cancerous cells and tissues.

The disclosure provides methods and compositions for producing CAR-T cells in vivo vs. ex vivo. The compositions include recombinant vectors (e.g., viral vectors) that contain chimeric antigen receptor (CAR) coding sequences that can be integrated into T-Cells or immune cells in vivo. The methods of the disclosure include the introduction of recombinant vectors carrying CAR coding sequences in vivo (e.g., by intravenous administration).

This disclosure provides for the intravenous delivery of vectors encoding chimeric antigen receptors (CARs) to patient lymphocytes (e.g., T cells) or other immune cells in vivo using retroviral non-replicating virus (RNV) or replication competent viruses (RRV), thus avoiding the cumbersome ex vivo transduction strategies currently being employed. T cells expressing chimeric antigen receptors (CARs) are termed CAR T cells—this encompasses both synthetic non-HLA restricted hybrid molecules, for example, an external target antigen binding site from a monoclonal antibody, and also modified or unmodified HLA restricted conventional T cell receptor subunits that recognize intracellular HLA restricted epitope presentation. RNVs containing chimeric antigen receptor coding sequences are termed RNVCAR. T-cells transfected with an RNVCAR that express the CAR are referred to as gamma CAR-T cells RNV.

The disclosure is based upon the identification of number of factors including: (a) the production of large amounts of high titer crude RNVCAR from retroviral vector producer lines, optionally in serum free and/or suspension culture; (b) purification and concentration of the vector to eliminate antigenic protein contaminants so that vector can be safely administered IV to patients as pharmaceutical preparations; (c) the observation that a small proportion of the vector gets taken up by patient T cells and this slowly decays over time; (d) the realization that specific CAR T cells that recognize a target will then amplify in vivo to achieve therapeutic benefit; (e) that because the gamma retrovirus will only effectively infect replicating cells, IV administration leads to transduction of activated T cells, because these will be a large percentage of accessible replicating cells in the blood. The same logic allows the targeted transduction of specifically stimulated/mobilized cells, such as CD34 hematopoietic stem cells mobilized with granulocyte colony stimulating factor (GCSF) (A. Publicover et al., Brit. J. Haematology, 162: 107-111, 2013). The circulating population of T cells can be further primed for vector uptake in vivo by administration of various clinically acceptable T cell stimulatory strategies including, but not limited to: vaccination with live or attenuated viral vaccines such as measles, vaccinia or some flu vaccines; cytokine stimulation (IL2 and others); administration of allergy panel tests, various super antigens in clinically acceptable human doses, monoclonal antibodies such as OKT3 (Muromonab-CD3).

RNVCAR may also be engineered to be “safety- modified” to reduce the possibility of insertional gene activation in some target cell populations (Schambach et al., Methods in Molecular Biology, Methods and Protocols, 506: 191-205, 2009) for example with a self-inactivating (SIN) configuration. Various “SIN” configurations are known in the art.

Self-inactivating” (SIN) vectors refers to replication-defective vectors, e.g., retroviral or lentiviral vectors, in which the 3′ LTR enhancer-promoter region, known as the U3 region, has been modified (e.g., by deletion or substitution) to prevent viral transcription beyond the first round of viral replication. This is because the LTR U3 region is used as a template for the 5′ LTR U3 region during viral replication and, thus, the viral transcript cannot be made without the U3 enhancer-promoter. In a further embodiment, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included in the disclosure.

An additional safety enhancement can be provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

Engineered RNVCARs may also contain “control” genes such as prodrug activating genes including, but not limited to, herpes thymidine kinase, purine nucleoside phosphorylase, cytosine deaminase or nitroreductase, or dimerizable death or apoptosis inducing proteins such as Cas9 hybrids with mutated FK506 dimerizable tails, to delete CAR T cells after therapeutic endpoints have been met or when clinically important side effects occur (such as autoantigen or off-target-related adverse events or inadvertent transduction of tumor cells potentially rendering the tumor unrecognizable by the CAR T-cells).

Engineered RNVCAR may also contain “activation-enhancement” RNA-expression modifiers such as non-coding RNAs or proteins such as single-chain antibodies, affimers, that specifically down-regulate expression of T cell check point inhibitors such as PD-1, CTLA-4 or agonists for other appropriate immune accessory molecules. In such embodiments, the RNV would transduce patient T cells so that a CAR T cell expresses the chimeric antigen receptor, a “control” gene such as cytosine deaminase, and a shRNA that degrades PD-1 transcripts and subsequently lowers PD-1 protein levels.

The disclosure allows multiple chimeric receptors against multiple tumor associated antigens and tumor neoantigens or other disease-associated antigens to be simultaneously delivered intravenously by using a mixture of RNVCARs carrying chimeric receptors against multiple antigens and epitopes.

RNVCAR delivery of chimeric antigen receptors to T cells is not an indication specific therapeutic and may be combined with personalized cancer-, or disease-profiling to select the chimeric receptors most likely to bind to antigens and neoantigens expressed by the patient regardless of indication. The ability of separate RNVCARs to intravenously delivery chimeric receptors with varying antigen binding capabilities allows temporal treatment regimens that can allow new CARs to be delivered following selective pressure and tumor or disease adaptation that deletes the original CAR epitopes. RNVCARs may be used in combination with immunotherapeutic enhancers such as checkpoint inhibitors (e.g. anti-CTLA4 and anti-PD-1), anti-tumor escape targeted drugs (e.g. IDO-1 inhibitors and anti-TGFβ), and/or chemotherapeutics with known immune-modulating effects (e.g., temozolomide, cyclophosphamide and 5-FU).

RNV delivery of chimeric receptors to patient T cells may be enhanced through the use of T-cell activating adjuvants at time of RNVCAR intravenous injection (e.g., 3-O-desacyl-4′-monophosphoryl lipid A (MPL) and interferon gamma). RNV may be delivered lymphatically and when appropriate, to the draining lymph node of solid tumor indications where tumor-associated and neoantigens are expected to be presented.

RNV may also be pseudotyped to increase transduction efficiency of T cells (e.g. GALV, amphotropic env or measles virus glycoproteins H and F) or to direct specificity of T cell sub types (e.g., memory CD4 T cells using chimeric C-HIV envelope pseudotype or CD8 T cells with MLV-10A1 envelope).

Delivery of chimeric antigen receptors directly to patient T cells using retroviral non-replicating virus (RNV), avoids the cumbersome ex vivo transduction strategies currently being employed (See Table 3).

TABLE 3
Comparison of the steps for in vivo and ex vivo transduction
of autologous cells with a retroviral vector

| step | Ex vivo | comment | step | In vivo | comment |
|---|---|---|---|---|---|
| 1. | Prepare vector for | Usually lentiviral vectors: | 1. | Prepare vector for | gamma retroviral |
| | clinical use | difficult to scale up, will | | clinical use | vectors, more scalable, |
| | | transduce non replicating | | | to prepare are quantities |
| | | cells | | | of consistent, high |
| | | | | | titer material |
| 2. | Leukapheresis | A patient's white blood | 2. | Treat patient | Inject vector by iv |
| | | cells, including T | | | administration |
| | | cells, are extracted | | | |
| | | through a specialized | | | |
| | | blood filtration process | | | |
| | | (leukapheresis). | | | |
| | | The T cells are then | | | |
| | | cryopreserved. and | | | |
| 3. | Send cells for | sent to manufacturing | | | |
| | treatment at | facility for | | | |
| | central facility | reprogramming | | | |
| 4. | Reprogram cells | T cells are genetically | | | |
| | | modified by vector | | | |
| | | prepared in step1 to | | | |
| | | recognize cancer cells | | | |
| | | and other cells | | | |
| | | expressing a specific | | | |
| | | antigen | | | |
| 5. | Expansion | Newly created CAR• T | | | |
| | | cells undergo expansion | | | |
| 6. | QC on cells | Strict quality testing | | | |
| | | occurs prior to the | | | |
| | | release and shipment of | | | |
| | | the CAR• T cells | | | |
| | | back to the patient | | | |
| 7. | Send cells back to | | | | |
| | clinical site | | | | |
| 8. | Lympho- | Extra procedure that is | | | |
| | depleting | not without risk to | | | |
| | chemotherapy for | patient | | | |
| | the patient | | | | |
| 9. | Treat patient | Infuse modified cell | | | |
| | | preparation | | | |

In one embodiment, an RNVCAR comprising a coding domain for targeting an antigen/cognate of interested (e.g., CD19) is delivered in vivo as a pharmaceutically acceptable preparation to human patient T cells. For example, a CD19-RNVCAR is injected intravenously into the blood stream for delivery to human T cells. This intravenous delivery can be done as a bolus injection or through slow infusion over minutes to hours. Injections may be repeated over several days to increase the frequency and likelihood of obtaining transduced T cells. The population of T cells can be further primed for RNVCAR vector uptake in vivo by administration of various T cell stimulatory strategies: vaccination with live viral vaccines such as measles, vaccinia or some flu vaccines; addition of adjuvant excipients currently approved in human vaccines, T cell stimulating bacterial proteins (direct and indirect), cytokine stimulation (IL2 and others), administration of allergy panel tests, various super antigens in clinically acceptable human doses, monoclonal antibodies such as OKT3 (Muromonab-CD3), and myeloid depletion using drugs such as cyclophosphamide. RNVCAR delivery to patient T cells may be enhanced through the use of T-cell activating adjuvants at time of RNVCAR intravenous injection (e.g. 3-O-desacyl-4′-monophosphoryl lipid A (MPL) and interferon gamma). These RNVCARs may be used in combination with immunotherapeutic enhancers such as checkpoint inhibitors (e.g. anti-CTLA4 and anti-PD-1), anti-tumor escape targeted drugs (e.g. IDO-1 inhibitors and anti-TGFβ), chemotherapeutics with known immune-modulating effects (e.g. temozolimide and 5-FU), or personalized neoantigen vaccines.

An RNV may also be pseudotyped to increase transduction efficiency of T cells (e.g., GALV, amphotropic env or measles virus glycoproteins H and F) or to direct specificity of T cell sub types (e.g., memory CD4 T cells using chimeric C-HIV envelope pseudotype or CD8 T cells with MLV-10A1 envelope). Delivery of an RNVCAR (e.g., a CD19-RNVCAR) using these intravenous methods produce patient T cells that target and destroy cells, microorganisms etc. that express the antigen that is specifically recognized by the binding domain of the RNVCAR (e.g., in the case of CD19-RNVCAR transduced T-cells would target and destroy CD19⁺ cells).

In another embodiment, an RNVCAR is delivered lymphatically and when appropriate, to the draining lymph node of solid tumor indications where tumor-associated and neoantigens are expected to be presented. Direct injection of lymph nodes are similarly enhanced through the use of adjuvants and combinations as mentioned above.

In yet another embodiment of RNVCAR (e.g., CD19-RNVCAR) delivery to human T cells, the method includes addition of herpes simplex virus-thymidine kinase (HSV-TK), yeast cytosine deaminase (CD) or other suicide gene or pro-drug activator systems, to the RNVCAR as a method to deplete transduced cells in the case of adverse events or at end of treatment. For example, the RNVCAR vector encoding a CD19-CAR is engineered with TK, CD or other “control” genes (e.g., Tocagen's modified yeast cytosine deaminase; see, U.S. Pat. No. 8,722,867, the disclosure of which is incorporated herein by reference). This modified RNVCAR is delivered through the same methods/processes as described above. A RNVCAR (e.g., CD19-RNVCAR) can be tracked to the tumor using radiolabeled pyrimidine (thymidine) and purine (acycloguanosine) derivatives as reporter probes for imaging of HSV-TK enzyme activity with PET. An additional advantage of including a prodrug activating enzyme such as HSV-TK or CD is that if some low percentage of tumor cells are also transduced with the CAR vector and this event renders the transduced tumor cells non-responsive to the CAR in T cells (M. Ruella et al. Nat. Med., 24:1499-1503, 2018), such tumor cells will automatically be eliminated by a course of prodrugs such valacyclovir or 5-fluorocytosine, respectively.

The disclosure provides a recombinant nucleic acid construct comprising a nucleic acid molecule encoding a CAR targeting any of the antigens identified herein. In one embodiment, the nucleic acid sequence comprises a DNA plasmid construct comprising SEQ ID NO:3 having cloned into it a CAR construct. For example, SEQ ID NO:18 to 22 provide plasmid sequences derived from SEQ ID NO:3 and containing various CAR constructs targeting CD19.

In another embodiment, the disclosure provides an RNVCAR comprising an RNA sequence packaged into a viral capsid. The RNVCAR is obtained by transfecting a nucleic acid construct containing a CAR and comprising SEQ ID NO:3 into a packaging cell line and culturing the cell line to produce the RNVCAR. The RNVCAR will comprise SEQ ID NO:12 and comprising an RNA sequence encoding a desired CAR construct (e.g., a CAR construct targeted to a desired antigen).

The disclosure also provides a vector or vectors comprising a nucleic acid sequence or sequences encoding an RNV, RNVCAR or RNV comprising a suicide/prodrug activator sequence described herein. In one embodiment, the disclosure provides one RNVCAR construct encoded by a single vector. In another embodiment, the disclosure provides more than one vector, e.g., one vector encoding a CAR and a second vector encoding a suicide/prodrug activator gene. In one embodiment, the vector or the vectors are chosen from DNA vector(s), RNA vector(s), plasmid(s), gamma-retrovirus vector(s). In one embodiment, the vector is a gutted-gamma retroviral vector comprising LTRs and packaging sequences.

DNA and/or RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001) or by causing transient perturbations in cell membranes using a microfluidic device (see, for example, patent applications WO 2013/059343 A1 and PCT/US2012/060646).

In another embodiment, a method of treating a subject, e.g., reducing or ameliorating a hyperproliferative disorder or condition (e.g., a cancer, including, but not limited to, solid tumor, a soft tissue tumor, a blood cancer, or a metastatic lesion) in a subject is provided. As used herein, the term “cancer” is meant to include all types of cancerous growths or oncogenic processes, metastatic tissues or malignantly transformed cells, tissues, or organs, irrespective of histopathologic type or stage of invasiveness. Exemplary solid tumors include malignancies, e.g., adenocarcinomas, sarcomas, and carcinomas, of the various organ systems, such as those affecting breast, liver, lung, brain, lymphoid, gastrointestinal (e.g., colon), genitourinary tract (e.g., renal, urothelial cells), prostate and pharynx. Adenocarcinomas include cancers such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the disclosure. Examples of other cancers that can be treated or prevented include pancreatic cancer, bone cancer, skin cancer, cutaneous or intraocular malignant melanoma, uterine cancer, ovarian cancer, rectal cancer, cancer of the head or neck, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The method includes administering an RNVCAR of the disclosure comprising a CAR that specifically binds to an antigen present on a cancer cell.

Exemplary cancers whose growth can be inhibited include cancers typically responsive to immunotherapy. Non-limiting examples of cancers for treatment include renal cancer (e.g. clear cell carcinoma), melanoma (e.g., metastatic malignant melanoma), breast cancer, prostate cancer (e.g. hormone refractory prostate adenocarcinoma), colon cancer and lung cancer (e.g. non-small cell lung cancer).

The following examples are provided to further illustrate, but not limit, the disclosed invention. Other methods or variations of the following examples will be readily apparent to one of skill in the art in view of the disclosure.

### EXAMPLES

Design of Murine CD19 Chimeric Antigen Receptor Constructs for Delivery by Non-Replicating Virus In vivo. A non-replicating vector (RNV) is constructed to contain a chimeric antigen receptor (CAR). In a first example, an RNVCAR is developed and targeted against murine CD19. The murine CD19 targeting is achieved using a single-chain variable fragment (scFv) constructed from the rat hybridoma clone 1D3. The scFv contains the 1D3 variable heavy chain (V_{H}) followed by a GS linker to the 1D3 variable light chain (VL). The amino acid sequence for the murine-targeted scFv are set out in Table 4. Nucleotides encoding the murine CD19 scFv can be any codons deemed most appropriate for viral vector expression and stability. The nucleotide sequence encoding the 1D3 scFv GS-VL) can be expressed from one of: the retroviral vector promoter (e.g., LTR), an internal promoter, an internal ribosome entry site (IRES) (see, e.g., WO2010036986, incorporated herein by reference), or a protein fusion strategy (e.g., using a furin peptidase site, or 2A cassette; see, e.g., U.S. Pat. Publ. No. 2018/0251786A1, which is incorporated herein by reference in its entirety) in the retroviral vector. The scFv is followed in-frame by the nucleotide sequence encoding either human CD8 hinge domain, the murine CD8 hinge domain, or the murine CD28 hinge domain as set forth in Table 5. The hinge domain is followed in-frame by the nucleotide sequence encoding either the human CD8 transmembrane domain (TMD), the murine CD8 TMD, or the murine CD28 TMD as set forth in Table 5. The transmembrane domain is followed in-frame by the nucleotide sequence encoding either the human 4-1BB intracellular signaling domain (ICD), the murine 4-1BB ICD, or the murine CD28 ICD as set forth in Table 5. The intracellular domain is followed in-frame by the nucleotide sequence encoding either the human CD3 intracellular signaling domain (ICD) or the murine CD3 ICD as set forth in Table 5. The complete murine CD19-targeted CAR construct in an RNV vector can then be delivered intravenously (IV) or intra-splenically for optimal CAR delivery, transduction and expression in T-cells.

TABLE 4

| Murine CD19 | |
|---|---|
| scFv domain | Amino acid sequence |
| V_{H} | DIQMTQSPASLSTSLGETVTIQCQASEDIY |
| | SGLAWYQQKPGKSPQLLIYGASDLQDGVPS |
| | RFSGSGSGTQYSLKITSMQTEDEGVYFCQQ |
| | GLTYPRTFGGGTKLELK |
| | (SEQ ID NO: 7) |
| GS Linker | GGGGSGGGGSGGGGS (SEQ ID NO: 8) |
| V_{L} | EVQLQQSGAELVRPGTSVKLSCKVSGDTIT |
| | FYYMHFVKQRPGQGLEWIGRIDPEDESTKY |
| | SEKFKNKATLTADTSSNTAYLKLSSLTSED |
| | TATYFCIYGGYYFDYWGQGVMVTVSS |
| | (SEQ ID NO: 9) |

TABLE 5

| | Reference | |
|---|---|---|
| Domain | sequence | Nucleotide sequence |
| Human CD8 | NM_001768.6 | ACCACGACGCCAGCGCCGC |
| hinge | | GACCACCAACACCGGCGCC |
| | | CACCATCGCGTCGCAGCCC |
| | | CTGTCCCTGCGCCCAGAGG |
| | | CGTGCCGGCCAGCGGCGGG |
| | | GGGCGCAGTGCACACGAGG |
| | | GGGCTGGACTTCGCCTGTG |
| | | AT (SEQ ID NO: 2) |
| Murine CD8 | NM_001081110.2 | tctactactaccaagccag |
| hinge | | tgctgcgaactccctcacc |
| | | tgtgcaccctaccgggaca |
| | | tctcagccccagagaccag |
| | | aagattgtcggccccgtgg |
| | | ctcagtgaa ggggaccgg |
| | | attggacttcgcctgtgat |
| | | atttac |
| | | (SEQ ID NO: 10) |
| Murine CD28 | NM_007642.4 | Attgagttcatgtaccctc |
| hinge | | cgccttacctagacaacga |
| | | gaggagcaatggaactatt |
| | | attcacataaaagagaaac |
| | | atctttgtcatactcagtc |
| | | atctcctaagctg |
| | | (SEQ ID NO: 11) |
| human CD8 | NM_001768.6 | ATCTACATCTGGGCGCCCT |
| TMD | | TGGCCGGGACTTGTGGGGT |
| | | CCTTCTCCTGTCACTGGTT |
| | | ATCACCCTTTACTGC |
| | | (SEQ ID NO: 1) |
| Murine CD8 | NM_001081110.2 | Atctgggcacccttggccg |
| TMD | | gaatctgcgtggcccttct |
| | | gctgtccttgatcatcact |
| | | ctcatc |
| | | (SEQ ID NO: 115) |
| Murine CD28 | NM_007642.4 | ttttgggcactggtcgtgg |
| TMD | | ttgctggagtcctgttttg |
| | | ttatggcttgctagtgaca |
| | | gtggctc tttgtgttat |
| | | ctggaca |
| | | (SEQ ID NO: 116) |
| Human 4-1BB | NM_001561.5 | AAACGGGGCAGAAAGAAAC |
| ICD | | TCCTGTATATATTCAAACA |
| | | ACCATTTATGAGACCAGTA |
| | | CAAACTACTCAAGAGGAAG |
| | | ATGGCTGTAGCTGCCGATT |
| | | TCCAGAAGAAGAAGAAGGA |
| | | GGATGTGAACTG |
| | | (SEQ ID NO: 5) |
| Murine 4-1BB | NM_011612.2 | AAATGGATCAGGAAAAAAT |
| ICD | | TCCCCCACATATTCAAGCA |
| | | ACCATTTAAGAAGACCACT |
| | | GGAGCAGCTCAAGAGGAAG |
| | | ATGCTTGTAGCTGCCGATG |
| | | TCCACAGGAAGAAGAAGGA |
| | | GGAGGAGGAGGCTATGAGC |
| | | TG (SEQ ID NO: 117) |
| Murine CD28 | NM_007642.4 | AATAGTAGAAGGAACAGAC |
| ICD | | TCCTTCAAAGTGACTACAT |
| | | GAACATGACTCCCCGGAGG |
| | | CCTGGGCTCACTCGAAAGC |
| | | CTTACCAGCCCTACGCCCC |
| | | TGCCAGAGACTTTGCAGCG |
| | | TACCGCCCC |
| | | (SEQ ID NO: 118) |
| Human CD3 ζ | | AGAGTGAAGTTCAGCAGGA |
| ICD | | GCGCAGACGCCCCCGCGTA |
| | | CAAGCAGGGCCAGAACCAG |
| | | CTCTATAACGAGCTCAATC |
| | | TAGGACGAAGAGAGGAGTA |
| | | CGATGTTTTGGACAAGAGA |
| | | CGTGGCCGGGACCCTGAGA |
| | | TGGGGGGAAAGCCGAGAAG |
| | | GAAGAACCCTCAGGAAGGC |
| | | CTGTACAATGAACTGCAGA |
| | | AAGATAAGATGGCGGAGGC |
| | | CTACAGTGAGATTGGGATG |
| | | AAAGGCGAGCGCCGGAGGG |
| | | GCAAGGGGCACGATGGCCT |
| | | TTACCAGGGTCTCAGTACA |
| | | GCCACCAAGGACACCTACG |
| | | ACGCCCTTCACATGCAGGC |
| | | CCTGCCCCCTCGC |
| | | (SEQ ID NO: 4) |
| Murine CD3 ζ | NM_001113391.2 | agagcaaaattcagcagga |
| ICD | | gtgcagagactgctgccaa |
| | | cctgcaggaccccaaccag |
| | | ctctacaatgagctcaatc |
| | | tagggcgaagagaggaata |
| | | tgacgtcttggagaagaag |
| | | cgggctcgggatccagaga |
| | | tgggaggcaaacagcagag |
| | | gaggaggaacccccaggaa |
| | | ggcgtatacaatgcactgc |
| | | agaaagacaagatggcaga |
| | | agcctacagtgagatcggc |
| | | acaaaaggcgagaggcgga |
| | | gaggcaaggggcacgatgg |
| | | cctttaccagggtctcagc |
| | | actgccaccaaggacacct |
| | | atgatgccctgcatatgca |
| | | gaccctggcccct |
| | | (SEQ ID NO: 119) |

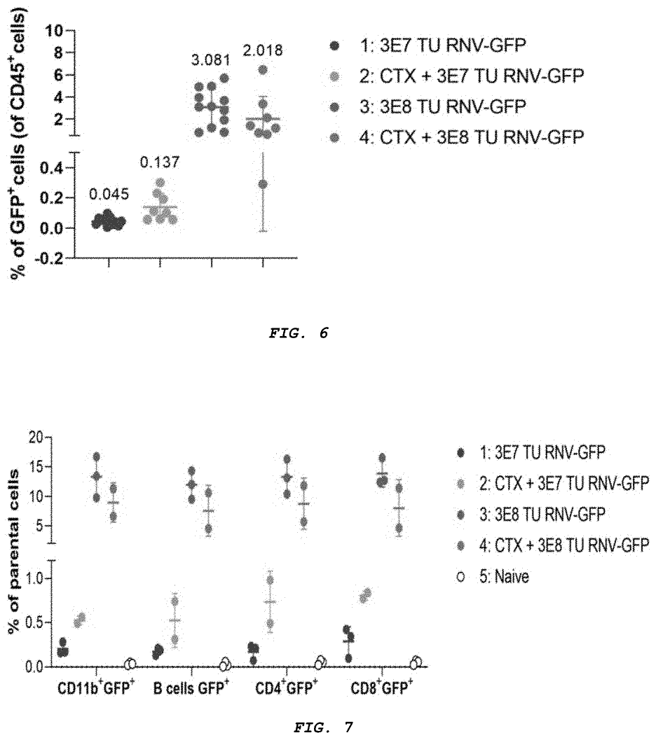

Infection of immune cells by RNV after IV administration in mice. In order to evaluate the infection potential of an RNV by IV administration, an RNV expressing green fluorescent protein (RNV-GFP) was evaluated in Balb/c mice. RNV was administered IV for 3 consecutive days at a dose of either 1E7 or 1E8 TU (titer units) per day with or without pretreatment with 100 mg/kg cyclophosphamide. Infection with RNV-GFP was determined in peripheral blood mononuclear cells (PBMC) at 7 days after the first IV dose of RNV-GFP (FIG. 6). At a total dose of 3E7 TU, 0.045% of all CD45⁺ PBMC are positive for GFP (GFP⁺). GFP⁺, RNV infected (GP⁺) CD45⁺ cells increases to 0.137% with cyclophosphamide pretreatment. At a total dose of 3E8 TU, 3.08% and 2.02% (with cyclophosphamide pretreatment) of CD45⁺ PBMC are positive for GFP Infection with RNV-GFP.

RNV-GFP infection was evaluated in a subset of PBMC populations: CD11b⁺, CD4⁺, CD8⁺, and CD19⁺ (FIG. 7). At the lower dose of RNV-GFP, total dose of 3E7 TU, less than 0.5% of each of the PBMC populations was GFP⁺. The addition of cyclophosphamide pretreatment to dose 3E7 TU generally resulted in a doubling of GFP⁺ cells in each population, about 1% GFP⁺. At the higher dose of RNV-GFP, total dose of 3E3 TU, about 10% of each of the evaluated PBMC populations was GFP⁺. The addition of cyclophosphamide pretreatment to dose 3E8 TU had no apparent impact on the percent of GFP⁺, RNV-infected cells in each evaluated PBMC population.

Treatment of a mouse model of B cell lymphoma. The A20 lymphoma line in Balb/c mice (Kueberuwa et al., J. Vis. Exp., (140), e58492, 2018, doi:10.3791/58492 2018) was used a model to evaluate the efficacy of in vivo infection of the initial mouse targeted construct, mCD19-RNVCAR (RNV-1D3CAR): mouse anti-CD19 scFv 1D3, followed by the murine CD8 transmembrane domain, followed by murine 4-1BB intracellular domain, followed by murine CD3ζ intracellular domain. Briefly, one day prior to implantation of A20 lymphoma cells, 6 to 8-week old BALB/c mice were administered 100 mg/kg cyclophosphamide intraperitoneal (IP). Cyclophosphamide pretreatment allows A20 tumor engraftment into lymphnodes without significant lymphodepletion. A20 B-cell lymphoma cells were injected IV (5E5 cells in 100 μL) on day 0. The vector, RNV-1D3CAR, was injected at a dose of 1E7 or 1E8 TU per day for five consecutive days, starting at day 5 after A20 implantation. Mice were monitored for 37 days and survival assessed.

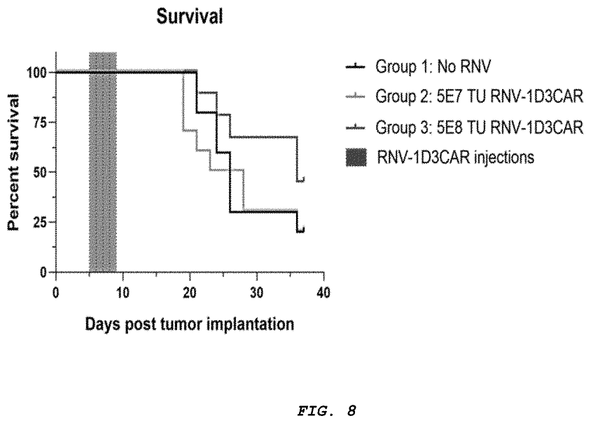

The higher dose of RNV-1D3CAR (5E8 TU) led to an improvement in survival in A20 lymphoma tumor bearing mice compared to a vehicle treated control group (no RNV) or the lower dose of RNV-1D3CAR (5E7, see FIG. 8). The improved survival of the high dose RNV-1D3CAR treated mice suggests that IV administration of mCD19-RNVCAR vector controls A20 tumor growth to some extent. For tumor control to occur after IV administration of RNV-1D3CAR, the vector must enter the circulating T cells, the mCD19-CAR must get expressed on the surface of those T cells, those T cells must then home to the tumor (primarily lymph nodes), and finally, the mCD19-CAR on the surface of the T cells must engage CD19 on the A20 tumor cells and activate killing of the A20 tumor cell.

Construction of pBA9b-hCD19CAR vectors. pBA9b (SEQ ID NO:3) provides an MLV-based retroviral non-replicating vector (RNV) containing an extended packaging region.

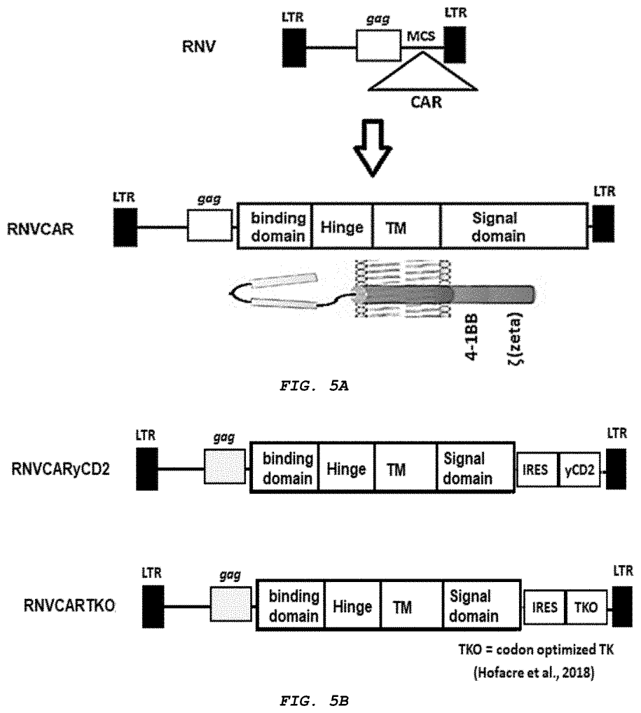

The nucleic acid sequence of various single chain variable fragment (scFv) targeting human CD19, leader sequence, hinge domain derived from human CD8, the transmembrane domain derived from human CD8, the intracellular domain derived from human 4-1BB, and the signaling domain derived from human CD3zeta are codon optimized and synthesized (Genewiz Inc.). The disclosure provides five synthesized hCD19CAR nucleic acid sequences designated: hCD19CAR1 (SEQ ID NO:13), hCD19CAR2 (SEQ ID NO:14), hCD19CAR3 (SEQ ID NO:15), hCD19CAR4 (SEQ ID NO:16), hCD19CAR5 (SEQ ID NO:17) and contain a Not I and a Sal I restriction enzyme site at 5′ and 3′, respectively, for directed cloning into the pBA9b backbone (SEQ ID NO:3) at the corresponding restriction enzyme sites (“MCS”). The resulting plasmid DNA are designated pBA9b-hCD19CAR1, pBA9b-hCD19CAR2, pBA9b-hCD19CAR3, pBA9b-hCD19CAR4, and pBA9b-hCD19CAR5 (SEQ ID NOs:18, 19, 20, 21 and 22, respectively). In all constructs, the CD19-CAR expression is mediated by the viral LTR promoter (FIG. 5A-B).

Non-clonal HAL2-hCD19CAR vector producer cell line (VPCL) produces high hCD19-RNVCAR viral titer. Non-clonal HAL2-hCD19CAR producer cells are generated to confirm viral production, hCD19CAR expression. The pBA9b-hCD19CAR vectors pseudotyped with VSV-G are first produced by transient transfection in 293GP producer cells. Subsequently, HAL2 producer cells which stably express MLV-based gag-pol and 4070A amphotropic envelop protein are transduced with multiplicity of infection (MOI) of 100. 293GP cells are derived from HEK293 cells stably producing MLV-based gag-pol. HAL2 is a human packaging cell line constructed in the same way as the VPCL HA-LB (Sheridan et al., Mol. Ther. 2000). All pBA9b-hCD19CAR vectors generated from stably transduced cells are titrated on human prostate PC-3 cells using qPCR method (5-MLV-U3-B: 5′-AGCCCACAACCCCTCACTC-3′ (SEQ ID NO:120), 3-MLV-Psi: 5′-TCTCCCGATCCCGGACGA-3′ (SEQ ID NO:121), probe: FAM-5′-CCCCAAATGAAAGACCCCCGCTGACG-3′-BHQ1 (SEQ ID NO:122)). The results show that the titer values of pBA9b-hCD19CAR vectors produced by HAL2 cells range from 5E5 to 1E7 TU/mL.

To confirm expression of hCD19CAR, 2×10⁵ hCD19CAR VPCL cells are stained with FITC-conjugated recombinant human CD19 protein (ACROBiosystems, CD9-HF2H2). Cells are analyzed on a flow cytometer (Canto, BD Biosciences) to confirm CD19 binding to hCD19CAR on the cell surface as surrogate readout for hCD19CAR expression on VPCL cell surface. In addition, hCD19CAR expression is also confirmed by anti-CD3zeta antibody (abcam, ab200591) by immunoblotting.

Clonal HAL2-hCD19CAR vector producer cell line (VPCL) produces high hCD19-RNVCAR viral titer for manufacturing. Cells from non-clonal VPCL are seeded to five 96-well plates targeting 1-cell-per-well based on cell count and limiting dilution. Wells confirmed to contain a single cell are grown to 75% confluency for screening for high titer producer clones by qRT-PCR using MLV specific primer and probe. Selected high titer producer clones are then transferred to 6-well plates followed by T75 flasks for cell expansion and further characterizations including growth characteristics, stability of retroviral components, titer, vector copy number, and transgene expression. Among the top 25 high titer producer clones selected and cultured in T75 flasks, they produce viral titer range from E6 to 2E8 TU/mL in a 5-day profiling titer assay. Subsequently, a working cell banks are generated from the highest titer clone selected and tested for a panel of safety assessment including sterility and mycoplasma, bacterial contamination, absence of RCR and other adventitious agents including bovine viruses.

In addition to the list of safety tests identified, vials from both working cell bank and master cell bank are tested to confirm cell morphology, viability, integrity of vector sequence, average vector copy number, cell doubling times and vector production by transduction titer.

hCD19-RNVCAR clinical material is produced by large scale cellular fermentation as described in Sheridan et al op.cit. and U.S. Pat. No. 10,316,333 (incorporated herein by reference), and subsequently purified (J. S. Powell et al., Blood, 102:2038-2045, 2003) by a ion exchange column then size exclusion chromatography in formulation buffer (sucrose and phosphate or Tris buffered saline), followed by filtration through 0.2 micron filters into 2 to 5m1 vials. Typically this leads to purified formulated vector preparations of 5E7 to 1E9/ml TU/ml. For GMP material this is performed at a sponsor approved GMP contract manufacturing facility.

The vector is tested for efficacy in vitro using cultured human PBMC by methods such as those described by M. C. Milone et al., Molecular Therapy, 17:1453-1464, 2009 and C. Sommer et al., Mol Ther., 27:1126-1138, 2019 and further tested in a xenograft mouse model, e.g., Milone et al., supra) using infused human PBMC followed by IV administration of the vector.

Treatment of B cell lymphoma patients. Young adult patients with CD19⁺ relapsed or refractory B-cell Acute Lymphoblastic Leukemia (ALL) are dosed iv at doses of vector of 1xE5, 1xE6, 1xE7, 1xE8, 1xE9, and 1xE10 TU/kg.

In the higher dose cohorts, the overall remission rate within 3 months is approximately 75%, with all patients who have a response to treatment found to be negative for minimal residual disease, as assessed by means of flow cytometry. The rates of event-free survival and overall survival are mostly over 60% and 90% respectively, at 6 months and mostly over 35% and 60% at 12 months. The exact numbers depend on the status of the patients initially, but are comparable to young adults in the ex vivo trial, described by Maude et al., N. Eng. J. Med., 378:439-449, 2018. The overall criteria for utility is a 3 month remission rate of >20%.

A number of embodiments of the disclosure have been described. Nevertheless, it will be understood that various modifications may be made without departing from the spirit and scope of the disclosure. Accordingly, other embodiments are within the scope of the following claims.

1. A pharmaceutical composition for administration to a subject, the pharmaceutical composition comprising:
a non-replicating viral vector containing a heterologous polynucleotide encoding a chimeric antigen receptor (CAR) or CAR-like product having an immune activating effect
wherein the non-replicating viral vector transduces or integrates into blood cells of the subject in vivo.

2. The pharmaceutical composition of claim 1, wherein the subject is a mammal.

3. The pharmaceutical composition of claim 2, wherein the mammal is a human.

4. The pharmaceutical composition of claim 1, wherein the composition is administered intraperitoneally.

5. The pharmaceutical composition of claim 1, wherein the composition is administered intravascularly.

6. The pharmaceutical composition of claim 1, wherein the non-replicating viral vector is a non-replicating retroviral vector.

7. The pharmaceutical composition of claim 6, wherein the non-replicating retroviral vector is selected from the group consisting of a gammaretroviral vector, a betaretroviral vector, an alpharetroviral vector, a lentiviral vector and a foamy viral vector.

8-9. (canceled)

10. The pharmaceutical composition of claim 7, wherein non-replicating retroviral vector is engineered from MLV.

11. (canceled)

12. The pharmaceutical composition of claim 1, wherein the non-replicating viral vector (RNV) comprises:
an envelope comprising envelope proteins and lipid membrane;
a nucleic acid contained within the envelope, the nucleic acid comprising a sequence from 5′ to 3′ comprising:
SEQ ID NO:12 from nucleotide 1 to 1050 or a sequence that is at least 85% identical thereto;
an RNA sequence encoding the chimeric antigen receptor product having the immune activating effect; and
SEQ ID NO:12 from nucleotide 1101 to 1662 or a sequence that is at least 85% identical thereto,
wherein the RNV can infect dividing cells and wherein the RNA sequence encoding the chimeric antigen receptor product having immune activating effect is integrated into the dividing cell.

13. The pharmaceutical composition of claim 1, wherein the immune activating effect is an immune mediated activity.

14. The pharmaceutical composition of claim 1, wherein the blood cells are T-cells or T-cell precursors.

15. (canceled)

16. The pharmaceutical composition of claim 1, wherein the CAR or CAR-like product comprises an antigen binding domain, an optional spacer, a transmembrane domain and an optional intracellular domain.

17. The pharmaceutical composition of claim 16, wherein the antigen binding domain is a non-immunoglobulin binding domain.

18. The pharmaceutical composition of claim 17, wherein the non-immunoglobulin binding domain comprises a scaffold protein.

19. The pharmaceutical composition of claim 16, wherein the transmembrane domain is a CD28 transmembrane domain.

20. The pharmaceutical composition of claim 16, wherein the transmembrane domain is a CD8 transmembrane domain.

21. The pharmaceutical composition of claim 20, wherein the CD8 transmembrane domain is encoded by a sequence that is 98% to 100% identical to SEQ ID NO:1.

22. The pharmaceutical composition of claim 16, wherein the antigen binding domains comprises an scFv or antibody fragment.

23. The pharmaceutical composition of claim 16, wherein the optional spacer is encoded by a sequence that is at least 98% identical to SEQ ID NO:2.

24. The pharmaceutical composition of claim 16, wherein the optional intracellular domain comprises a CD3ζ domain.

25. The pharmaceutical composition of claim 16, wherein the optional intracellular domain comprises a 41 BB or CD28 intracellular domain.

26. The pharmaceutical composition of claim 25, wherein the 41BB domain is encoded by a sequence that is 98% to 100% identical to SEQ ID NO:5.

27. The pharmaceutical composition of claim 24, wherein the CD3ζ domain is encoded by a sequence that is at least 98% identical to SEQ ID NO:4.

28. The pharmaceutical composition of claim 16, wherein the antigen-binding domains specifically binds to an antigen selected from the group consisting of:
CD5, CD19; CD123, CD22, CD30, CD171; CS1 (also referred to as CD2 subset 1, CRACC, SLAMF7, CD319, and 19A24); C-type lectin-like molecule-1 (CLL-1 or CLECL1), CD33; epidermal growth factor receptor variant III (EGFRviii);
ganglioside G2 (GD2); ganglioside GD3 (aNeu5Ac(2-8)aNeu5Ac(2-3)bDGalp(I-4)bDGlcp(1-1)Cer), TNF receptor family member B cell maturation (BCMA); Tn antigen ((Tn Ag) or (GaINAcα-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra), prostate stem cell antigen (PSCA); Protease Serine 21 (Testisin or PRSS21), vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20, Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(I-4)bDGlcp(I-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ES0-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member IA (XAGEI); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator oflmprinted Sites), Squamous Cell Carcinoma Antigen Recognized By T Cells 3 (SART3); Paired box protein Pax-5 (PAXS); proacrosin binding protein sp32 (OY-TESI); l ymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1), renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRD, Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLI), MPL, Biotin, c-MYC epitope Tag, CD34, LAMP1 TROP2, GFRalpha4, CDH17, CDH6, NYBR1, CDH19, CD200R, Slea (CA19.9, Sialyl Lewis Antigen); Fucosyl-GM1, PTK7, gpNMB, CDH1-CD324, DLL3, CD276/B7H3, IL11Ra, IL13Ra2, CD179b-IGLI1, TCRgamma-delta, NKG2D, CD32 (FCGR2A), Tn ag, Tim1-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLV1-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRCSD, Claudin18.2 (CLD18A2 or CLDN18A.2)), P-glycoprotein, STEAP1, Liv1, Nectin-4, Cripto, gpA33, BST1/CD157, low conductance chloride channel, and the antigen recognized by TNT antibody.

29. The pharmaceutical composition of claim 1, wherein the non-replicating viral vector comprises a self-inactivating nucleic acid sequence (SIN).

30. The pharmaceutical composition of claim 1, wherein the viral vector is produced by expression of a sequence selected from the group consisting of SEQ ID NO:18, 19, 20, 21, and 22.

31. A non-replicating viral vector comprising:
an envelope comprising envelope proteins and lipid membrane;
a nucleic acid contained within the envelope, the nucleic acid comprising a sequence from 5′ to 3′ comprising:
SEQ ID NO:12 from nucleotide 1 to 1050 or a sequence that is at least 85% identical thereto;
an RNA sequence encoding a chimeric antigen receptor (CAR); and
SEQ ID NO:12 from nucleotide 1101 to 1662 or a sequence that is at least 85% identical thereto,
wherein the RNV can infect dividing cells and wherein the RNA sequence encoding the chimeric antigen receptor is integrated into the dividing cell.

32. The RNVCAR of claim 31, wherein the CAR comprises an antigen binding domain, an optional spacer, a transmembrane domain and an optional intracellular domain.

33. The RNVCAR of claim 32, wherein the transmembrane domain is a CD28 transmembrane domain.

34. The RNVCAR of claim 32, wherein the transmembrane domain is a CD8 transmembrane domain.

35. The RNVCAR of claim 34, wherein the CD8 transmembrane domain is encoded by a sequence that is 98% to 100% identical to SEQ ID NO:1.

36. The RNVCAR of claim 32, wherein the antigen binding domains comprises an scFv or antibody fragment or non-immunoglobulin binding domain.

37. The RNVCAR of claim 32, wherein the optional spacer is encoded by a sequence that is at least 98% identical to SEQ ID NO:2.

38. The RNVCAR of claim 32, wherein the intracellular domain comprises a CD3ζ domain.

39. The RNVCAR of claim 32, wherein the intracellular domain comprises a 41BB or CD28 intracellular domain.

40. The RNVCAR of claim 39, wherein the 41BB domain is encoded by a sequence that is 85% to 100% identical to SEQ ID NO:5.

41. The RNVCAR of claim 38, wherein the CD3 domain is encoded by a sequence that is at least 98% identical to SEQ ID NO:4.

42. The RNVCAR of claim 31, wherein the RNA sequence encoding the chimeric antigen receptor comprises any one of SEQ ID NOs: 13 to 17 wherein T is U.

43. The RNVCAR or claim 32, wherein the antigen-binding domains specifically binds to an antigen selected from the group consisting of:
CD5, D19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(I-4) bDGlcp(I-I)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra), prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(I-4)bDGlcp(I-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ES0-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member IA (XAGEI); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI), Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYON); Ras Homolog Family Member C (RhoC); Tyrosinase-related protein 2 (TRP-2); Cytochrome P450 IB 1 (CYPIB 1), CCCTC-Binding Factor (Zinc Finger Protein)-Like (BORIS or Brother of the Regulator ofImprinted Sites), Squamous Cell Carcinoma Antigen Recognized By T Cells 3 (SART3); Paired box protein Pax-5 (PAX5); proacrosin binding protein sp32 (OY-TESI); lymphocyte-specific protein tyrosine kinase (LCK); A kinase anchor protein 4 (AKAP-4); synovial sarcoma, X breakpoint 2 (SSX2)l Receptor for Advanced Glycation Endproducts (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI), Fc fragment of IgA receptor (FCAR or CD89); Leukocyte immunoglobulin-like receptor subfamily A member 2 (LILRA2);, CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLI), MPL, Biotin, c-MYC epitope Tag, CD34, LAMP1 TROP2, GFRalpha4, CDH17, CDH6, NYBR1, CDH19, CD200R, Slea (CA19.9, Sialyl Lewis Antigen); Fucosyl-GM1, PTK7, gpNMB, CDH1-CD324, DLL3, CD276/B7H3, IL11Ra, IL13Ra2, CD179b-IGLI1, TCRgamma-delta, NKG2D, CD32 (FCGR2A), Tn ag, Tim1-/HVOR1, CSF2RA (GM-CSFR-alpha), TGFbetaR2, Lews Ag, TOR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLV1-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudin18.2 (CLD18A2 or CLDN18A.2)), P-glycoprotein, STEAP1, Liv1, Nectin-4, Cripto, gpA33, BST1/CD157, low conductance chloride channel, and the antigen recognized by TNT antibody.

44. The RNVCAR of claim 31, further comprising an IRES or 2A peptide cassette operably linked to a heterologous polynucleotide.

45. The RNVCAR of claim 44, wherein the heterologous polynucleotide encodes a prodrug activator enzyme.

46. The RNVCAR of claim 45, wherein the prodrug activator enzyme is cytosine deaminase or thymidine kinase.

47. The RNVCAR of claim 31, produced by transfecting a packaging cell line with a plasmid comprising SEQ ID NO:3 containing a chimeric antigen receptor coding sequence, culturing the packaging cell to produce the RNVCAR and isolating the RNVCAR.

48. The RNVCAR of claim 47, wherein the plasmid comprises a sequence selected from the group consisting of SEQ ID NO:18 to 22.

49. A method of providing adoptive cell therapy to a subject comprising:
administering a pharmaceutical composition of claim 1 or an RNVCAR of claim 31 to the subject.

50. The method of claim 49, wherein the subject is treated with an agent to activate T-cells prior to or simultaneously with the administration of the RNVCAR or pharmaceutical composition.
